(19) United States
(12) Patent Application Publication
(10) Pub. No.: US 2024/0342282 A1
(43) Pub. Date: Oct. 17, 2024

(54) ANTI-CSF1R CAR EXPRESSING LYMPHOCYTES FOR TARGETED TUMOR THERAPY

(71) Applicant: Ludwig-Maximilians-Universität München, München (DE)

(72) Inventors: Adrian Gottschlich, München (DE); Stefanie Lesch, München (DE); Stefan Endres, München (DE); Sebastian Kobold, München (DE)

(21) Appl. No.: 18/683,237

(22) PCT Filed: Aug. 12, 2022
(86) PCT No.: PCT/EP2022/072693

§ 371 Date: Feb. 12, 2024

(30) Foreign Application Priority Data
Aug. 13, 2021 (EP) 21191376.9

(51) Int. Cl. A61K 39/00; A61P 35/00; C07K 14/725; C07K 16/28; C12N 5/0783

(52) U.S. Cl. CPC A61K 39/464418; A61K 39/4611; A61K 39/4631; A61P 35/00; C07K 14/7051; C07K 16/28; C12N 5/0636; C07K 2317/33; C07K 2317/622; C07K 2317/732; C12N 2501/2302; C12N 2501/2315

(57) ABSTRACT

The present invention relates to the recognition of CSF1R as a marker of hematological cancer and thus relates to CSF1R targeting agents for the treatment of such cancers, in particular, AML. The invention also relates to a lymphocyte recombinantly expressing a chimeric antigen T cell receptor (CAR) specific for CSF1R, in particular, for use in the treatment of cancer characterized by the expression of colony stimulating factor 1 receptor (CSF1R). The present invention further relates to a CAR comprising an extracellular domain that specifically binds CSF1R, a transmembrane domain, and an intracellular T cell activating domain; as well as polynucleotides, vectors and host cells used in the production of the CAR. Further, methods for the production of such lymphocytes and a pharmaceutical composition comprising such lymphocytes are provided. The cells of the invention are preferably human lymphocytes and more preferably primary human lymphocytes such as CD3+ T cells, CD8+ T cells, CD4+ T cells, γδ T cells, invariant T cells or NK T cells.

### 1. BACKGROUND

The present invention relates to the recognition of CSF1R as a marker of hematological cancer and thus relates to CSF1R targeting agents for the treatment of such cancers, in particular, AML. The invention also relates to a lymphocyte recombinantly expressing a chimeric antigen T cell receptor (CAR) specific for CSF1R, in particular, for use in the treatment of cancer characterized by the expression of colony stimulating factor 1 receptor (CSF1R). The present invention further relates to a CAR comprising an extracellular domain that specifically binds CSF1R, a transmembrane domain, and an intracellular T cell activating domain; as well as polynucleotides, vectors and host cells used in the production of the CAR. Further, methods for the production of such lymphocytes and a pharmaceutical composition comprising such lymphocytes are provided. The cells of the invention are preferably human lymphocytes and more preferably primary human lymphocytes such as CD3+ T cells, CD8+ T cells, CD4+ T cells, γδ T cells, invariant T cells or NK T cells.

T cells have been established as major target structures and effectors in oncology (Kobold et al., 2015). One example for an approach using T cells as indirect therapeutic target are so called checkpoint inhibitors that increase T cell activity. These checkpoint inhibitors have been established as an important element in the treatment of an increasing number of malignant diseases at an advanced stage, such as the melanoma, non-small-cell lung carcinoma or the Hodgkin lymphoma (Kobold et al., Front Oncol. (2018); 25; 8:285. eCollection). The basis for their approval is a significantly prolonged overall survival of the treated patients. It has been demonstrated that T cells can be activated and targeted against a plethora of malignant diseases, however, the efficiency of these molecules (i.e. the checkpoint inhibitors) in treatment of acute leukemia remains elusive (Boekstegers et al., Bone Marrow Transplant. (2017); 52(8):1221-1224.

The remarkable achievements using checkpoint inhibitors that increase T cell activity suggest that T cells may be used successfully as direct therapeutic targets. One approach is the adoptive cell therapy (ACT) using autologous tumor-infiltrating lymphocytes (TIL), wherein patients with metastasizing tumor-diseases first undergo surgery and samples of disease tissue are isolated, TILs are isolated from the tissue sample and subsequently expanded/stimulated in vitro.

Another approach is the retroviral transduction of autologous or donor T cells obtained from peripheral donor blood. These cells are either transduced with a tumor-associated antigen-specific T cell receptor (TCR) or with a chimeric antigen receptor (CAR), which recognizes specific structures on the surface of tumor cells (Grupp et al., N Engl J Med. (2013); 18; 368(16):1509-1518.; Morgan et al., J Immunother. (2013); 36(2):133-51, 2013). Such “CAR T cells” are considered to couple the specificity of an antibody with the destructive force of T cell effector functions (Benmebarek et al., Int J Mol Sci. (2019); 14; 20(6)), thereby constituting a powerful approach to ACT. The first therapies have recently been approved in the US and Europe, e.g. for the treatment of B-cell-associated refractory acute lymphatic leukemia (Maude et al., N Engl J Med. (2018); 1; 378(5):439-448). These therapies comprise so called chimeric antigen receptors (CAR)-modified T-cells that detect the B-cell-associated antigen CD19. High rates of remission and a significantly prolonged overall survival of the treated patients have been observed, proving the potency of such cell therapies.

Autologous T cell transfer is currently approved for only two leukemic indications, both using anti-CD19-CAR-Tcells. However, autologous CAR T cell therapy is currently being tested for additional indications. Regardless of these promising achievements, the prognosis of patients suffering from refractory or relapsing acute myeloid leukemia remains consistently poor (Megias-Vericat et al., Ann Hematol. (2018); 97(7):1115-115). One possible reason is speculated to be that that the immunotherapies lack relevant target specificity and are associated with toxicity via on-target-off-AML detection (Gattinoni et al., Nat Rev Immunol. (2006); 6(5):383-93).

One potential target used for detection of refractory or relapsing acute myeloid leukemia is CD33, a surface marker broadly expressed on myeloid cells, which is currently being investigated as target structure for the development of various therapeutic antibodies (Schurch et al., Front Oncol. (2018); 18; 8:152). As applied to ACT, initial studies using T cells engineered to express an anti-CD33 chimeric antigen receptor (anti-CD33-CAR-T cells) have shown promising anti-tumor potency. However, it is also associated with severe side-effects, similar to those of anti-CD19-CAR-T cells (Wang et al., Mol Ther. (2015); 23(1):184-91). Again, the side-effects are believed to be caused by an insufficient target specificity.

Accordingly, what is necessary is the identification of a more promising target molecule. An ideal target structure for AML should be expressed on AML cells as broadly and homogenously as possible, but not on cells of the healthy hematopoiesis (or at least only on infrequently occurring subtypes). Currently, no strictly AML- or cancer-specific surface antigens have yet been identified (He et al., Blood. (2020); 5; 135(10):713-723.). This is believed to be because such target structures are also highly likely to be expressed on cells of the healthy hematopoiesis or related cell types (which also explains the majority of the expected and observed toxicity associated with the targeting of such antigens by the various therapies tested thus far). In contrast, target structures that are not significantly expressed on healthy cells have the disadvantage that they are not typically uniformly expressed on AML-blasts, or are only expressed in specific AML subtypes limiting general applicability. Thus, the expected benefit therapies targeting the more restricted markers is reduced and a long-lasting therapeutic effect is prevented.

The present inventors have identified the molecule colony stimulating factor 1 receptor (CSF1R) as a broadly expressed AML target-structure. CSF1R was known to be expressed in vivo on distinct myeloid subpopulation, such as the M2-macrophages (Ries et al., Cancer Cell. (2014); 16; 25(6):846-59). In the context of tumor-diseases, CSF1R plays an important role mainly in immunosuppression (Ries et al., Cancer Cell. (2014); 16; 25(6):846-59). CSF1R-specific small-molecule-inhibitors as well as monoclonal antibodies have been developed in this context (Edwards et al., Blood. (2019); 7; 133(6):588-599; Ries et al., Cancer Cell. (2014); 16; 25(6):846-59) and are already under examination in clinical studies for the treatment of AML (NCT03557970). First clinical studies indicate that the depletion of CSF1R-expressing cell populations is not expected to induce special side-effects. Furthermore, a recent study used CSF1R as an exemplary target structure to demonstrate the potential for CAR T cell therapy. The exemplified CAR molecule was a third-generation CAR T cell that specifically recognized human CSF1R (Zhang et al., Immunotherapy (2018), 10(11), 935-949). The study reports in vitro CAR T cell-induced cytotoxicity against two cancer cell lines expressing CSF1R. However, the target cell lines were genetically engineered to recombinantly express CSF1R. Therefore, the teaching related to the potential of third generation CAR constructs generally and provided no demonstration of real-world clinical value.

### 2. SUMMARY

In the context of AML, an amplification of CSF1R signaling and the therapeutic potential of its inhibition has only been described for rare subtypes (Edwards et al., Blood. (2019); 7; 133(6):588-599). However, a broad expression of CSF1R and a broad application of this signaling pathway has been denied (Aikawa et al., Nature Medicine (2010); (16):580-585 Edwards et al., Blood. (2019); 7; 133(6):588-599.). Accordingly, CSF1R is not recognized as a suitable target structure for AML due to its putative low expression. The present inventors have surprisingly and unexpectedly found that, contrary to the reports in the art, CSF1R provides a surprisingly effective target for T-cell-based therapies. As evident from the appended Examples, CSF1R is shown to be expressed on the majority of AML-subtypes while the expression on healthy cells is limited to distinct myeloid subpopulation, such as M2-macrophages. Accordingly, provided are improvements based on the identification of CSF1R as a ubiquitous target structure in AML with limited expression on normal cells, including the provision of efficient anti-CSF1R-CAR constructs, and anti-CSF1R-CAR lymphocytes with demonstrated in vitro and in vivo efficacy.

The present invention provides a lymphocyte recombinantly expressing a chimeric antigen T cell receptor (CAR) for use in the treatment of cancer characterized by the expression of colony stimulating factor 1 receptor (CSF1R; exemplary UniProt accession no: P07333 and Gene Bank gene ID: 1436). The CAR construct comprises an extracellular domain that specifically binds CSF1R, a transmembrane domain, and an intracellular T cell activating domain. As used herein, the term recombinantly expresses the CAR is used as commonly understood in the art, indicating that the cell and/or its progenitor cell/cell line has been genetically engineered to express the CAR construct. Accordingly, the CAR T cell disclosed herein comprises nucleic acid sequences not endogenously found in T cells, e.g. comprising promoter sequences operably linked to cDNA sequences encoding one or more portions of the CAR as disclosed herein such as (i) an extracellular domain that specifically binds CSF1R, (ii) a transmembrane domain and (iii) an intracellular T cell activating domain.

The extracellular domain of the CAR as described herein comprises an antigen binding region specific for CSF1R. The antigen binding region as described herein may be any moiety providing specificity for the antigen CSF1R or any epitope thereof, but is preferably an antigen-binding region derived from an antibody including but not limited to antigen binding fragments derived from the Fv domain. Exemplary antigen-binding regions derived from an antibody Fv domain include (but are not limited to) paired heavy and light chain variable domains, such as Fab, Fab′, F(ab′)₂, and Fv fragments as well as recombinant constructs such as single-chain Fv domains, known in the art as scFvs. It is preferred that the antibody-derived antigen-binding region is an scFv. Any scFv known in the art or described herein specific for CSF1R (whether human, humanized or derived from an antibody of a non-human animal, e.g. mouse) can be used in the construction of the CAR or lymphocyte and/or in the treatment of cancer as disclosed herein. It is most preferred that the scFv is a human or humanized scFv. CSF1R-specific humanized scFvs are known in the art and include that derived from clone 2F11-e7 as disclosed in EP-B1 2 510 010 (SEQ ID NO:1) and that derived from 1.2.1 SM as disclosed in WO 2009/036303 (SEQ ID NO:2), which may be encoded, for example, by SEQ ID NO:3 and SEQ ID NO:4, respectively.

Antigen-binding regions derived from an antibody as used herein also include antibody antigen binding fragments comprising a single, unpaired heavy or light chain variable domain as known in the art that retains the ability to specifically and selectively bind antigen (CSF1R), including but not limited to single domain antibodies (also referenced in the art as sdAbs, dAbs, and/or nanobodies) and VHH domains based on the heavy chains of camelids.

As noted above, is most preferred that the extracellular domain of the CAR as disclosed herein comprises an antigen-binding region that comprises or consists of human or humanized scFv sequences, which, in non-limiting embodiments, may be an antigen-binding region comprising or consisting of the amino acid sequence of SEQ ID NO:1 or SEQ ID NO:2. The antigen-binding region may alternatively comprise or consist of a SEQ ID NO:1 or SEQ ID NO:2 variant amino acid sequence, which variant amino acid sequence is defined herein as having an amino acid sequence that is at least 85%, 86%, 87%, 88%, 89%, 90%, 91%, 92%, 93%, 94%, 95%, 96%, 97%, 98% or 99% identical to SEQ ID NO:1 or SEQ ID NO:2, respectively, and is further characterized by specific binding to CSF1R. It is preferred that the SEQ ID NO:1 variant amino acid sequence or the SEQ ID NO:2 variant amino acid sequence has at least 85% sequence identity to SEQ ID NO:1 or SEQ ID NO:2, respectively (and, again, exhibits CSF1R specific binding activity). The antigen binding region may alternatively comprise or consist of a fragment of SEQ ID NO:1, SEQ ID NO:2, a SEQ ID NO:1 variant amino acid sequence, or a SEQ ID NO:2 variant amino acid sequence, which fragment is characterized by specific binding to CSF1R. Specific binding activity to CSF1R is preferably tested via recombinant protein binding assays, whether cell or polypeptide based, as known in the art. In an alternative nonlimiting example, specific binding activity to CSF1R is measured in the context of the CAR as expressed in a T cell by assessing T cell activation in response to antigen (i.e. T cell activation on binding to CSF1R). In a non-limiting example, increasing concentrations of Fc or HIS-tagged recombinant CSF1R protein are coated on a plate and incubated overnight at 4° C. Following blocking and washing, CAR-transduced T cells are added to the plate and T cell activation is measured with flow cytometry as known in the art or described herein. Increased T cell activation correlating with increasing concentration of the CSF1R protein indicates specific binding activity of the CAR for CSF1R.

The extracellular domain may further comprise additional regions, e.g. a peptide spacer connecting the antigen binding region to the transmembrane domain of the CAR. The optional peptide spacer within the extracellular domain of the CAR of the invention comprises a flexible amino acid sequence connecting the antigen binding region to the transmembrane domain. The flexible spacer allows the antigen-binding region to orient in different directions to facilitate ligand recognition and binding. It is preferred that the spacer region does not promote secondary structures and/or does not adopt three-dimensional structures. It is further preferred that the spacer is biologically neutral (other than optionally having a tag function as described herein). That is, it is preferred that the spacer does not have biological activity, e.g. interact with one or more receptors or ligands endogenously expressed by the cell expressing the CAR and/or the subject to which the cell expressing the CAR is to be administered. Thus, it is preferred that the spacer does not consist or comprise a ligand or receptor (or portion thereof) that interacts with a counterpart receptor, endogenously expressed by either (1) the cell expressing the CAR of the invention or (2) the subject to be administered the T cell of the invention. It is most preferred that the optional spacer of the extracellular domain does not comprise an antibody Fc domain or domain thereof, e.g. does not comprise a CH1 domain, a CH2 domain, a CH3 domain, or a biologically active fragment thereof.

The optional spacer as described herein may comprise a hinge region as is known in the art or described herein. Any extracellular part of a protein comprising an extracellular domain, e.g. as provided among others by the CD nomenclature, may be used as a hinge domain in the extracellular domain of the CAR of the invention. Exemplary spacers include, without limitation, a CD8 hinge domain, a CD28 hinge domain, a TLR5 hinge domain, and a CSF1R linker domain. Any hinge domain known in the art or described herein can be used in the disclosed CARs and in the practice of the disclosed methods, including hinge domains from non-human or human proteins. An exemplary CD8 murine hinge domain amino acid sequence is SEQ ID NO:5 (which may be, for example, encoded by SEQ ID NO:6). An exemplary CD8 human hinge domain amino acid sequence is SEQ ID NO:7 (which may be, for example, encoded by SEQ ID NO:8). An exemplary CD28 human hinge domain amino acid sequence is SEQ ID NO:9 (which may be, for example, encoded by SEQ ID NO:10). In embodiments of the CAR of the invention comprising a spacer, it is preferred that the spacer comprises or consists of a human hinge domain. It is most preferred that in embodiments of the CAR of the invention comprising a spacer that the spacer comprises or consists of a human CD8 hinge domain. Thus, a non-limiting example of this most preferred embodiment is a CAR comprising a spacer comprising or consisting of SEQ ID NO:7.

Where the extracellular domain of the CAR of the invention (e.g. expressed by the lymphocyte of the invention) comprises a spacer, the spacer may further comprise a detectable tag (e.g. peptide sequence) allowing detection and/or purification of the extracellular domain, the (expressed) CAR and/or cell expressing the CAR. Suitable tags allowing detection and/or purification are known in the art and include but are not limited to protein tags (e.g. HIS-tag, HA-tag, c-myc-tag, FLAG-tag), bi- or polycistronic vectors containing truncated proteins (examples include but are not limited to CD19, CD20, CD34, epidermal growth factor receptor (EGFR) or intracellular or transmembrane-located fluorescent proteins (e.g. enhanced green fluorescent protein (eGFP)) (Hu and Huang, Front. Immunol. (2020); 11: 1770). A preferred non-limiting example of a detectable tag is a c-myc tag. As known in the art, the c-myc tag is a peptide derived from the c-myc gene product allowing the detection and/or purification of the polypeptide comprising it and/or the cell expressing the polypeptide comprising the c-myc tag. In a non-limiting embodiment, the c-myc tag comprises or consists of the amino acid sequence SEQ ID NO:11 (which may be, for example, encoded by the nucleic acid sequence SEQ ID NO:12).

Accordingly, the extracellular domain of the CAR of the invention (e.g., expressed by the lymphocyte for use of the invention) preferably comprises (i) a human or humanized scFv antigen binding region specific for CSF1R and (ii) an optional spacer comprising a human hinge region with an optional detection/purification tag. It is preferred that the optional spacer does not comprise an antibody Fc region or portion thereof, and/or does not have binding activity for one or more Fc receptors, e.g. FcγR and/or FcRn. It is most preferred that the extracellular domain of the CAR of the invention (e.g., expressed by the lymphocyte for use of the invention) comprises (i) a human or humanized scFv antigen binding region specific for CSF1R and (ii) a spacer comprising a human hinge region with a detection/purification tag.

A non-limiting example of the above-described most preferred embodiment of the CAR of the invention (or of the CAR recombinantly expressed by the lymphocyte of the invention and for the use of the invention) comprises an extracellular domain comprising or consisting of
(A) an antigen binding region that comprises or consists of
   (i) the amino acid sequence of a human or humanized scFv specific for CSF1R;
   (ii) an amino acid sequence that is e.g. at least 85% identical to the amino acid sequence of (i) characterized by specifically binding CSF1R; or
   (iii) a fragment of the amino acid sequence of (i) or (ii) characterized by specifically binding CSF1R; and
(B) a spacer comprising a human hinge region and a c-myc tag.

As a specific example of such a most preferred embodiment, the CAR of the invention (or of the CAR recombinantly expressed by the lymphocyte of the invention and for the use of the invention) comprises an extracellular domain comprising or consisting of
(A) an antigen binding region that comprises or consists of
   (i) the amino acid sequence of a SEQ ID NO:1 or SEQ ID NO:2;
   (ii) an amino acid sequence that is e.g. at least 85% identical to the amino acid sequence of SEQ ID NO:1 or SEQ ID NO:2 characterized by specifically binding CSF1R; or
   (iii) a fragment of the amino acid sequence of (i) or (ii) characterized by specifically binding CSF1R; and
(B) a spacer comprising or consisting of SEQ ID NO:13 or SEQ ID NO:14.

The extracellular domain of this most preferred embodiment further (i) does not comprise an antibody Fc region or portion thereof, and/or (ii) does not have binding activity for one or more Fc receptors.

A further specific example of such a most preferred embodiment of the CAR of the invention explained above (or of the CAR recombinantly expressed by the lymphocyte of the invention and for the use of the invention) comprises an extracellular domain comprising or consisting of
(A) the amino acid sequence of SEQ ID NO:15, SEQ ID NO:16, SEQ ID NO:17, or SEQ ID NO:18;
(B) an amino acid sequence that is e.g. at least 85% identical to the amino acid sequence of SEQ ID NO:15, SEQ ID NO:16, SEQ ID NO:17, or SEQ ID NO:18 characterized by specifically binding CSF1R and having a c-myc tag; or
(C) a fragment of the amino acid sequence of (i) or (ii) characterized by specifically binding CSF1R and having a c-myc tag.

The extracellular domain of this most preferred embodiment further (i) does not comprise an antibody Fc region or portion thereof, and/or (ii) does not have binding activity for one or more Fc receptors.

Therefore, the invention also provides a lymphocyte recombinantly expressing a CAR comprising (i) an scFv antigen binding region specific for CSF1R and (ii) an optional spacer comprising a hinge region with an optional detection/purification tag. It is preferred that the scFv antigen binding region is a human or humanized scFv. As a specific example of such a preferred embodiment, the antigen binding region of the CAR recombinantly expressed by the lymphocyte of the invention comprises or consists of SEQ ID NO:1 or SEQ ID NO:2, an amino acid sequence variant of SEQ ID NO:1 or SEQ ID NO:2, which variant is an amino acid sequence at least 85% identical to SEQ ID NO:1 or SEQ ID NO:2, or a fragment of SEQ ID NO:1 or SEQ ID NO:2 or a fragment of the sequence variant of SEQ ID NO:1 or SEQ ID NO:2, which variant or fragment is characterized by specifically binding CSF1R. It is most preferred that the extracellular domain of the CAR expressed by the lymphocyte of the invention comprises both an antigen-binding region that is a human or humanized scFv (or variant amino acid sequence or fragment as described above) and a spacer comprising a human hinge region and a detection/purification tag. In one example of such a most preferred embodiment of a lymphocyte of the invention expresses a CAR comprising an extracellular domain comprising or consisting of
(A) an antigen binding region that comprises or consists of
   (i) the amino acid sequence of SEQ ID NO:1 or SEQ ID NO:2;
   (ii) an amino acid sequence that is e.g. at least 85% identical to SEQ ID NO:1 or SEQ ID NO:2 characterized by specifically binding CSF1R; or
   (iii) a fragment of the amino acid sequence of (i) or (ii) characterized by specifically binding CSF1R; and
(B) a spacer comprising or consisting of SEQ ID NO:13 or SEQ ID NO:14.

The extracellular domain of this example further (i) does not comprise an antibody Fc region or portion thereof, and/or (ii) does not have binding activity for one or more Fc receptors.

A further specific example of such a most preferred embodiment of a lymphocyte of the invention expresses a CAR comprising an extracellular domain comprising or consisting of
(A) the amino acid sequence of SEQ ID NO:15, SEQ ID NO:16, SEQ ID NO:17, or SEQ ID NO:18;
(B) an amino acid sequence that is e.g. at least 85% identical to the amino acid sequence of SEQ ID NO:15, SEQ ID NO:16, SEQ ID NO:17, or SEQ ID NO:18 characterized by specifically binding CSF1R and having a c-myc tag; or
(C) a fragment of the amino acid sequence of (i) or (ii) characterized by specifically binding CSF1R and having a c-myc tag.

The extracellular domain of this example further (i) does not comprise an antibody Fc region or portion thereof, and/or (ii) does not have binding activity for one or more Fc receptors.

The CAR of the invention comprises, in addition to the extracellular domain, a transmembrane domain. The transmembrane domain can be any transmembrane domain known in the art or described herein suitable for use in such in a recombinant CAR, e.g. suitable for use in a signaling protein which signal is elicited by binding of the extracellular domain to the target antigen. Suitable transmembrane domains can be readily selected by the skilled person based on routine methods and knowledge in the art. Exemplary transmembrane domains include the murine CD28 transmembrane domain having an amino acid sequence of SEQ ID NO:19 (which may be, for example, encoded by SEQ ID NO:20) and the human CD28 transmembrane domain having the amino acid sequence of SEQ ID NO:21 (which may be, for example, encoded by SEQ ID NO:22). It is most preferred that the transmembrane domain be of human origin, e.g. the human CD28 transmembrane domain (SEQ ID NO:21).

The CAR of the invention comprises, in addition to the extracellular domain and transmembrane domain, also an intracellular domain having T cell activating activity. The intracellular domain having T cell activating activity (alternately referenced as an intracellular T cell activating domain) may comprise one or more stimulatory domains that transduce the signals necessary for lymphocyte (e.g. T cell) activation on the binding of the extracellular region to target antigen. Such cytoplasmic signaling domains are known in the art and include, for example, but not limited to, the intracellular signaling domain of CD3ζ, CD28, 4-1BB, OX40, as well as combinations thereof. The intracellular T cell activating domain of the CAR as described herein, or of the CAR recombinantly expressed by the lymphocyte as described herein, preferably comprises the signaling domain of the human CD3ζ chain and/or at least one costimulatory domain that is an intracellular domain of a human endogenous T cell receptor. Such a costimulatory domain may be an intracellular domain of at least CD28 but is not limited to this specific example. The intracellular signaling domain may comprise multiple costimulatory domains, for example not only including the signaling domains of the CD3ζ chain and CD28, but also of, e.g. CD137(4-1BB) as is known in the art. Most preferably, the intracellular T cell activating domain of the CAR as described herein or the CAR expressed by the lymphocyte as described herein comprises the signaling domain of the human CD3ζ chain and a costimulatory domain comprising an intracellular domain of human CD28 as is known in the art. Sequences of the signaling domain of the human CD3ζ chain are known and include, but are not limited to SEQ ID NO:33 (which may be encoded by SEQ ID NO:34); similarly sequences of suitable human CD28 co-stimulatory domains are also known and include, but are not limited to SEQ ID NO:35 (which may be encoded by SEQ ID NO:36).

As described herein above, the CAR of the invention, or the CAR recombinantly expressed by the lymphocyte as described herein (i.e. for use in the treatment of cancer characterized by the expression of CSF1R), most preferably comprises
(A) an extracellular domain comprising
   (i) an antigen binding region that is a human or humanized scFv specific for CSF1R, a variant amino acid sequence of such human or humanized scFv specific for CSF1R, or a fragment of such scFv or variant sequence which fragment is specific for CSF1R; and
   (ii) a spacer comprising a human hinge region and a detection/purification tag;
(B) a human transmembrane domain; and
(C) an intracellular domain comprising a human intracellular T cell activating domain and at least one human co-stimulatory domain that transduces the signals necessary for lymphocyte (e.g. T cell) activation on the binding of the extracellular region to target antigen.

Non-limiting examples of such a CAR comprise or consist of the amino acid sequence of SEQ ID NO:23 or SEQ ID NO:24. The CAR as described herein or the CAR expressed by the lymphocyte as described herein alternatively comprises or consists of a variant amino acid sequence of SEQ ID NO:23 or SEQ ID NO:24. Such a variant amino acid sequence can be an amino acid sequence variant polypeptide having an amino acid sequence that is at least 85%, 86%, 87%, 88%, 89%, 90%, 91%, 92%, 93%, 94%, 95%, 96%, 97%, 98% or 99% identical to SEQ ID NO: 23 or SEQ ID NO:24, provided that the sequence variant is characterized by specifically binding to CSF1R, exhibits the c-myc tag and exhibits T cell activating activity on binding to CSF1R. As is known in the art, T cell activating activity can be tested through release of pro-inflammatory cytokines (e.g. IFNy, IL-2, TNFalpha or GM-CSF) or the up-regulation of T cell activation or exhaustion markers (including but not limited to CD69, CD25, 4-1BB, CD28, PD-1, LAG-3 and Tim3) in response to CSF1R binding. Furthermore, the CAR as described herein or the CAR expressed by the lymphocyte as described herein may also comprise or consist of a fragment of the amino acid sequence of SEQ ID NO: 23 or SEQ ID NO:24 or of a fragment of the variant amino acid sequence of SEQ ID NO: 23 or SEQ ID NO:24, provided that such fragment is characterized by specifically binding to CSF1R, by exhibiting the c-myc tag and by exhibiting T cell activating activity on binding to CSF1R. Accordingly, the CAR as described herein or the CAR expressed by the lymphocyte for use in the treatment of cancer characterized by the expression of CSF1R as described herein, may comprise or consist of (i) the amino acid sequence of SEQ ID NO:23 or SEQ ID NO:24, or (ii) a SEQ ID NO:23 or SEQ ID NO:24 variant amino acid sequence, which variant sequence is at least 85% identical to SEQ ID NO:23 or SEQ ID NO:24 or (iii) a fragment of the amino acid sequence of SEQ ID NO:23 or SEQ ID NO:24 or a fragment of the SEQ ID NO:23 or SEQ ID NO:24 variant amino acid sequence, provided that the SEQ ID NO:23 or SEQ ID NO:24 variant amino acid sequence or fragment thereof is characterized by specifically binding to CSF1R, by exhibiting the c-myc tag and by exhibiting T cell activating activity on binding to CSF1R.

The person of skill in the art recognizes that transgenic proteins to be localized in the cell membrane require a signaling sequence for membrane localization, which signaling sequence is encoded by a nucleic acid sequence operably linked to the nucleic acid sequence encoding the membrane protein. The signaling sequence is translated together with the membrane protein, but is subsequently cleaved in post-translational processing. Such signal proteins are well known in the art, and can be suitable selected to localize, e.g. CARS comprising SEQ ID NO:23 or SEQ ID NO:24, their variant sequences and/or functional fragments as explained herein to the membrane using routine and standard methods. Exemplary amino acid sequences comprising SEQ ID NO:23 or SEQ ID NO:24 together with suitable membrane localization sequences include SEQ ID NO:25 and SEQ ID NO:27 (which may be encoded, for example, by SEQ ID NO:26 and SEQ ID NO:28, respectively).

In the context of the present invention, it is understood that the specific binding to CSF1R may be any binding that is accomplished between the target protein CFS1R, and the CAR as described herein, or between the target protein CSF1R and the lymphocyte recombinantly expressing the CAR as described herein. It is furthermore understood that the T cell activating activity on binding to CSF1R as described herein is provided by the CAR or the lymphocyte recombinantly expressing the CAR. Preferably, the CAR as disclosed herein is characterized in that (i) the specific binding is considered to be the specific binding of a lymphocyte recombinantly expressing the CAR to CSF1R; and/or (ii) that the T cell activating activity on binding to CSF1R is determined in a lymphocyte recombinantly expressing the CAR as described herein. The specific binding as well as the T cell activating activity on binding to CSF1R may be determined by methods known in the art and/or as described herein. Non-limiting examples of suitable methods are further disclosed in Xu et. al. (Methods Mol Biol. (2020); 2108:159-165).

The present invention provides a lymphocyte recombinantly expressing a CAR for use in the treatment of cancer. Accordingly, provided is a genetically engineered lymphocyte for use as a medicament. As is appreciated, CSF1R may or may not be expressed by the cancer cells (i.e., the diseased cells) themselves. A cancer also remains characterized by the expression of CSF1R where it is not expressed by the cancerous or diseased cells themselves, but where it is expressed by cells resident within the cancer/disease parenchyma and which are not cancer or disease cells. Such cells resident in the cancer/tumor/disease parenchyma that are not disease cells but that may express CSF1R include, but are not limited to, tumor resident immune cells or tumor infiltrating immune cells such as macrophages.

The cancer characterized by the expression of CSF1R targeted by the lymphocyte expressing the CAR as described herein may be any cancer including solid tumors known in the art such as breast cancer or pancreatic cancer, but is preferably a hematologic cancer. Hematologic cancers are understood in the art as cancers that initiate in blood-forming tissue, such as the bone marrow, or in the cells of the immune system. Non-limiting examples thereof include leukemia, lymphoma, and multiple myeloma. It is furthermore considered that precancerous disorders such as “preleukemic” blood disorders including myelodysplastic syndrome (MDS) or myeloproliferative neoplasms (MPN), are encompassed by the invention.

The present inventors have identified CSF1R as a ubiquitous target characterizing acute myeloid leukemia (AML). Accordingly, the cancer characterized by the expression of CSF1R targeted by the lymphocyte recombinantly expressing the CAR as described herein is most preferably acute myeloid leukemia (AML). The skilled person will recognize that the term AML as used herein encompasses refractory AML and relapsed AML. Furthermore, the present invention also encompasses the treatment of AML using the lymphocyte expressing the CAR as described herein, and/or using one or more CSF1R targeting agents. Such a targeting agent may be any molecular entity which specifically interferes with, targets, and/or binds to CSF1R expressed by the cancer, tumor resident cells, or cancer/disease parenchyma. Examples of such targeting agents include but are not limited to small molecule inhibitors targeting the CSF1R downstream signaling (Denny and Flanagan, Expert Opin Ther Pat. (2021); 31(2):107-117), and CSF1R-blocking antibodies (Cassier et al., Lancet Oncol. (2015); 16(8):949-56). Accordingly, the treatment of AML can be effected by the lymphocyte expressing the CAR alone, by the one or more CSF1R targeting agents alone, or by a combination thereof. As understood in the art, such combination treatment includes administering the CAR lymphocyte of the invention concurrently with the one or more additional CSF1R targeting agents as well as administration of the CAR lymphocyte prior or subsequent to the one or more additional CSF1R targeting agents. When administered concurrently, they may be in the same or different preparations.

The invention further provides a polynucleotide encoding the CAR as described herein. The skilled person is familiar with the expression “polynucleotide”, which generally relates to a nucleic acid sequence/nucleic acid molecule and is furthermore defined herein in detail. In the present invention, the polynucleotide encoding the CAR as disclosed herein can be part of a vector but is not limited thereto. The present invention therefore also relates to a vector comprising such a polynucleotide encoding the CAR as described herein. As will be readily appreciated by those skilled in the art, the vector is used as vehicle to artificially carry the genetic material encoding the CAR of the invention into a host cell. The skilled person is familiar with suitable vectors and in the foregoing detailed description of the invention, non-limiting examples thereof are further disclosed. Accordingly, the vector comprising a polynucleotide as described herein (i.e. encoding the CAR of the invention) may be any suitable vector, however, in the present invention, the vector is preferably a retroviral vector, an expression vector or a retroviral expression vector. As such, the vector comprising a polynucleotide encoding the CAR of the invention is preferably (i) a retroviral vector, and/or (ii) an expression vector.

Both the polynucleotide as well as the vector described herein are recognized as nucleic acid molecules which preferably replicate autonomously in a host cell (e.g. in a transduced cell) into which it has been introduced. Therefore, the invention further relates to a host cell comprising the polynucleotide encoding the CAR or the vector comprising such a polypeptide as disclosed herein, whether a lymphocyte (e.g. T cell) or not. As is known in the art, cells other than lymphocytes may be suitably be used as host cells comprising the nucleic acid and/or vector of the invention for the purpose, e.g. of amplifying the nucleic acids and/or vectors. Any cell suitable for genetic modification (i.e. introduction of a nucleic acid molecule into the cell so that it will express or maintain the introduced sequence/molecule) may be used as host cell. Suitable host cells are known in the art and include primary cells as well as cell lines. The host cell comprising the polynucleotide or the vector as disclosed herein is preferably a lymphocyte. Accordingly, the present invention further provides a host cell comprising the polynucleotide or the vector (comprising the polynucleotide) as described herein, which is a lymphocyte expressing the CAR as described herein.

The CAR as disclosed herein, a host cell comprising the polynucleotide or the vector encoding such a CAR, and/or the lymphocyte recombinantly expressing the CAR, their use, as well as the methods for their production are provided not only as therapeutic tools but will also be understood to have applicability as model systems for investigating disease therapies. As such, a lymphocyte in the context of the present invention may be any lymphocyte known in the art, known or believed to be of use in an in vitro or in vivo model system. Accordingly, although the lymphocyte recombinantly expressing the CAR as described herein (i.e. for use in the treatment of cancer characterized by the expression of CSF1R), and/or host cell is preferably a human lymphocyte as described herein, the invention encompasses host cells, e.g. lymphocytes of other mammalian species known to be of use in model systems, including but not limited to cells of rodent, canine, feline, porcine, caprine, ovine and primate origin. More preferably, the cells of the invention are primary human lymphocytes (e.g. including but not limited to NK cells and T cells), and most preferably primary human T cells (e.g. including but not limited to CD3+ T cell, a CD8+ T cell, a CD4+ T cell, a γδ T cell, an invariant T cell or a NK T cell). The invention also encompasses induced pluripotent stem cell (iPSC)-derived T cells, genetically engineered lymphocytes that are derived from lymphocyte cell lines (whether of human or non-human origin) and genetically engineered lymphocytes that are primary cells of human or non-human origin.

The lymphocytes of the invention recombinantly expressing the CAR of the invention may either be a directly genetically engineered lymphocyte, i.e. a lymphocyte that has been directly subjected to genetic engineering methods, or may be a lymphocyte derived from such a lymphocyte, e.g. a daughter cell or progeny of a lymphocyte that was directly genetically engineered. Thus, the genetically engineered lymphocyte of the invention may be a directly genetically engineered lymphocyte as well as any cell derived therefrom, such as a daughter cell obtained by culture of the directly engineered/modified lymphocyte.

Lymphocytes recombinantly expressing the CAR as described herein (i.e. for use in the treatment of cancer characterized by the expression of CSF1R) are envisioned for use in therapy and may be a lymphocyte autologous to the patient to be treated (i.e. the donor from which the cells were derived and recipient are the same subject) or alternatively a lymphocyte allogenic to the patient to be treated (i.e. the donor from which the cells were derived is different from the recipient). Where the cells are allogenic, they may be further genetically engineered or prepared such that they are not alloreactive. As understood in the art, and as used herein, not alloreactive indicates that the lymphocytes have been engineered (e.g., genetically engineered) such that they are rendered incapable of reacting to/recognizing allogenic (foreign) cells (in particular, other than those expressing the target antigen of the CAR). Similarly, the genetically engineered lymphocytes of the invention can be additionally or alternatively engineered so as to prevent their own recognition by the recipient's immune system. Lymphocytes can be rendered non-alloreactive and/or incapable of eliciting or being recognized by an immune system by any means known in the art or described herein. In the present invention, non-alloreactive cells can comprise genetic modifications to reduce or eliminate expression of the endogenous T cell receptor (TCR) genes or the endogenous TCR. Specifically, the lymphocyte or host cell recombinantly expressing the CAR as described herein (i.e. for use in the treatment of cancer characterized by the expression of CSF1R) may further be genetically engineered to reduce or eliminate expression of the endogenous T cell receptor (TCR) alpha or beta chain genes, or exhibits reduced or eliminated expression of the endogenous TCR. The genetic modification to the lymphocyte to reduce or eliminate alloreactivity and/or to reduce or eliminate self-antigen presentation as known in the art or as described herein, e.g. the reduction or elimination of expression of the endogenous T cell receptor (TCR) alpha or beta chain genes, or of the endogenous TCR can be performed before, concurrently with, or subsequent to the genetic engineering to express the CAR as described herein.

The lymphocytes or host cell recombinantly expressing the CAR as disclosed herein may also be genetically engineered to further express recombinant constructs including a dominant negative receptor (DNR), CD40-CD40L, KO Lag3, Tim3, PD-1, CTLA-4 or desired fusion receptors but not limited thereto. Specifically, the lymphocytes or host cell recombinantly expressing the CAR as disclosed herein may also be genetically engineered to further recombinantly express an exogenous cytokine receptor which may be adjuvant, e.g. in selecting, maintaining, expanding or stimulating the desired (primary) cell/cell population. Examples include interleukin-2 receptor (IL-2R), interleukin-7 receptor or interleukin-15 (IL-15R) (see, e.g., Dudley, Immunother. 26(2003), 332-342; Dudley, Clin. Oncol. 26(2008), 5233-5239). Accordingly, the lymphocytes of the present invention (expressing the CAR of the invention and for the use of the invention) may be further genetically modified according to none, one, two or all of the following: modified to reduce or eliminate expression of the endogenous T cell receptor (TCR) alpha or beta chain genes; modified to exhibit reduced or eliminated expression of the endogenous TCR; modified to recombinantly express an exogenous cytokine receptor; modified to reduce or eliminate alloreactivity; and/or modified so that it does not elicit an immune response or cannot be recognized by the recipient's immune system. These further modifications may occur before, concurrently with or subsequent to the genetic engineering in connection with expression of the CAR as described herein. As used herein the terms “does not elicit an immune response”, “cannot be recognized by the recipient's immune system”, “immunologically neutral” and/or analogous terms are not to be understood as absolutes. Cells engineered for such activity (or lack of activity) may exhibit some immunologic activating/stimulating activity, but at reduced levels relative to the levels of a control cell prior to the relevant modifications, e.g. genetic engineering. Inhibition of immune stimulatory activity or determination of immune response can be performed according to any method known in the art or described herein.

The present invention further provides a method for the production of a lymphocyte expressing the CAR as described herein. The lymphocyte to be produced may be the lymphocyte of the invention recombinantly expressing a CAR for use in the treatment of cancer characterized by the expression of CSF1R as disclosed herein, or may be the host cell comprising the genetic information to express the CAR as disclosed herein. In the present invention, the method for production comprises the steps of (i) introducing into the lymphocyte or host cell the polynucleotide encoding the CAR or the vector comprising the polynucleotide encoding CAR (e.g. an expression vector), (ii) culturing the lymphocyte or host cell recombinantly engineered according to (i) under conditions allowing the expression of the CAR; and (iii) recovering the engineered lymphocyte or host cell.

Accordingly, the invention also encompasses a genetically engineered lymphocyte or host cell expressing the CAR of the invention obtainable by the methods as disclosed herein. In this respect, the methods disclosed herein also encompass methods for expanding lymphocytes or host cells after the genetic engineering for expression of the CAR (and optional further genetic modifications as disclosed herein) as well as lymphocytes and host cells obtained after such expansion. The genetically engineered lymphocytes or host cells may be expanded by any suitable method known in the art or described herein. In the preferred embodiments wherein the cells or host cells are lymphocytes, non-limiting examples of methods accomplishing such expansion include exposure to one or more of the following: exposure to anti-CD3 antibodies, to anti-CD-28 antibodies, and to one or more cytokines. Where the lymphocyte is a T cell (e.g. a human T cell and most preferably a primary human T cell), it is preferred that the expansion is be performed at least by exposure to one or more suitable cytokines such as interleukin-2 (IL-2) and/or interleukin-15 (IL-15).

The present invention further provides the genetically engineered lymphocyte recombinantly expressing the CAR or the lymphocyte obtainable by the method as disclosed herein within a pharmaceutically acceptable carrier in the form of a pharmaceutical composition. Such pharmaceutical compositions are considered particularly useful in adoptive immune therapies. Where a pharmaceutical composition as disclosed herein comprises genetically engineered lymphocytes allogenic to the subject to be treated, such lymphocytes can be further genetically modified to be non-alloreactive and/or incapable of being recognized by the recipient's immune system as is known in the art or described herein. As described previously, such lymphocytes may be further genetically modified to reduce or eliminate expression of the endogenous T cell receptor (TCR) alpha or beta chain genes, or to exhibit reduced or eliminated expression of the endogenous TCR, and/or may be modified to recombinantly express an exogenous cytokine receptor.

The invention also relates to the following items:
Item [1] A lymphocyte recombinantly expressing a chimeric antigen T cell receptor (CAR) for use in the treatment of cancer characterized by the expression of colony stimulating factor 1 receptor (CSF1R), wherein said CAR comprises an extracellular domain that specifically binds CSF1R, a transmembrane domain, and an intracellular T cell activating domain.
Item [2] The lymphocyte for use according to item [1], wherein said extracellular domain comprises an antigen binding region specific for said CSF1R, and a spacer comprising a hinge region which connects said antigen binding region to the transmembrane domain of said CAR and which spacer or said hinge region
   (i) does not comprise an antibody Fc region or portion thereof, and/or
   (ii) does not have binding activity for one or more Fc receptors.
Item [3] The lymphocyte for use according to item [1] or [2], wherein said extracellular domain or said antigen binding region comprises an antigen single chain variable domain (scFv) specific for CSF1R.
Item [4] The lymphocyte for use according to item [3], wherein said scFv is a humanized or human scFv specific for CSF1R.
Item [5] The lymphocyte for use according to any one of items [2] to [4], wherein said spacer comprises a detectable tag allowing the detection of said lymphocyte, the purification of said lymphocyte, and/or the detection of said expressed CAR.
Item [6] The lymphocyte for use according to item [5], wherein said detectable tag is a c-myc tag.
Item [7] The lymphocyte for use according to any one of items [2] to [6], wherein said spacer or hinge region does not have binding activity for one or more Fc receptors, which one or more Fc receptor is an FcγR or FcRn.
Item [8] The lymphocyte for use according to any one of items [2] to [7] wherein said hinge region is a CD8 hinge region.
Item [9] The lymphocyte for use according to any one of items [2] to [8], wherein said hinge region is a human CD8 hinge region.
Item [10] The lymphocyte for use according to any one of items [1] to [9], wherein said extracellular domain comprises
   (i) an antigen binding region that is a human or humanized scFv specific for CSF1R; and
   (ii) a spacer comprising a human CD8 hinge region and a c-myc tag.
Item [11] The lymphocyte for use according to any one of items [1] to [10], wherein said intracellular T cell activating domain comprises the signaling domain of the CD3ζ chain and/or at least one costimulatory domain that is an intracellular domain of an endogenous T cell receptor.
Item [12] The lymphocyte for use according to item [11], wherein said costimulatory domain comprises an intracellular domain of at least CD28 and/or CD137(4-1BB).
Item [13] The lymphocyte for use according to any one of items [1] to [12], wherein said CAR comprises or consists of
   (a) the amino acid sequence of SEQ ID NO:23 or SEQ ID NO:24;
   (b) an amino acid sequence that is at least 85% identical to SEQ ID NO:23 or SEQ ID NO:24 (a SEQ ID NO:23 or SEQ ID NO:24 variant amino acid sequence), wherein said SEQ ID NO:23 or SEQ ID NO:24 variant amino acid sequence is characterized by specifically binding to CSF1R, by having a c-myc tag and further characterized by having T cell activating activity on binding to CSF1R; or
   (c) a fragment of the amino acid sequence of (a) or (b), wherein the fragment is characterized by specifically binding to CSF1R, by having a c-myc tag and further characterized by having T cell activating activity on binding to CSF1R.
Item [14] The lymphocyte for use according to any one of items [1] to [13], wherein said cancer is a hematological cancer.
Item [15] The lymphocyte for use according to any one of items [1] to [14], or a CSF1R targeting agent for use in the treatment of acute myeloid leukemia (AML).
Item [16] The lymphocyte for use according to any one of items [1] to [15], which lymphocyte is autologous to the patient to be treated.
Item [17] The lymphocyte for use according to any one of items [1] to [15], which lymphocyte is allogenic to the patient to be treated.
Item [18] A CAR comprising an extracellular domain that specifically binds CSF1R, a transmembrane domain, and an intracellular T cell activating domain, said extracellular domain comprising
   (i) an antigen binding region that is a human or humanized scFv antigen binding region specific for CSF1R; and
   (ii) a spacer comprising a human hinge region and a detectable tag allowing the detection and/or purification of said CAR or a cell expressing said CAR which spacer does not comprise an antibody Fc region or portion thereof and/or does not have binding activity for one or more Fc receptors.
Item [19] The CAR according to item [18], wherein the hinge region is a human CD8 hinge region and/or wherein said one or more Fc receptor is Fcγ or FcRn.
Item [20] The CAR according to item [18] or [19], wherein said detectable tag is a c-myc tag.
Item [21] The CAR according to any one of items [18] to [20], wherein said intracellular T cell activating domain comprises the signaling domain of the CD3ζ chain and/or at least one costimulatory domain that is an intracellular domain of an endogenous T cell receptor.
Item [22] The CAR according to item [21], wherein said costimulatory domain comprises an intracellular domain of at least CD28 and/or CD137(4-1BB).
Item [23] The CAR according to any one of items [18] to [22], wherein said CAR comprises or consists of
   (a) the amino acid sequence of SEQ ID NO:23 or SEQ ID NO:24;
   (b) an amino acid sequence that is at least 85% identical to SEQ ID NO:23 or SEQ ID NO:24 (a SEQ ID NO:23 or SEQ ID NO:24 variant amino acid sequence), wherein said SEQ ID NO:23 or SEQ ID NO:24 variant amino acid sequence is characterized by specifically binding to CSF1R, by having a c-myc tag and further characterized by having T cell activating activity on binding to CSF1R; or
   (c) a fragment of the amino acid sequence of (a) or (b), wherein the fragment is characterized by specifically binding to CSF1R, by having a c-myc tag and further characterized by having T cell activating activity on binding to CSF1R.
Item [24] The CAR according to item [23], wherein
   (a) said specific binding is the specific binding of a lymphocyte recombinantly expressing said CAR to CSF1R; and/or
   (b) said T cell activating activity is determined in a lymphocyte recombinantly expressing said CAR.
Item [25] A polynucleotide encoding the CAR according to any one of items [16] to [24].
Item [26] A vector comprising the polynucleotide of item [25].
Item [27] The vector of item [26] that is (a) a retroviral vector, and/or (b) an expression vector.
Item [28] A host cell comprising the polynucleotide according to item [25] or the vector according to item [26] or [27].
Item [29]. The host cell according to item [28] that is a lymphocyte expressing said CAR.
Item [30] The lymphocyte for use according to any one of items [1] to [17], or the lymphocyte according to item [29], wherein said lymphocyte is a human lymphocyte.
Item [31] The lymphocyte for use according to item [30], or the lymphocyte according to item [30], wherein said human lymphocyte is a primary human lymphocyte.
Item [32] The lymphocyte for use according to any one of items [1] to [17], [30] and [31], or the lymphocyte according to any one of items [29] to [31], wherein said lymphocyte is a T cell, NK cell or innate lymphoid cell.
Item [33] The lymphocyte for use according to item [32], or the lymphocyte according to item [32], wherein said T cell is a CD3+ T cell, a CD8+ T cell, a CD4+ T cell, a γδ T cell, an invariant T cell or a NK T cell.
Item [34] The lymphocyte for use according to any one of items [1] to [17] and [29] to [33], or the lymphocyte according to any one of items 29 to 33, wherein said lymphocyte is non-alloreactive.
Item [35] The lymphocyte for use according to item [34], or the lymphocyte according to item [34], wherein said lymphocyte is a T cell comprising genetic modifications to reduce or eliminate expression of the endogenous T cell receptor (TCR) alpha or beta chain genes, or exhibits reduced or eliminated expression of the endogenous TCR.
Item [36]. The lymphocyte for use according to any one of items [1] to [17] and [29] to [35], or the lymphocyte according to according to any one of items [29] to [35], further recombinantly expressing an exogenous cytokine receptor.
Item [37] A method for the production of the lymphocyte for the use according to any one of items [1] to [17] and [29] to [36], or the lymphocyte according to any one of items [29] to [36], comprising
   (a) introducing into the lymphocyte the polynucleotide according to item [25] or the vector according to item [26] or [27] that is an expression vector;
   (b) culturing the lymphocyte recombinantly engineered according to (a) under conditions allowing the expression of the CAR; and
   (c) recovering the engineered lymphocyte.
Item [38] The method according to item [37], wherein said lymphocyte is expanded after said recombinant engineering by exposure to one or more of
   (a) an anti-CD3 antibody;
   (b) an anti-CD28 antibody; and
   (c) one or more cytokines.
Item [39] The method according to item [38], wherein said lymphocyte is a T cell that is expanded at least by exposure to one or more cytokines, which one or more cytokines is at least interleukin-2 (IL-2) or interleukin-15 (IL-15).
Item [40] A lymphocyte recombinantly engineered to express the CAR according to any one of items [18] to [24] obtainable by the method according to any one of items [37] to [39].
Item [41] A pharmaceutical composition comprising the lymphocyte according to any one of items [29] to [36] and [40].

### 2. BRIEF DESCRIPTION OF FIGURES

FIG. 1: (A) Colony Stimulating Factor 1 Receptor (CSF1R) transcriptional expression in samples of human acute myeloid leukemia (AML) patients when compared to samples from healthy human bone marrow donors as determined by Gene Expression Profiling Interactive Analysis (GEPIA). (B) CSF1R expression in different AML subsets determined using BloodSpot database. Each individual patient is depicted as one dot (n=821 patients). p-values are based on two-sided unpaired t-test. The significance was considered as: p<0.05 (*), p<0.01 (**), p<0.001 (***) and p<0.0001 (****) for all comparisons.
HBM, healthy bone marrow; MDS, myelodysplastic syndrome;
AML-associated chromosomal aberrations: AML MLL, MLL-rearranged leukemia, AML inv(16), AML inversion 16, AML t(15,17), PML/RARalpha, AML t(8;21), RUNX1-RUNXIT1

FIG. 2: CSF1R expression in comparison to well-described AML-associated antigens IL3RA (CD123) or CD33 using single cell sequencing. (A) Uniform Manifold Approximation and Projection (UMAP) plot of pooled sequencing data from 16 different AML patients after sequencing a total of 30.712 cells.

FIG. 3: Expression of CSF1R determined by FACS analysis. (A) CSF1R expression on AML cell lines THP-1, Mv4-11, OCI-AML3, PL-21, MOLM-13, U937. B cell lymphoma cell line SU-DHL-4 was used as negative control. Representative FACS plot of at least three independent experiments is shown. Each cell line is depicted with two separate plots. Black line indicates antibody staining (upper graph) and light grey line indicates isotype control (lower graph). (B) Percentage of CSF1R+ cells on primary AML samples compared to an isotype control. Pooled results from a total of 7 patients are depicted.

FIG. 4: Effect of anti-CSF1R-CAR T cells on AML cell lines. GFP-expressing AML cell lines THP-1, MV4-11, OCI-AML and PL-21 were cocultured with transduced T cells expressing anti-CSF1R-CAR. Non-transduced T cells (NT) and mono-cultures of AML cell lines were used as control. (A) T cell activation as determined by INF-γ release quantified by ELISA, shown are representative results of three independent experiments. (B) T cell proliferation was determined by quantification of CD3+ cells by FACS analysis. Effector:target cell ratio was applied as indicated (one representative of three independent experiments). (C) Therapeutic effectivity of anti-CSF1R-CAR T cells was assessed by determining AML tumor cell lysis. GFP-positive cells were quantified using flow cytometry. Effector:target cell ratio was applied as indicated (one representative of three independent experiments is depicted). (D) Target specificity of anti-CSF1R-CAR T cells. Specificity of anti-CSF1R-CAR T cells was determined by coculturing transduced T cells expressing anti-CSF1R-CAR with CSF1R-negative non-Hodgkin lymphoma cells. Pooled data of 4 independent experiments is depicted. Transduced T cells expressing anti-CD19-CAR were used as positive control and untransduced (UT) T cells were used as negative control. Effector:target cells ratio was applied 0.2:1, 1:1, 5:1 and 10:1. Target cell lysis was determined by using BioGlo luciferase assay as described in the methods section. (A-C) p-values are based on two-sided unpaired t-test. The significance was considered as: p<0.05 (*), p<0.01 (**), p<0.001 (***) and p<0.0001 (****) for all comparisons.

FIG. 5: In vivo therapeutic efficacy of anti-CSF1R-CAR T cells in human xenograft AML mouse models. AML was established in mice by intravenous injection of the human AML cell line MV4-11 (A) or THP-1 (B) expressing luciferase. Transduced T cells expressing anti-CSF1R-CAR, anti-CD33-CAR a co-stimulation control-construct or PBS were intravenously injected after tumor establishment. (A) Treatment-induced immune response of MV4-11 tumor-bearing animals as determined by quantification of luminescence signal by in vivo imaging platform (IVIS, PerkinElmer, n=5 mice per group) (B) Survival of THP-1 bearing animals after treatment with anti-CSF1R CAR T cells, anti-CD33 CAR T cells, control-transduced T cells or PBS (n=10 mice per group). (C) Therapeutic effectivity of anti-CSF1R CAR T cells in Patient-derived Xenograft (PDX) models. AML was induced in recipient animals through i.v. injection of PDX-AML cells. Progression of leukemia was monitored using IVIS. Following establishment of AML in the recipient mice, animals were treated either with anti-CSF1R CAR T cells, anti-CD19 CAR T cells (negative control) or PBS. Shown is pooled data from a total of 5 mice per group. (A) p-values are based on two-way ANOVA (mixed-effects analysis). The significance was considered as: p<0.05 (*), p<0.01 (**), p<0.001 (***) and p<0.0001 (****) for all comparisons. (B) Statistical significance was calculated using log-rank test.

FIG. 6: Therapeutic effect of anti-CSF1R-CAR T cells on AML cell lines and primary AML blasts when compared to anti-CD33-CAR T cells. A) Transduced T cells expressing anti-CSF1R-CAR or anti-CD33-CAR were cocultured with AML cell lines THP-1, MV4-11, OCI-AML or PL-21 (n=5-9 different donors). Specific cell lysis was quantified using BioGlo Luciferase assay. Untransduced T cells (UT) were used as control. B and C) Transduced allogenic (B, n=10-13) or autologous (C, n=4) T cells expressing anti-CSF1R-CAR or anti-CD33-CAR were cocultured with primary blasts obtained from AML patients in effector:target (E:T) cells ratios as indicated in B. For autologous co-cultures (C) E:T ratio of 1:1 was used. Specific lysis of AML blasts was quantified by using FACS analysis. Untransduced T cells (UT) were used as control.

FIG. 7: (A-D) Expression of CSF1R on cells of hematopoietic lineage when compared to expression of CD33. Expression of CSF1R and CD33 was determined on CD34-positive hematopoietic stem cells (HSC), common myeloid progenitor cells (CMP), granulocyte/monocyte progenitor cells (GMP) and megakaryocyte/erythroid progenitor cells (MEP) using BloodSpot database. P-values are based on two-sided unpaired t-test.

FIG. 8: Expression of CSF1R on cells of hematopoietic lineage in comparison to CD33 and IL3RA using single cell sequencing. UMAP plots of pooled data from a total of 7.654 sequenced cells from 5 independent healthy donors are depicted.

FIG. 9: Expression of CSF1R or CD33 on CD34+ cord blood-derived hematopoietic stem cells (HSC) from healthy donors as determined by FACS analysis. HSC were stained after expansion for a total of 7 days as described in the methods section. Shown is one representative FACS plot of three independent experiments. A) Total frequency of CSF1R and CD33 expressing cells on live hematopoietic stem cells (identified after gating on fixable viability dye-negative cells). B) Expression of CSF1R and CD33 was determined on CD34- and CD38-positive progenitor cells (upper panel), and on CD34-positive, CD38-negative stem cells (lower panel).

FIG. 10: Target specificity of anti-CSF1R-CAR T cells when compared to anti-CD33-CAR T cells. A) Off-target killing of anti-CSF1R-CAR and anti-CD33-CAR T cells was determined by coculturing transduced T cells expressing anti-CSF1R-CAR or anti-CD33-CAR with bone marrow-derived CD34+ stem cells from healthy human donors. Shown are representative results from three independent experiments. Target cell lysis was determined by quantification of viable cells using flow cytometry. Untransduced T cells (UT) were used as control and effector:target cells ratio was applied as indicated. B-D) Activation and exhaustion of anti-CSF1R-CAR and anti-CD33-CAR T cells as determined by FACS analysis by coculturing transduced T cells expressing anti-CSF1R-CAR or anti-CD33-CAR with peripheral blood mononuclear cells (PBMC) from healthy human donors (n=4-9). Untransduced T cells (UT) were used as control. (A-D) p-values are based on two-sided unpaired t-test.

FIG. 11: Off-target killing of anti-CSF1R-CAR and anti-CD33-CAR T cells was determined by coculturing transduced T cells expressing anti-CSF1R-CAR or anti-CD33-CAR with bone marrow from healthy human donors (n=3-6). Target cell lysis was determined by FACS analysis. Effector:target cells ratio was applied as indicated and measurement was conducted after incubation for 3 or 6 days.

FIG. 12 Listing of Sequences

FIG. 13: Workflow of computational CAR target antigen identification by stepwise evaluation against a set of criteria for an ideal and effective CAR target antigen. A total of 12 different, publicly available scRNA-seq datasets were used for the analysis (544,764 sequenced single cells). Number of screened genes are illustrated at the bottom. scRNA-seq: single-cell RNA-sequencing; HSPC: hematopoietic stem and progenitor cells, CSPA: Cell surface protein atlas; HPA: Human protein atlas.

FIG. 14: Volcano plot showing CSF1R as one of the identified target antigens with its respective −log 10 p-value and log 2 fold changes from differential expression analysis between healthy and malignant HSPC. Dotted lines indicate applied thresholds at log 2fc=2 and p-value=0.01.

FIG. 15: Expression of CSF1R on primary AML blasts of AML cell lines over a defined time course directly after thawing determined by FACS analysis. (A) Percentage of CSF1R positive cells determined by flow cytometry over a time course of 72 hours directly after thawing of primary AML blasts. (B) Representative FACS plot of five different donors illustrating the change of expression over 72 hours. (A, B) Shown is data from 10 different patients. (C) Expression of CSF1R on four different AML cell lines (THP-1, Mv4-11, OCI-AML3 and PL-21) directly after thawing and after 24 hours. AMFI: Delta mean fluorescent intensity. Representative FACS plot of at least two independent experiments were depicted.

FIG. 16: In vivo therapeutic efficacy of anti-CSF1R-CAR T cells inhuman xenograft AML mouse models. AML was established in mice by intravenous injection of the human AML cell line OCI-AML3 expressing luciferase. Transduced T cells expressing anti-CSF1R-CAR or a co-stimulation control-construct were intravenously injected after tumor establishment. (A) Survival of OCI-AML3-bearing animals after treatment with anti-CSF1R CAR T cells or control-transduced T cells (n=5 mice per group). (B) Treatment-induced immune response of OCI-AML3-tumor-bearing animals as determined by quantification of luminescence signal by in vivo imaging platform (IVIS, PerkinElmer, n=5 mice per group). Shown is pooled data from a total of 5 mice per group. The significance was considered as: p<0.05 (*), p<0.01 (**), p<0.001 (***) and p<0.0001 (****) for all comparisons. Statistical significance was calculated using log-rank test (A) or two-way ANOVA (mixed-effects analysis) (B).

### 3. DETAILED DESCRIPTION

T cells are already established as major target structures and effectors in oncology (Kobold et al., 2015), and first clinical trials demonstrate that T cell-based therapies are a promising approach for the treatment of a variety of human diseases including malignant conditions. In the treatment of AML, CAR T cells as well as bispecific antibodies against CD33 are under investigation. However, they have been shown to yield clinically to severe side-effects (Wang et al.), likely due to a lack of specificity of CD33 as target structure. This further demonstrates the high demand of appropriate target structures for an effective T cell-based AML treatment.

The important role of colony stimulating factor 1 receptor (CSF1R) in the context of tumor-diseases and immunosuppression has recently been identified (Ries et al., 2014). CSF1R is a single pass type I membrane protein and acts as the receptor for the cytokine colony stimulating factor 1 (CSF1). CSF1R is known to be expressed in vivo on distinct myeloid subpopulation, and in the context of AML, an amplification of CSF1R signaling and the therapeutic potential of its inhibition has only been described for rare subtypes (Edwards et al., 2018). A broad expression of CSF1R and a broad application of this signaling pathways has been denied (Aikawa et al., 2020; Edwards et al., 2018) based on its putative low expression. As a result, CSF1R has not been considered as suitable target for the treatment of AML. In contrast, it is demonstrated herein that that CSF1R is a broadly expressed AML target structure that can be effectively used as a target molecule in ACT. Specifically, it is demonstrated that lymphocytes genetically engineered to express an anti-CSF1R chimeric antigen receptor (anti-CSF1R CAR), improve therapeutic efficacy in adoptive therapeutic strategies. The methods disclosed herein are applicable to any type of lymphocyte capable of being used in adoptive therapy, including, but not limited to, natural killer (NK) cells and T cells. T cells of use in accordance with the methods disclosed herein include, for example, CD4+ T cells, CD8+ T cells, and γδ T cells.

The present invention provides a lymphocyte recombinantly expressing a chimeric antigen T cell receptor (CAR) for use in the treatment of cancer characterized by the expression of colony stimulating factor 1 receptor (CSF1R). Accordingly, provided is a lymphocyte, preferably a human lymphocyte, more preferably a primary human lymphocyte and most preferably a primary human T cell, NK cell or innate lymphoid cell that has been genetically engineered to recombinantly express an anti-CSF1R CAR. The lymphocytes according to the present invention can be any lymphocyte described herein or known in the art to be suitable for use, in particular, in an adoptive cell therapy. As used herein the term “innate lymphoid cells” (ILCs) references a heterogenous group of cells comprising NK cells and non-cytotoxic ILCs. ILCs are understood as the innate system counterpart of T cells. Although ILCs lack a T cell receptor, they exhibit the capacity to induce cell death (e.g. by means of the TRAIL pathway) and secrete cytokines similarly to T cells. ILCs are subclassified into ILC1, ILC2, and ILC3 which share similarities with T cell subsets Th1, Th2 and Th17, respectively. ILCs are tissue resident cells than can rapidly respond to diverse environmental signals and show a remarkable plasticity. The plasticity and their ability to migrate to and reside within different tissues separately and/or in combination lead to their therapeutic advantages, e.g. for use in the treatment of solid tumors.

It is recognized that the lymphocytes (and furthermore the CAR and the methods of the invention) may also be applicable for uses outside of therapies, such as in screening methods and/or in model systems, e.g. of use in in vitro assays or in vivo animal models. Therefore, the invention also encompasses the use of non-human sequences in the development of the CARS, genetically engineered non-human lymphocytes and/or genetically engineered lymphocytes derived from cell lines or induced pluripotent stem cells (iPSC), which may be of human or non-human origin. Exemplary sequences that may be of use in this respect include hinge domains as explained herein of murine origin, e.g. a murine CD8 hinge domain comprising or consisting of SEQ ID NO:5 (which may be encoded, for example, by SEQ ID NO:6). Similarly, transmembrane and intracellular (T cell activation) sequences may also be used in this respect. Exemplary such sequences include murine transmembrane domains (such as a murine CD28 transmembrane domain (e.g. SEQ ID NO:19, which may be encoded by SEQ ID NO:20)), murine intracellular T cell activating domains (such as the intracellular T cell activation domain from murine CD3ζ (e.g. SEQ ID NO:29, which may be encoded by SEQ ID NO:30)), and murine intracellular T cell co-stimulatory domains (such as the stimulatory domain of murine CD28 (e.g. SEQ ID NO:31, which may be encoded by SEQ ID NO:32).

Where the lymphocytes are derived from iPSCs as noted above, the iPSCs may be originally derived from any suitable cell but are preferably developed into T cells (T-iPSCs). Non-limiting examples of lymphocytes (which may be primary lymphocytes or derived from cell lines or iPSCs) include NK cells, inflammatory T lymphocytes, cytotoxic T lymphocytes, helper T lymphocytes, CD4+ T lymphocytes, CD8+ T lymphocytes, γδ T lymphocytes, invariant T lymphocytes and NK T lymphocytes. It is preferred that the genetically engineered lymphocyte, i.e. the lymphocyte recombinantly expressing the CAR as described herein, is a genetically engineered human lymphocyte. Thus it is preferred that the cell of the invention is a genetically engineered human NK cell or T cell, more preferably a primary human NK or T cell, and most preferably a primary human T cell, which may be, e.g., a CD8+ T cell, a CD4+-T cell, or γδ T cell.

The term “primary” and analogous terms in reference to a cell or cell population as used herein correspond to their commonly understood meaning in the art, i.e., referring to cells that have been obtained directly from living tissue (i.e. a biopsy such as a blood sample) or from a subject, which cells have not been passaged in culture, or have been passaged and maintained in culture but without immortalization. It is more preferred that the engineered lymphocytes are engineered primary human lymphocytes. Primary cells have undergone very few population doublings, if any, subsequent to having been obtained from the tissue sample and/or subject, and are therefore more representative of the main functional components and characteristics of in situ tissues and cells as compared to continuous tumorigenic or artificially immortalized cell lines. The primary lymphocytes described herein can be isolated and/or obtained from a number of tissue sources, including but not limited to, peripheral blood mononuclear cells isolated from a blood sample, bone marrow, lymph node tissue, cord blood, thymus tissue, tissue from a site of infection, ascites, pleural effusion, spleen tissue, and/or tumors by any method known in the art or described herein. In a non-limiting example in the context of a T cell, a genetically engineered primary T cell of the present invention is that having been obtained and/or isolated from a T cell population from subject (preferably a human patient). Methods for isolating/obtaining specific populations of lymphocytes (including T cells) from patients or from donors are well known in the art and include as a first step, for example, isolation/obtaining a donor or patient sample known or expected to contain such cells, e.g., a blood or bone marrow sample. After isolating/obtaining the sample, the desired cells, e.g., NK cells or T cells, are separated from the other components in the sample. Methods for separating a specific population of desired cells from the sample are known and include, but are not limited to, e.g., leukapheresis for obtaining T cells from the peripheral blood sample from a patient or from a donor; isolating/obtaining specific populations from the sample using a FACSort apparatus; and selecting specific populations from fresh biopsy specimens comprising living lymphocytes by hand or by using a micromanipulator (see, e.g., Dudley et al., Immunother. (2003), (26):332-342; Robbins et al., Clin. Oncol. (2011), (29):917-924; Leisegang, J. Mol. Med. (2008), (86):573-58). The term “fresh biopsy specimens” refers to a tissue sample (e.g. a tumor tissue or blood sample) that has been or is to be removed and/or isolated from a subject by surgical or any other known means. The isolated/obtained cells are subsequently cultured and expanded according to routine methods known in the art for maintaining and/or expanding the desired primary cell and/or primary cell population. For example, in the context of T cells, culture may occur in the presence of an anti-CD3 antibody; in the presence of a combination of anti-CD3 and anti-CD28 monoclonal antibodies, and/or in the presence of an anti-CD3 antibody, an anti-CD28 antibody and one or more cytokines, e.g. interleukin-2 (IL-2) and/or interleukin-15 (IL-15) (see, e.g., Dudley et al., Immunother. (2003), (26):332-342; Dudley et al., Clin. Oncol. (2008), 26:5233-5239).

As is well known in the art, it is also possible to isolate/obtain and culture/select one or more specific sub-populations of lymphocytes or T cells, which methods are also encompassed by the invention. Such methods include but are not limited to isolation and culture of primary cell sub-populations, e.g. primary T cell sub-populations such as CD3+, CD28+, CD4+, CD8+, and γδ, as well as the isolation and culture of other primary lymphocyte populations such as NK T cells or invariant T cells. Such selection methods can comprise positive and/or negative selection techniques, e.g. wherein the sample is incubated with specific combinations of antibodies and/or cytokines to select for the desired sub-population. The skilled person can readily adjust the components of the selection medium and/or method and length of the selection using well known methods in the art. Longer incubation times may be used to isolate desired populations in any situation where there is or are expected to be fewer desired cells relative to other cell types, e.g. such as in isolating tumor infiltrating lymphocytes (TIL) from tumor tissue or from immunocompromised individuals. The skilled person will also recognize that multiple rounds of selection can be used in the disclosed methods. Enrichment of the desired population is also possible by negative selection, e.g. achieved with a combination of antibodies directed to surface markers unique to the negatively selected cells. In a non-limiting example, cell sorting and/or selection via negative magnetic immunoadherence or flow cytometry which use a cocktail of monoclonal antibodies directed to cell surface markers present on the cells negatively selected can be used. For example, to enrich CD4+ T cells by negative selection, a monoclonal antibody cocktail typically including antibodies specific for CD14, CD20, CD11b, CD16, HLA-DR, and CD8 is used. The methods disclosed herein also encompass removing T regulatory cells, e.g., CD25+ T cells, from the population to be genetically engineered. Such methods include using an anti-CD25 antibody, or a fragment thereof, or a CD25-binding ligand, such as IL-2.

The lymphocyte recombinantly expressing the CAR as described herein may be a genetically engineered autologous primary lymphocyte. The term “autologous” refers to any material isolated, derived and/or obtained from the same individual to whom it is later to be re-introduced, e.g. in the context of an autologous adoptive therapy, such as autologous adoptive T cell therapy (ACT) wherein the same individual is both the donor and recipient. Accordingly, in the context of the invention, the genetically engineered lymphocyte may be a genetically engineered autologous primary lymphocyte, including but not limited to a genetically engineered primary autologous NK cell or a primary autologous T cell, such as a primary autologous CD8+ T cell, a primary autologous CD4+ T cell, a primary autologous γδ T cell, a primary autologous invariant T cell or a primary autologous NK T cell. However, the methods and materials disclosed herein (e.g. the genetically engineered lymphocyte) are not limited to autologous lymphocytes isolated and/or derived from the subject to be subsequently treated with the lymphocytes (and/or are not limited to the use of such autologous lymphocytes, e.g. as a medicament in the treatment of a disease characterized by CSF1R). The methods disclosed herein also encompass the use and production of genetically engineered allogeneic lymphocytes, in particular primary lymphocytes. As appreciated in the art, an “allogeneic lymphocyte” is a lymphocyte (e.g. a T cell) isolated from a donor of the same species as the recipient but not genetically identical to the recipient. Such allogenic cells can be used in adoptive therapies without or, preferably, with further modification as described herein, e.g. to reduce or inactivate the allogenic reactions in the intended recipient by the engineered T cell to the host (e.g. graft versus host reactions) as well as those immune reactions of the host against the engineered T cell (e.g. host versus graft reactions). Such modifications can be made by any method known in the art and/or described herein (such cells are known in the art and referenced herein as “non-alloreactive” lymphocytes/T cells). As such, where the cells are allogenic, they may be further genetically engineered or prepared such that they are not alloreactive. As understood in the art, and as used herein, not alloreactive (or, alternatively, non-alloreactive) indicates that the lymphocytes/T cells have been engineered (e.g. genetically engineered) such that they are rendered incapable of reacting to/recognizing allogenic (foreign) cells other than the cells expressing the target antigen specifically bound/recognized by the antigen-binding region of the CAR of the invention. Therefore, non-alloreactive cells derived from third-party donors may become universal, i.e. recipient independent. Similarly, the genetically engineered lymphocytes of the invention can be additionally or alternatively engineered so as to rendering them incapable of eliciting an immune response and/or of being recognized by the recipient's immune system, preventing them from being rejected. Such cells that are non-alloreactive and/or that are incapable of eliciting an immune response or being recognized by the recipient's immune system may also be termed “off-the-shelf” lymphocytes as is known in the art. Lymphocytes can be rendered non-alloreactive and/or incapable of eliciting or being recognized by an immune system by any means known in the art or described herein. As a non-limiting example in this respect, the lymphocytes of the invention may have disruption or deletion of the endogenous major histocompatibility complex (MHC). Such cells may have diminished or eliminated expression of the endogenous MHC when compared to an unmodified control cell, preventing or diminishing activation of the recipient's immune system against the autologous cells. In the context of T cells, as a non-limiting example, non-alloreactive cells can have reduced or eliminated expression of the endogenous T cell receptor (TCR) when compared to an unmodified control cell. Such non-alloreactive T cells may comprise modified or deleted genes involved in self-recognition, such as but not limited to, those encoding components of the TCR including, for example, the alpha and/or beta chain. The genetic modifications to reduce or eliminate alloreactivity (i.e. to render the cell non-alloreactive other than against cells expressing the antigen of choice (i.e. that specifically bound by the antigen-binding region of the CAR of the invention)) and/or to reduce or eliminate self-antigen presentation (i.e. so as to prevent them from eliciting an immune response or being recognized by the recipient's immune system), as known in the art or described herein can be performed before, concurrently with, or subsequent to the genetic engineering to express the CAR as defined herein. As a non-limiting example, non-alloreactive/off the shelf lymphocytes can be obtained from a repository and then engineered to express the CAR of the invention according to the methods described herein and subsequently used in the treatment, in particular, of cancers characterized by CSF1R. In such comprising the use of “off-the-shelf” lymphocytes, the modifications to render the lymphocyte non-alloreactive and/or incapable of eliciting an immune response and/or being recognized by the recipient's immune system were performed prior to the genetic engineering to express the CAR.

The donor and/or recipient of the lymphocytes as disclosed herein, including the subject to be treated with the allogenic or autologous genetically engineered primary lymphocytes, may be any living organism in which an immune response can be elicited (e.g. mammals). Examples of donors and/or recipients as used herein include humans, dogs, cats, mice, rats, monkeys and apes, as well as transgenic species thereof, and are preferably humans.

As used herein, the term “recombinantly expressing” and analogous terms, refers to (i) a cell that has been recombinantly/genetically modified to express a CAR as described herein; as well as (ii) the progeny of such a cell that maintains expression of such a polypeptide, e.g., obtainable by culture of the originally modified cell. Methods of genetically engineering cells to express polypeptides of interest are well known and routine in the art and include methods of introducing nucleic acids encoding the polypeptide in an appropriate form (e.g. in an expression vector) into cells via chemical or viral means. Therefore, a cell “recombinantly expressing” a polypeptide according to the invention generally encompasses the deliberate introduction of a nucleic acid molecule into the cell so that it will express the introduced sequence/molecule to produce a desired substance, e.g. a CAR. “Recombinantly expressing” encompasses any means of introducing the nucleic acid sequence or molecule (e.g. a polynucleotide or vector) into the cell described herein or known in the art suitable to allow expression of the encoded polypeptide. Thus, “recombinantly expressing” encompasses transduction methods (commonly understood to refer to the introduction of a foreign nucleic acid into a cell using a vector, including the use of a viral vector), and transfection methods (commonly understood to refer to the introduction of a foreign nucleic acid into a cell using non-viral means such as chemical- or electric poration, microinjection, etc.). Thus, “recombinantly expressing” in more general terms also encompasses methods of transformation, i.e. the introduction of a gene, DNA or RNA sequence into a host cell, such that the host cell will express the introduced gene or sequence to produce a desired substance, such as a polypeptide (e.g. a CAR) encoded by the introduced gene or sequence (e.g. a polynucleotide sequence). The introduced gene or sequence can be referenced as a “cloned”, “foreign”, or “heterologous” gene or sequence, or a “transgene”. The introduced nucleic acid molecule/sequence can also comprise additional heterologous sequences including, for example, include heterologous promoters, start, stop, promoter, signal, secretion, or other sequences used by a cell's genetic machinery operatively linked to the coding sequences described herein, as well as further regulatory nucleic acid sequences well known in the art and/or described herein. The introduced gene or sequence can include nonfunctional sequences or sequences with no known function. According to the methods disclosed herein, a host cell that receives and expresses introduced DNA or RNA has been “genetically engineered”. As understood in the art, genetically engineered in the context of the methods and products described herein is equivalent to transformed, transduced and/or transfected, and the genetically engineered cell is, for example, a transformant or a clone and is “transgenic”. The DNA or RNA introduced to the host cell, i.e. the lymphocyte, can be derived from any source, including cells of the same genus or species as the host cell, or cells of a different genus or species.

The lymphocytes recombinantly expressing the CAR of the invention are preferably cultured under controlled conditions outside of their natural environment. In particular, the term “culturing” as used herein indicates that the engineered cells are maintained in vitro. The genetically engineered lymphocytes are cultured under conditions allowing the expression of the CAR as described herein. Conditions that allow the maintenance of lymphocytes and expression of a desired transgene therein are commonly known in the art and include, but are not limited to culture in the presence of agonistic anti-CD3- and anti-CD28 antibodies, as well as one or more cytokines such as interleukin 2 (IL-2), interleukin 7 (IL-7), interleukin 12 (IL-12) and/or interleukin 15 (IL-15). After expression of the CAR as described herein, the genetically engineered cell is recovered or otherwise isolated from the culture.

Accordingly, also provided herein is a method for the production of a lymphocyte recombinantly expressing the CAR as described herein (e.g. a human primary T cell), comprising the steps of modifying (e.g. transducing) the cell to express the CAR, culturing the modified/recombinant cell under conditions allowing the expression of the CAR, and recovering said genetically engineered cell. The lymphocytes as described herein may be activated and/or expanded as is known in the art. Thus, methods according to the invention may also include a step of activating and/or expanding a primary lymphocyte or lymphocyte population. This can be done prior to or after genetic engineering of the cells, using the methods well known in the art, e.g. as described in U.S. Pat. Nos. 6,352,694; 6,534,055; 6,905,680; 6,692,964; 5,858,358; 6,887,466; 6,905,681; 7,144,575; 7,067,318; 7,172,869; 7,232,566; 7,175,843; 5,883,223; 6,905,874; 6,797,514; 6,867,041; and U.S. Patent Application Publication No. 20060121005. As appreciated in the art, such methods can encompass culturing the cells with appropriate agents such as agents that activate stimulatory receptors (e.g. agonistic antibodies) and/or target ligands of endogenous or recombinant receptors as routine in the art. Said cells can also be expanded by co-culturing with tissue or cells expressing target ligands of endogenous or recombinant receptors, including in vivo, for example in the subject's blood after administrating the cells to the subject.

The lymphocyte recombinantly expressing the CAR (e.g. a primary T cell) provided herein may comprise a polynucleotide molecule, or a vector comprising the polynucleotide molecule, encoding the CAR as described herein. The CAR of the invention comprises an extracellular domain that specifically binds CSF1R, a transmembrane domain, and an intracellular T cell activating domain. As such, only a part of the receptor is accessible from the intracellular space. Once engineered into the lymphocyte(s), the encoded CAR (i.e. the extracellular part thereof) is expressed on the surface of the engineered cell and can be detected either directly, e.g., by flow cytometry or microscopy using antibodies specific for the CAR as described herein or a portion thereof (e.g. specific of the tag within the spacer of the extracellular domain, in particular, a c-myc tag) or indirectly, e.g., by assessing the engineered cells for anti-CSF1R activity by any method known in the art and/or described herein.

The extracellular domain of the CAR as described herein shows specific binding to CSF1R. In the context of the present invention, the term “binding to” is interchangeable with the term “interacting with” and “specific for” and not only relates to a linear epitope but may also relate to a conformational epitope, a structural epitope or a discontinuous epitope consisting of two regions of the, e.g. human, target molecules or parts thereof. Only CAR constructs that bind to the (poly)peptide/protein of interest, i.e. CSF1R, but that do not or do not essentially bind to any other (poly)peptide/protein expressed by the same tissue as the (poly)peptide/protein of interest, e.g. by the tumor cells, are considered specific for the (poly)peptide/protein of interest as is known and accepted in the art. Methods to determine binding may comprise, inter alia, binding studies, blocking and competition studies with structurally and/or functionally closely related molecules. Non-limiting examples of methods to assess specificity to CSF1R include Western Blots, ELISA-, RIA-, ECL-, IRMA-tests and peptide scans. Binding studies also comprise FACS analysis, surface plasmon resonance (SPR, e.g. with BIAcore), analytical ultracentrifugation, isothermal titration calorimetry, fluorescence anisotropy, fluorescence spectroscopy or by radiolabeled ligand binding assays. Furthermore, physiological assays like cytotoxic assays may be performed. Accordingly, examples for the specific interaction of an antigen-interaction-site with a specific antigen comprise the specificity of a ligand for its receptor or vice versa. Said definition particularly comprises the interaction of ligands which induce a signal upon binding to its specific receptor.

It is well known in the art that the term “specifically binds”, “recognizes”, “interacts with” and analogous terms designate the degree to which an antigen binding region discriminates between two antigens. This is because it is known that no antigen binding region, e.g. an antibody antigen binding region, has absolute specificity, in the sense that it will react with only one epitope whatever the conditions. That is, where other (non-target) antigens are present, an antigen binding region may react to some extent with similar epitopes on these other (non-target) antigens. However, the affinity of an antigen binding region for its target epitope/antigen is significantly greater than its affinity for related epitopes. This difference in affinity is used to establish assay conditions, under which an antigen binding region binds almost exclusively to a specific (target) epitope. In this respect, the binding (or non-binding) of an antigen binding region to an antigen are not understood as absolutes. That is, the CAR of the invention, the cell expressing a CAR of the invention, and/or the antigen-binding region of the CAR of the invention may exhibit some (residual) binding activity for other (non-)targets, but at significantly reduced levels relative to the binding activity for CSF1R. It is preferred that the antigen-binding domain of the CAR of the invention, the CAR and/or cell expressing the CAR exhibit at least 10 fold, at least 20 fold, preferably at least 50 fold, and more preferably at least 100 fold better affinity for CSF1R as compared to the affinity for a non-target antigen.

In a preferred aspect, the extracellular domain of the CAR of the invention comprises an antigen binding region specific for CSF1R and a spacer. The spacer is most preferably a peptide spacer which connects the antigen-binding region with the transmembrane domain of the CAR as described herein. Spacers offer the advantage of allowing the different domains/regions of the CAR (i.e. the antigen binding region and the transmembrane domain of said CAR) to fold independently and exhibit the expected activity. Thus, in the context of the present invention, the extracellular domain, the transmembrane domain and the intracellular T cell activating domain of the CAR may be comprised in a single-chain multi-functional polypeptide. In the present invention, the spacer as described herein (i) does not comprise an antibody/immunoglobulin Fc region or portion thereof (i.e. does not originate from an antibody/immunoglobulin Fc region or portion thereof) and/or (ii) does not have binding activity for one or more Fc receptors (FcR). The one or more Fc receptor may be a FcRn and/or an Fcγ receptor as known in the art or described herein, e.g., in humans the family includes FcγRI (CD64) including isoforms FcγRIa, FcγRIb and FcγRIc; FcγRII (CD32) including isoforms FcγRIIa (including allotype H131 and R131), FcγRIIb (including FcγRIIb-1 and FcγRIIb-2), and FcγRIIc; and FcγRIII (CD16) including isoform FcγRIIIa (including allotype V158 and F158) and FcγRIIIb (including allotype FcγRIIIb-NA1 and FcγRIIIb-NA2). Impairment or prevention of binding to FcRs by the spacer domain as disclosed herein prevents FcR-expressing cells from recognizing and destroying, or unintentionally activating the CAR-expressing cells, thereby minimizing or preventing immunological rejection and clearance of the therapeutically active cells. Whether a CAR exhibits binding activity to an FcR, such as to FcγRI or FcγRIIb as non-limiting examples, can be measured by methods known to those skilled in the art including FACS, ELISA, ALPHA screen (amplified luminescent proximity homogeneous assay) or BIACORE.

The spacer may comprise a detectable tag allowing the detection and/or the purification of the extracellular domain of the CAR, the CAR itself, and/or a cell expressing the CAR, e.g. a lymphocyte or host cell recombinantly expressing the CAR as described herein. Any tag allowing detection and/or purification is suitable such as known in the art or described herein. In a preferred embodiment of the present invention, the CAR comprises a spacer having a myc epitope tag, e.g. a c-myc epitope tag. Methods for tag detection are known in the art and include detection via flow cytometry or microscopy using antibodies specific for the tag, e.g. antibodies against c-myc or a portion thereof.

Suitable methods for purification of the CAR of the invention or of a lymphocyte recombinantly expressing the CAR as described herein are known in the art. Such methods for purification include preparative chromatographic separations and immunological separations based on antigen recognition/binding (e.g., recognition or binding to CSF1R) and/or based on the tag regions, e.g. comprising the use of antibodies specific for c-myc or a portion thereof.

As explained herein, in embodiments of the CAR comprising a spacer, it may be derived from any extracellular part of a protein having an extracellular domain, and is preferably derived from a biologically neutral portion of such extracellular domain. It is preferred that the spacer comprises the hinge domain of such extracellular domains, e.g. as provided among others by the CD nomenclature. Such are well known in the art and include, the hinge domain of CD8 and CD28. It is preferred that the hinge domain is preferably that of CD8. Most preferred is that the hinge domain is that of human CD8, for example having the amino acid sequence as shown herein in SEQ ID NO:7 (e.g. which may be encoded, for example, by SEQ ID NO:8).

In the context of the present invention, the extracellular domain/antigen binding region of the CAR as described herein comprises a moiety that provides specificity for CSF1R, and may be advantageously derived an antibody antigen biding domain as is known in the art, e.g. in preferred embodiments, an scFv. The extracellular domain/antigen binding region can be derived from, e.g. antibodies from different species as the lymphocyte donor or lymphocyte recipient, and may be chimeric or humanized, as long as the original binding activity to the target antigen is retained. Furthermore, it is considered that the extracellular domain of the CAR as described herein does not comprise an antibody Fc domain or a part thereof, including one or more of a CH1, CH2, or CH3 domains, as such elements increase the risk of adverse side reactions such as FcyR binding on administration to a subject.

In the context of the present invention, the term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g. hydroxyproline, γ-carboxyglutamate, and O-phosphoserine. Amino acid analogs refer to compounds that have the same basic chemical structure as a naturally occurring amino acid, i.e., an α-carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid but that function in a manner similar to a naturally occurring amino acid.

The CAR provided herein may exemplarily comprise or consist of the amino acid sequence of SEQ ID NO:23 or SEQ ID NO:24. Alternatively, the CAR provided herein exemplarily comprises or consists of a functional variant of SEQ ID NO:23 or SEQ ID NO:24. The term “functional variant” of a particular amino acid sequence encompasses variant amino acid sequences and/or fragments of the particular amino acid sequence or of the variant amino acid sequence, provided that the functional variant polypeptide exhibits or imparts the same functional activity as the particular amino acid sequence polypeptide. The term “variant amino acid sequence” of a particular amino acid sequence, e.g. of SEQ ID NO:23 or SEQ ID NO:24, refers to a functional polypeptide variant thereof, that does not have an amino acid sequence identical to the particular amino acid sequence, e.g. SEQ ID NO:23 or SEQ ID NO:24, but which polypeptide exhibits or imparts the same functional activity, in particular, specifically binding to CSF1R and exhibiting T cell activating activity on binding to CSF1R, when expressed by the lymphocyte. The functional variant can be any variant amino acid sequence polypeptide having an amino acid sequence that is at least 85%, 86%, 87%, 88%, 89%, 90%, 91%, 92%, 93%, 94%, 95%, 96%, 97%, 98% or 99% identical to the particular amino acid sequence, e.g. SEQ ID NO:23 or SEQ ID NO:24, provided that the variant sequence is characterized by the same functional activity as the original amino acid sequence. The term “fragment” of a particular amino acid sequence, e.g. SEQ ID NO:23 or SEQ ID NO:24 or its variant amino acid sequence, refers to a functional polypeptide variant thereof that does not have an amino acid sequence identical to the particular amino acid sequence, e.g. SEQ ID NO:23 or SEQ ID NO:24, but which polypeptide exhibits or imparts the same functional activity, e.g. specifically binding to CSF1R.

The term “at least X % identical to” in connection with the amino acid sequences/polypeptides and/or the nucleic acid sequences/nucleic acid molecules/polynucleotides as used herein describes the number of matches (“hits”) of identical amino acid or nucleic acid residues of two or more aligned sequences as compared to the number of residues making up the overall length of the compared sequences (or the overall compared portions thereof). In other terms, using an alignment, for two or more sequences or subsequences, the percentage of residues that are the same (e.g., at least 84%, 85%, 86%, 87%, 88%, 89%, 90%, 91%, 92%, 93%, 94%, 95%, 96%, 97%, 98% or 99% identity) may be determined when the (sub)sequences are compared and aligned for maximum correspondence over a window of comparison, or over a designated region as measured using a sequence comparison algorithm as known in the art, or when manually aligned and visually inspected.

Examples of algorithms for use in determining sequence identity include, for example, those based on CLUSTALW computer program (Thompson, Nucl. Acids Res. 2(1994), 4673-4680) or FASTA (Pearson and Lipman, Proc. Natl. Acad. Sci., 85(1988), 2444). Although the FASTA algorithm typically does not consider internal non-matching deletions or additions in sequences, i.e., gaps, in its calculation, this can be corrected manually to avoid an overestimation of the % sequence identity. CLUSTALW, however, does take sequence gaps into account in its identity calculations. Also available are the BLAST and BLAST 2.0 algorithms (Altschul, Nucl. Acids Res., 25(1977), 3389). The BLASTN program for nucleic acid sequences uses as default a word length (W) of 11, an expectation (E) of 10, M=5, N=4, and a comparison of both strands. For amino acid sequences, the BLASTP program uses as default a word length (W) of 3, and an expectation (E) of 10. The BLOSUM62 scoring matrix (Henikoff, Proc. Natl. Acad. Sci., 89(1989), 10915) uses alignments (B) of 50, expectation (E) of 10, M=5, N=4, and a comparison of both strands. Preferably the BLAST program is used in methods disclosed herein.

As described above, the herein provided CAR, e.g. the CAR recombinantly expressed by the lymphocyte provided herein, comprises a transmembrane domain. Any transmembrane portion of a protein e.g. of a signal transmitting receptor can be used in the construction of the CAR. Nonlimiting examples of proteins from which the transmembrane domain can be derived or taken include, but are not limited to, CD4, CD8 and CD28. In the present invention, it is preferred that the transmembrane domain comprises or consists of a CD28 transmembrane domain. Such a CD28 transmembrane domain may have an amino acid sequence of human or non-human origin, e.g. comprising or consisting of the amino acid sequence of the murine CD28 transmembrane domain (e.g. SEQ ID NO19) or comprising or consisting of the amino acid sequence of the human CD28 transmembrane domain (e.g. SEQ ID NO:21) as disclosed herein. It is most preferred that the transmembrane domain used in the CAR as disclosed herein comprise or consist of the transmembrane domain of human CD28 (SEQ ID NO:21, which may be encoded, for example, by SEQ ID NO:22).

The CAR of the invention also comprises an intracellular T cell activating domain. Such domains and their use in CAR construction, in particular, in the context of ACT are well known in the art. For example, such intracellular domains may comprise one or more stimulatory domains that transduce the signals necessary for lymphocyte (e.g. T cell) activation. Such intracellular signaling domains can include, for example, but not limited to, the intracellular signaling domain of CD3ζ, CD27, CD28, 4-1BB, OX40, ICOS and combinations thereof. Further, it may comprise an IL-2Rβ domain and a STAT3-binding motif such as YXXQ. It is preferred that the intracellular T cell activating domain of the CAR as described herein, or of the CAR expressed by the lymphocyte of the invention, e.g. for the use of the invention, comprises the signaling domain of the CD3ζ chain and/or at least one costimulatory domain that is an intracellular domain of an endogenous T cell receptor. Such costimulatory domain can be the intracellular domain of CD28 and/or CD137(4-1BB). The intracellular T cell activating domain of the CAR as described herein or the CAR expressed by the lymphocyte of the invention (e.g. for the use of the invention) preferably comprises the signaling domain of the CD3ζ chain and a costimulatory domain which comprises an intracellular domain of at least CD28 and/or CD137(4-1BB). The activity of the stimulatory signalling region(s), which provide(s) T cell activation, may be measured by the same means as determining T cell activation.

The invention further relates to polynucleotides encoding the CAR of the invention and to vectors comprising such a polynucleotide encoding the CAR of the invention. As a lymphocyte disclosed herein does not express the CAR as described herein endogenously, it is understood that such a lymphocyte has been genetically engineered so as to comprise the CAR.

The term “nucleic acid sequences” in accordance with the CAR, the genetically engineered lymphocyte and the methods as disclosed herein, relate to sequences of polynucleotides/nucleic acid molecules comprising purine- and pyrimidine bases. Thus, in the context of the invention, the terms “nucleic acid molecule” and “polynucleotide” may be interchangeably used and include DNA, such as cDNA, genomic DNA or synthetic forms of DNA, as well as RNA and mixed polymers comprising two or more of these molecules. It is understood that the term “RNA” as used herein comprises all forms of RNA including mRNA, tRNA and rRNA but also genomic RNA, such as in case of RNA of RNA viruses. Preferably, embodiments reciting “RNA” are directed to mRNA. The nucleic acid molecules/nucleic acid sequences of the invention may be of natural as well as of synthetic or semi-synthetic origin. Thus, the nucleic acid molecules may, for example, be nucleic acid molecules that have been synthesized according to conventional protocols of organic chemistry. The person skilled in the art is familiar with the preparation and the use of such nucleic acid molecules (see, e.g., Sambrook and Russel “Molecular Cloning, A Laboratory Manual”, Cold Spring Harbor Laboratory, N.Y. (2001)). Accordingly, further included are nucleic acid mimicking molecules known in the art such as synthetic or semi-synthetic derivatives of DNA or RNA and mixed polymers, both sense and antisense strands. They may contain additional non-natural or derivatized nucleotide bases, as will be readily appreciated by those skilled in the art. Such nucleic acid mimicking molecules or nucleic acid derivatives according to the invention include peptide nucleic acid (PNA), phosphorothioate nucleic acid, phosphoramidate nucleic acid, 2′-O-methoxyethyl ribonucleic acid, morpholino nucleic acid, hexitol nucleic acid (HNA) and locked nucleic acid (LNA), an RNA derivative in which the ribose ring is constrained by a methylene linkage between the 2′-oxygen and the 4′-carbon (see, for example, Braasch and Corey, Chemistry & Biology 8(2001), 1-7). PNA is a synthetic DNA-mimic with an amide backbone in place of the sugar-phosphate backbone of DNA or RNA, as described in, e.g., Nielsen et al., Science 254(1991), 1497; Egholm et al., Nature 365(1993), 666. Furthermore, it is envisaged for further purposes that nucleic acid molecules may contain, for example, thioester bonds and/or nucleotide analogues. Said modifications may be useful for the stabilization of the nucleic acid molecule against endo- and/or exonucleases in the genetically engineered cell. In a non-limiting example, the nucleic acid molecules/sequences disclosed herein may be transcribed by an appropriate vector containing a chimeric gene, which allows for the transcription of said nucleic acid molecule/sequence in the genetically engineered cell. In this respect, it is also to be understood that such polynucleotide can be used for “gene targeting” or “gene therapeutic” approaches. In another embodiment said nucleic acid molecules/sequences are labeled. Methods for the detection of nucleic acids are well known in the art, e.g., by Southern and Northern blotting, PCR or primer extension. Such embodiments may be useful for screening methods for verifying successful introduction of the nucleic acid molecules/sequences described above during gene therapy approaches. Said nucleic acid molecules/sequence(s) may be a recombinantly produced chimeric nucleic acid sequence comprising any of the aforementioned nucleic acid sequences either alone or in combination.

It is understood that the term comprising, as used above and throughout this description, denotes that further sequences, components and/or steps (e.g., when describing a method) can be included in addition to the specifically recited sequences, components and/or steps. However, this term also encompasses that the described subject-matter consists of exactly the recited sequences, components and/or method steps.

The genetically engineered lymphocyte of the invention may transiently or stably express the CAR as described herein. Additionally, the expression can be constitutive or constitutional, depending on the system used as known in the art. The polynucleotide or the vector encoding the polypeptide, may or may not be stably integrated into the cell's genome. Methods for achieving stable integration of introduced nucleic acids encoding desired proteins are well known in the art, and the invention encompasses the use of such methods as well as those described herein. Preferably, the herein provided lymphocyte (most preferably a primary human T cell) or the herein provided host cell which is preferably a lymphocyte has been genetically modified by introducing the polynucleotide or the vector comprising the polynucleotide into the lymphocyte.

As already stated above, the invention encompasses vectors comprising the polynucleotide encoding the CAR as described herein. As used herein, the term “vector” relates to a circular or linear nucleic acid molecule that can autonomously replicate in a host into which it has been introduced. The vector as used herein particularly refers to a plasmid, a cosmid, a virus, a bacteriophage and other vectors commonly used in genetic engineering as described herein or as is known in the art. Preferably, the disclosed vectors are suitable for the transformation of lymphocytes, preferably human lymphocytes and more preferably human primary lymphocytes, including but not limited to NK cells and T cells such as CD8+ T cells, CD4+ T cells, CD3+ T cells, γδ T cells, invariant T cells and NK T cells. Vectors in connection with the present invention comprise a nucleic acid sequence, e.g. the polynucleotide as described herein, encoding the CAR of the invention. As such, the vectors of use in connection with the present invention may encode the amino acid sequence SEQ ID NO:23 or SEQ ID NO:24, or a functional variant thereof, provided that the variant is characterized by specifically binding to CSF1R. It is understood that the vectors of use in connection with the present invention may also encode polypeptides comprising signaling domains to allow the proper processing and localization of the encoded polypeptide; accordingly, such vectors may encode CARs comprising membrane localization signaling peptides, e.g. as in SEQ ID NO:25 and SEQ ID NO:27.

It will be appreciated that the vectors disclosed herein may contain additional sequences to allow function such as replication or expression of a desired sequence in the cell system. For example, the vectors may comprise the polynucleotide encoding the CAR as described herein, under the control of regulatory sequences. The term “regulatory sequence” refers to DNA sequences that are necessary to affect the expression of coding sequences to which they are operably linked. As is understood in the art, the nature of such control sequences differs depending upon the host organism. In prokaryotes, control sequences generally include promoters, ribosomal binding sites, and terminators. In eukaryotes control sequences generally include promoters, terminators and, in some instances, enhancers, transactivators and/or transcription factors. The term “control sequence” is intended to include, at a minimum, all components the presence of which are necessary for expression, and may also include additional advantageous components, e.g., to allow replication. Regulatory or control sequences (including but not limited to promoters, transcriptional enhancers and/or sequences), which allow for induced or constitutive expression of the CAR as described herein, may be employed. Suitable promoters include but are not limited to the CMV promoter, the UBC promoter, PGK, the EF1A promoter, the CAGG promoter, the SV40 promoter, the COPIA promoter, the ACT5C promoter, or the TRE promoter (e.g., as disclosed in Qin et al., PLoS One. 5(2010), e10611); the Oct3/4 promoter (e.g., as disclosed in Chang et al., Molecular Therapy 9(2004), S367-S367 (doi: 10.1016/j.ymthe.2004.06.904)); or the Nanog promoter (e.g., as disclosed in Wu et al., Cell Res. 15(2005), 317-24).

The vectors of use in the present invention are preferably expression vectors. Suitable expression vectors have been widely described in the literature and the determination of the appropriate expression vector can be readily made by the skilled person using routine methods. Preferably, the vectors disclosed herein comprises a recombinant polynucleotide (i.e., a nucleic acid sequence encoding the CAR as described herein) as well as expression control sequences operably linked to the nucleotide sequence to be expressed. The vectors as provided herein preferably further comprise a promoter. The herein described vectors may also comprise a selection marker gene and a replication-origin ensuring replication in the host (i.e. a genetically engineered (e.g., transduced) lymphocyte such as a T cell). Moreover, the herein provided vectors may also comprise a termination signal for transcription. Between the promoter and the termination signal may be at least one restriction site or a polylinker to enable the insertion of a nucleic acid molecule encoding a polypeptide desired to be expressed (e.g. a polynucleotide encoding the CAR as disclosed herein). The use of expression vectors, including insertion of the encoding nucleic acid molecule/sequence and the harvest of the expressed polypeptide, is routine in the art. Non-limiting examples of vectors suitable for use in the present invention include cosmids, plasmids (e.g. naked or contained in liposomes) and viruses (e.g. retroviruses) that incorporate the nucleic acid molecules encoding the CAR. Of preferred use is a viral expression vector.

Methods for genetically engineering cells (in particular lymphocytes such as T cells and NK cells) to express polypeptides of interest are known in the art and can generally be divided into physical, chemical and biological methods. The appropriate method for given cell type and intended use can readily be determined by the skilled person using common general knowledge. Such methods for genetically engineering cells by introduction of nucleic acid molecules/sequences encoding the polypeptide of interest include but are not limited to chemical- and electroporation methods, calcium phosphate methods, cationic lipid methods, and liposome methods. The nucleic acid molecule/sequence to be transduced can be conventionally and highly efficiently transduced by using a commercially available transfection reagent and/or by any suitable method known in the art or described herein. In addition to methods of genetically engineering cells with nucleic acid molecules comprising or consisting of DNA sequences, the methods disclosed herein can also be performed with mRNA transfection. “mRNA transfection” refers to a method well known to those skilled in the art to transiently express a protein of interest, in the present case the CAR as described herein, in a lymphocyte, e.g., a T cell. Accordingly, the methods herein may be used to genetically engineer a lymphocyte to transiently or stably (either constitutively or conditionally) express the polypeptide of interest. For example, with respect to mRNA transfection, lymphocytes may be electroporated with the mRNA coding for the CAR as described herein by using an electroporation system (such as e.g. Gene Pulser, Bio-Rad) and thereafter cultured by standard cell culture protocols (see, e.g., Zhao et al., Mol Ther. 13(2006), 151-159).

Physical methods for introducing a polynucleotide into a host cell include calcium phosphate precipitation, lipofection, particle bombardment, microinjection, electroporation, and the like; see, e.g., Sambrook et al., 2012, Molecular Cloning: A Laboratory Manual, volumes 1-4, Cold Spring Harbor Press, NY. Biological methods for introducing a polynucleotide of interest into a host cell include the use of DNA and RNA vectors. Viral vectors, and especially retroviral vectors, have become the most widely used method for inserting genes into mammalian (e.g., human cells such as a T cells). Accordingly, although retroviral vectors are preferred for use in the methods and cells disclosed herein, viral vectors can be derived from a variety of different viruses, including but not limited to lentivirus, poxviruses, herpes simplex virus I, adenoviruses and adeno-associated viruses; see, e.g. U.S. Pat. Nos. 5,350,674 and 5,585,362. Non-limiting examples of suitable retroviral vectors for transducing T cells include SAMEN CMV/SRa (Clay et al., J. Immunol. 163(1999), 507-513), LZRS-id3-IHRES (Heemskerk et al., J. Exp. Med. 186(1997), 1597-1602), FeLV (Neil et al., Nature 308(1984), 814-820), SAX (Kantoff et al., Proc. Natl. Acad. Sci. USA 83(1986), 6563-6567), pDOL (Desiderio, J. Exp. Med. 167(1988), 372-388), N2 (Kasid et al., Proc. Natl. Acad. Sci. USA 87(1990), 473-477), LNL6 (Tiberghien et al., Blood 84(1994), 1333-1341), pZipNEO (Chen et al., J. Immunol. 153(1994), 3630-3638), LASN (Mullen et al., Hum. Gene Ther. 7(1996), 1123-1129), pG1XsNa (Taylor et al., J. Exp. Med. 184(1996), 2031-2036), LCNX (Sun et al., Hum. Gene Ther. 8(1997), 1041-1048), SFG (Gallardo et al., Blood 90(1997), LXSN (Sun et al., Hum. Gene Ther. 8(1997), 1041-1048), SFG (Gallardo et al., Blood 90(1997), 952-957), HMB-Hb-Hu (Vieillard et al., Proc. Natl. Acad. Sci. USA 94(1997), 11595-11600), pMV7 (Cochlovius et al., Cancer Immunol. Immunother. 46(1998), 61-66), pSTITCH (Weitjens et al., Gene Ther 5(1998), 1195-1203), pLZR (Yang et al., Hum. Gene Ther. 10(1999), 123-132), pBAG (Wu et al., Hum. Gene Ther. 10(1999), 977-982), rKat.43.267bn (Gilham et al., J. Immunother. 25(2002), 139-151), pLGSN (Engels et al., Hum. Gene Ther. 14(2003), 1155-1168), pMP71 (Engels et al., Hum. Gene Ther. 14(2003), 1155-1168), pGCSAM (Morgan et al., J. Immunol. 171(2003), 3287-3295), pMSGV (Zhao et al., J. Immunol. 174(2005), 4415-4423), or pMX (de Witte et al., J. Immunol. 181(2008), 5128-5136). Most preferred are lentiviral vectors. Non-limiting examples of suitable lentiviral vectors for transducing T cells are, e.g. PL-SIN lentiviral vector (Hotta et al., Nat Methods. 6(2009), 370-376), p156RRL-sinPPT-CMV-GFP-PRE/NheI (Campeau et al., PLoS One 4(2009), e6529), pCMVR8.74 (Addgene Catalogoue No.: 22036), FUGW (Lois et al., Science 295(2002), 868-872, pLVX-EF1 (Addgene Catalogue No.: 64368), pLVE (Brunger et al., Proc Natl Acad Sci USA 111(2014), E798-806), pCDH1-MCS1-EF1 (Hu et al., Mol Cancer Res. 7(2009), 1756-1770), pSLIK (Wang et al., Nat Cell Biol. 16(2014), 345-356), pLJMI (Solomon et al., Nat Genet. 45(2013), 1428-30), pLX302 (Kang et al., Sci Signal. 6(2013), rs13), pHR-IG (Xie et al., J Cereb Blood Flow Metab. 33(2013), 1875-85), pRRLSIN (Addgene Catalogoue No.: 62053), pLS (Miyoshi et al., J Virol. 72(1998), 8150-8157), pLL3.7 (Lazebnik et al., J Biol Chem. 283(2008), 11078-82), FRIG (Raissi et al., Mol Cell Neurosci. 57(2013), 23-32), pWPT (Ritz-Laser et al., Diabetologia. 46(2003), 810-821), pBOB (Marr et al., J Mol Neurosci. 22(2004), 5-11), and pLEX (Addgene Catalogue No.: 27976).

Chemical means for introducing a polynucleotide into a host cell include colloidal dispersion systems, such as macromolecule complexes, nanocapsules, microspheres, beads, and lipid-based systems including oil-in-water emulsions, micelles, mixed micelles, and liposomes. An exemplary colloidal system for use as a delivery vehicle in vitro and in vivo is a liposome (e.g., an artificial membrane vesicle). Other methods of state-of-the-art targeted delivery of nucleic acids are available, such as delivery of polynucleotides with targeted nanoparticles or other suitable sub-micron sized delivery system. In the case where a non-viral delivery system is utilized, an exemplary delivery vehicle is a liposome. The use of lipid formulations is contemplated for the introduction of the nucleic acids into a host cell (in vitro, ex vivo or in vivo). Alternatively, the nucleic acid may be associated with a lipid. The nucleic acid associated with a lipid may be encapsulated in the aqueous interior of a liposome, interspersed within the lipid bilayer of a liposome, attached to a liposome via a linking molecule that is associated with both the liposome and the oligonucleotide, entrapped in a liposome, complexed with a liposome, dispersed in a solution containing a lipid, mixed with a lipid, combined with a lipid, contained as a suspension in a lipid, contained or complexed with a micelle, or otherwise associated with a lipid. Lipid, lipid/DNA or lipid/expression vector associated compositions are not limited to any particular structure in solution. For example, they may be present in a bilayer structure, as micelles, or with a “collapsed” structure. They may also simply be interspersed in a solution, possibly forming aggregates that are not uniform in size or shape. Lipids may be naturally occurring or synthetic lipids. Lipids suitable for use in methods of nucleic acid molecule delivery to a host cell (i.e., to genetically engineer the host cell) can be obtained from commercial sources. For example, dimyristyl phosphatidylcholine (“DMPC”) can be obtained from Sigma, St. Louis, Mo.; dicetyl phosphate (“DCP”) can be obtained from K & K Laboratories (Plainview, N.Y.); cholesterol (“Choi”) can be obtained from Calbiochem-Behring; dimyristyl phosphatidylglycerol (“DMPG”) and other lipids may be obtained from Avanti Polar Lipids, Inc. (Birmingham, Ala.).

Regardless of the method used to introduce the polynucleotide or vector into a host cell, in order to confirm the presence of the recombinant DNA sequence (i.e., inside the lymphocyte of the invention or to confirm that the target cell has been genetically engineered according to the methods disclosed herein), a variety of assays may be performed. Such assays include, for example, “molecular biological” assays well known to those of skill in the art such as Southern and Northern blotting, RT-PCR and PCR, “biochemical” assays such as detecting the presence or absence of a particular polypeptide, e.g. by immunological means (ELISAs and/or Western blots) or by assays described herein to identify whether the cell exhibits a property or activity associated with the engineered polypeptide, é.g. assays to assess whether the lymphocyte exhibits a desired activity such as the specific binding to CSF1R.

The genetic engineering methods disclosed herein are applied to lymphocytes, preferably T cells. As known in the art, T cells are cells of the adaptive immune system that recognize their target in an antigen specific manner. These cells are characterized by surface expression of CD3 and a T cell receptor (TCR), which recognizes a cognate antigen in the context of a major histocompatibility complex (MHC). T cells may be further subdivided in CD4+ or CD8+ T cells. CD4+ T cells recognize an antigen through their TCR in the context of MHC class II molecules that are predominantly expressed by antigen-presenting cells. CD8+ T cells recognize their antigen in the context of MHC class I molecules that are present on most cells of the human body. Methods for identifying, separating and maintaining specific sub-populations of T cells (e.g., as a culture of primary T cells) such as CD3+, CD4+ and/or CD8+ T cells from a cell population (such as a population of peripheral blood mononuclear cells e.g., having been isolated from a patient for the purpose of autologous cell therapy) are well known to those skilled in the art and include flow cytometry, microscopy, immunohistochemistry, RT-PCR or western blot (Kobold, J Natl Cancer Inst 107(2015), 107).

As described herein, the genetically engineered lymphocyte of the present invention is recombinantly modified with a nucleic acid sequence/polynucleotide encoding (and driving/permitting expression of) the herein described CAR. In the case of cells bearing natural anti-tumor specificity (such as tumor-infiltrating lymphocytes (TIL see, e.g., Dudley et al., J Clin Oncol. 31(2013), 2152-2159)) or antigen-specific cells sorted from the peripheral blood of patients for their tumor-specificity by flow cytometry (Hunsucker et al., Cancer Immunol Res. 3(2015), 228-235), the genetically engineered cells described herein may only be modified to express the CAR. However, the genetically engineered T cell of the invention may be further engineered with additional nucleic acid molecules to express, in addition to the exogenous CAR as described herein, other polypeptides of use in ACT, e.g., with a nucleic acid sequence encoding a further, exogenous, T cell receptor or a further chimeric antigen receptor (CAR) specific for a tumor of interest. Alternately or additionally, the T cell can be further genetically modified to disrupt the expression of the endogenous T cell receptor, such that it is not expressed or expressed at a reduced level as compared to a T cell absent of such modification.

In the present invention, it is preferred that both the lymphocyte or host cell for use in the methods of the invention are non-alloreactive. In case the non-alloreactive lymphocyte or host cell is a T cell, it is further preferred that such a T cell comprises genetic mutations to reduce or eliminate expression of the endogenous TCR, or of the endogenous TCR alpha or beta chain genes. In the context of the present invention, the term “endogenous” refers to molecules which are naturally not presented in and/or on the surface of a cell, e.g. a T cells, and which are not (endogenously) expressed in or on normal (non-transduced) cells, e.g. T cells. Accordingly, the term “exogenous” refers to molecules which do not naturally occur in or on cells, e.g. T cells and relates to molecules which are incorporated into the cell, e.g. a T cell, which are naturally not presented in and/or on the surface of the cell and which are not (endogenously) expressed in or on normal (non-transduced) cells. In the context of the present invention, these artificially introduced molecules are presented in and/or on the surface of cells, e.g. T cells, after genetic engineering as accomplished by methods known in the art or as disclosed herein. Further, as used herein, the term “reduced expression” and analogous terms refer to any reduction in the expression of the endogenous T cell receptor at the cell surface of a genetically modified cell when compared to a control cell. The term reduced can also refer to a reduction in the percentage of cells in a population of cells that express an endogenous polypeptide (i.e., an endogenous TCR) at the cell surface when compared to a population of control cells. Accordingly, the term “reduced expression” in connection with the expression of an endogenous T cell receptor relates to a partial knockdown, while the term “eliminated expression” relates to a complete, or essentially complete knockdown of the endogenous TCR within the population of genetically modified cells. In this context, in case the T cell comprises genetic mutations to reduce or eliminate expression of the endogenous TCR, or of the endogenous TCR alpha or beta chain genes as described herein.

In the present invention, the lymphocyte or host cell may further recombinantly express an exogenous cytokine receptor.

The lymphocyte or host cell expressing the CAR of the invention is of particular use in the treatment of cancer characterized by the expression of colony stimulating factor 1 receptor (CSF1R) and can successfully be employed in pharmaceutical compositions. In this context, it should be understood that the pharmaceutical composition may also comprise the lymphocytes as obtained by the methods disclosed herein.

The terms “treatment”, “treating” and the like are used herein to generally mean obtaining a desired pharmacological and/or physiological effect. The effect may be prophylactic in terms of completely or partially preventing a disease or symptom thereof, and/or may be therapeutic in terms of partially or completely curing the disease or condition, and/or adverse effect attributed to the disease or condition. The term “treatment” as used herein covers any treatment of a disease or condition in a subject and includes: (a) preventing and/or ameliorating a proliferative disease (preferably cancer) from occurring in a subject that may be predisposed to the disease; (b) inhibiting the disease, i.e. arresting its development, such as inhibition of cancer progression; (c) relieving the disease, i.e. causing regression of the disease, such as the repression of cancer; and/or (d) preventing, inhibiting or relieving any symptom or adverse effect associated with the disease or condition. Preferably, the term “treatment” as used herein relates to medical intervention of an already manifested disorder, e.g. the treatment of a diagnosed cancer, in particular characterized by the expression of CSF1R.

“Characterized by the expression of colony stimulating factor 1 receptor (CSF1R)” as used herein indicates that the cancerous or precancerous parenchyma when considered as a whole expresses CSF1R. Accordingly, a cancer or precancerous tissue is characterized by the expression of CSF1R not only where all or a portion of the cancerous or precancerous cells within the parenchyma themselves express CSF1R, but also wherein any cells within the diseased parenchyma express CSF1R. For example, a cancer or pre-cancer may also be characterized by the expression of CSF1R where the cancer or precancerous cells do not express CSF1R, but where immune cells resident within the diseased tissue express CSF1R (e.g. infiltrating lymphocytes, in particular tumor infiltrating lymphocytes (TIL)).

The term “pharmaceutical composition” can be used interchangeably with “medicament” and generally relates to a composition for administration to a patient, preferably a human patient. Furthermore, in the context of the present invention, such patient suffers from a disease characterized by the expression of CSF1R, wherein said disease is a malignant disease, especially a cancer of the blood. However, the composition of the invention as described herein may also be a composition for diagnosing further comprising, optionally, means and methods for detection. The pharmaceutical composition as disclosed herein may be administered locally or systematically. As such, the composition may be administered by any suitable way, including parenteral, transdermal, intraluminal, intraarterial, intrathecal administration and direct injection into the tissue or tumor, however, parenteral administrations is the preferred application method. Preparations for such parenteral administration include sterile aqueous or non-aqueous solutions, suspensions and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishes, electrolyte replenishers (such as those based on Ringer's dextrose) and the like. Preservatives and other additives may also be present such as, for example, antimicrobials, antioxidants, chelating agents, and inert gases and the like. In addition, the pharmaceutical composition of the present invention might comprise proteinaceous carriers, like, e.g., serum albumin or immunoglobulins, preferably of human origin and may also comprise, optionally, suitable formulations stabilizers and/or excipients.

The pharmaceutical composition/medicament of the present invention may further comprise a pharmaceutically acceptable carrier. Examples of suitable pharmaceutical carriers are well-known in the art and include phosphate buffered saline solutions, water, emulsions, such as oil/water emulsions, various types of wetting agents, sterile solutions or others. Compositions comprising such carriers can be formulated by well-known conventional methods. These pharmaceutical compositions can be administered to the subject at a suitable dose. The dosage regimen will be determined by the attending physician and clinical factors. As is well known in the medical arts, dosages for any one patient depend upon many factors, including the patient's size, body surface area, age, the particular compound to be administered, sex, time and route of administration, general health, and other drugs being administered concurrently.

It is envisaged that the pharmaceutical composition of the invention may comprise, in addition to the lymphocyte recombinantly expressing the CAR as described herein, further biologically active agents, depending on the intended use of the pharmaceutical composition. Such agents may include medicaments acting on the gastro-intestinal system, cytostatic drugs, drugs preventing hyperuricemia, drugs inhibiting immunoreactions (e.g. corticosteroids), drugs acting on the circulatory system and/or agents such as T cell co-stimulatory molecules or cytokines known in the art.

The pharmaceutical compositions described herein can be used in combination with a chemotherapeutic agent. Exemplary chemotherapeutic agents include an anthracycline (e.g., doxorubicin (e.g., liposomal doxorubicin)). a vinca alkaloid (e.g., vinblastine, vincristine, vindesine, vinorelbine), an alkylating agent (e.g., cyclophosphamide, decarbazine, melphalan, ifosfamide, temozolomide), an immune cell antibody (e.g., alemtuzamab, gemtuzumab, rituximab, ofatumumab, tositumomab, brentuximab), an antimetabolite (including, e.g., folic acid antagonists, pyrimidine analogs, purine analogs and adenosine deaminase inhibitors (e.g., fludarabine)), an mTOR inhibitor, a TNFR glucocorticoid induced TNFR related protein (GITR) agonist, a proteasome inhibitor (e.g., aclacinomycin A, gliotoxin or bortezomib), an immunomodulator such as thalidomide or a thalidomide derivative (e.g., lenalidomide). General chemotherapeutic agents considered for use in combination therapies also include but are not limited to anastrozole, bicalutamide, bleomycin sulfate, busulfan, capecitabine, N4-pentoxycarbonyl-5-deoxy-5-fluorocytidine, carboplatin, carmustine, chlorambucil, cisplatin, cladribine, cyclophosphamide, cytarabine, cytosine arabinoside, cytarabine liposome injection, dacarbazine, dactinomycin, daunorubicin hydrochloride, daunorubicin citrate liposome injection, dexamethasone, docetaxel, doxorubicin hydrochloride, etoposide, fludarabine phosphate, 5-fluorouracil, flutamide, tezacitibine, Gemcitabine, hydroxyurea (Hydrea®), Idarubicin, ifosfamide, irinotecan, L-asparaginase, leucovorin calcium, melphalan, 6-mercaptopurine, methotrexate, mitoxantrone, mylotarg, paclitaxel, Yttrium90/MX-DTPA, pentostatin, tamoxifen citrate, teniposide, 6-thioguanine, thiotepa, tirapazamine, topotecan hydrochloride, vinblastine, vincristine, and vinorelbine.

Anti-cancer agents of particular interest for combination with the genetically engineered lymphocyte based methods and compounds disclosed herein include: anthracyclines; alkylating agents; antimetabolites; drugs that inhibit either the calcium dependent phosphatase calcineurin or the p70S6 kinase FK506) or inhibit the p70S6 kinase; mTOR inhibitors; immunomodulators; anthracyclines; vinca alkaloids; proteosome inhibitors; GITR agonists; protein tyrosine phosphatase inhibitors; a CDK4 kinase inhibitor; a BTK inhibitor; a MKN kinase inhibitor; a DGK kinase inhibitor; or an oncolytic virus.

Exemplary antimetabolites include, without limitation, pyrimidine analogs, purine analogs and adenosine deaminase inhibitors): methotrexate, 5-fluorouracil, floxuridine, cytarabine, 6-mercaptopurine, 6-thioguanine, fludarabine phosphate, pentostatin, pemetrexed, raltitrexed, cladribine, clofarabine, azacitidine, decitabine and gemcitabine.

Exemplary alkylating agents include, without limitation, nitrogen mustards, uracil mustard, ethylenimine derivatives, alkyl sulfonates, nitrosoureas, triazenes, chlormethine, cyclophosphamide, ifosfamide, melphalan, chlorambucil, pipobroman, triethylenemelamine, triethylenethiophosphoramine, temozolomide, thiotepa, busulfan, carmustine, lomustine, streptozocin, dacarbazine, oxaliplatin, temozolomide, dactinomycin, melphalan, altretamine, carmustine, bendamustine, busulfan, carboplatin, lomustine, cisplatin, chlorambucil, cyclophosphamide, dacarbazine, altretamine, ifosfamide, prednumustine, procarbazine, mechlorethamine, streptozocin, thiotepa, cyclophosphamide, and bendamustine HCl.

The invention further envisages the co-administration protocols with other compounds, e.g. molecules capable of providing an activation signal for immune effector cells, for cell proliferation or for cell stimulation.

In the foregoing detailed description of the invention, a number of individual elements, characterizing features, techniques and/or steps are disclosed. It is readily recognized that each of these has benefit not only individually when considered or used alone, but also when considered and used in combination with one another. Accordingly, to avoid exceedingly repetitious and redundant passages, this description has refrained from reiterating every possible combination and permutation. Nevertheless, whether expressly recited or not, it is understood that such combinations are entirely within the scope of the presently disclosed subject matter.

All technical and scientific terms used herein, unless otherwise defined, are intended to have the same meaning as commonly understood by one of ordinary skill in the art. Reference to techniques employed herein are intended to refer to the techniques as commonly understood in the art, including variations on those techniques or substitutions of equivalent techniques that would be apparent to one of skill in the art.

### 5. EXAMPLES

### 5.1 Example 1: CSF1R Expression in Samples of AML

The following example demonstrates the identification of Colony Stimulating Factor 1 Receptor (CSF1R) as an acute myeloid leukemia (AML)-specific marker.

### 5.1.1 Search of Public Databases

As a first step, identification of potential AML-specific target structures was realized by using the public databases “Gene Expression Profiling Interactive Analysis” (GEPIA) and Bloodspot.eu. Both databases use bulk RNA Sequencing data from published patient cohorts. GEPIA was used to assess CSF1R expression pattern for different cancer entities compared to healthy tissue. CSF1R was identified to be highly upregulated in AML samples compared to healthy bone marrow control (FIG. 1A). This result was verified by using Bloodspot.eu which allowed evaluation of different published clinical cohorts. In line with the previous findings, an upregulation of CSF1R was observed for different AML subtypes in a large-scale datasets (Leukemia MILE study) (FIG. 1B).

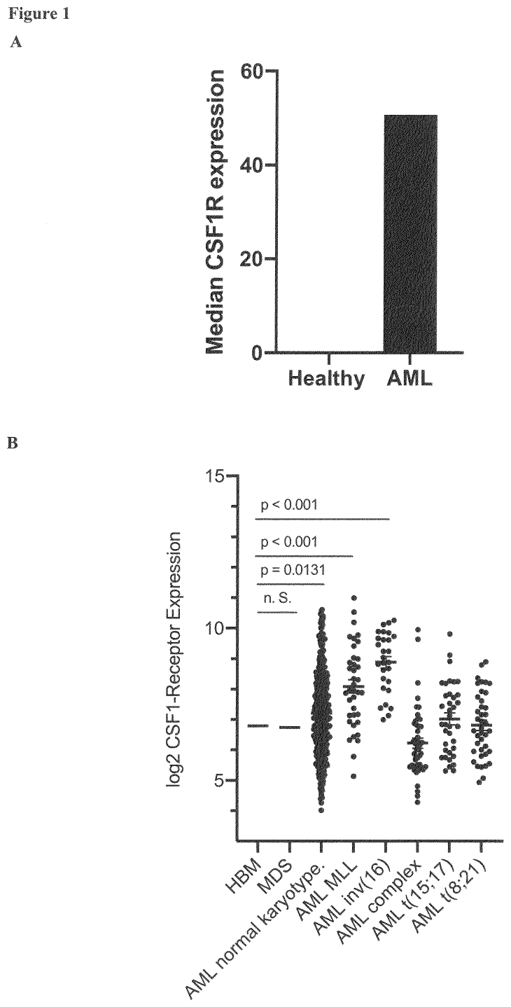

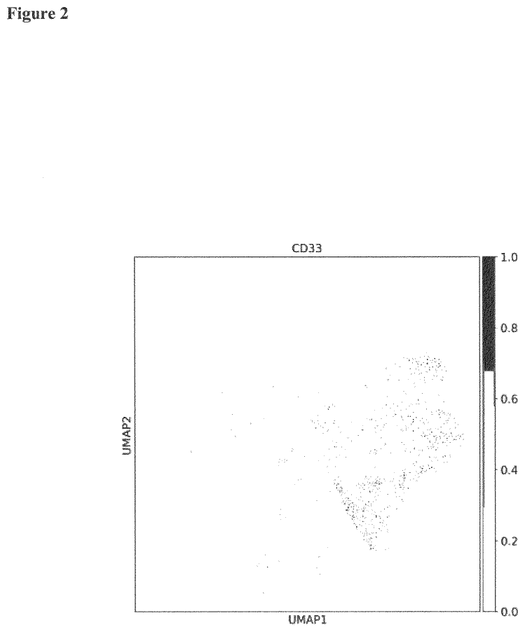

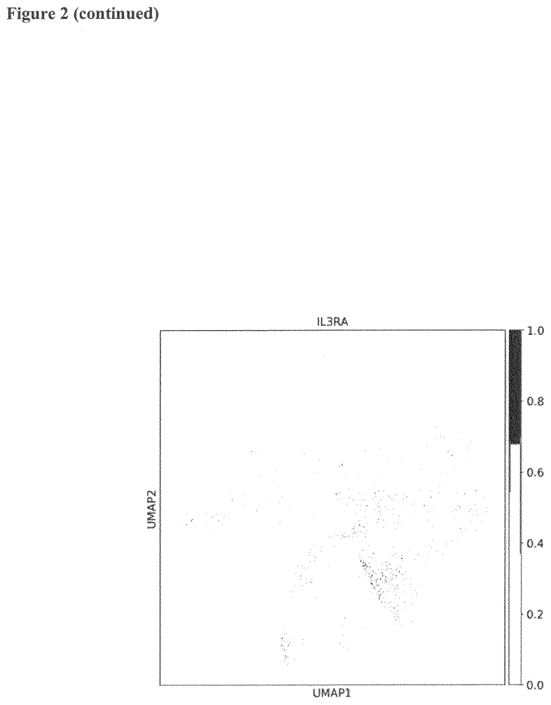

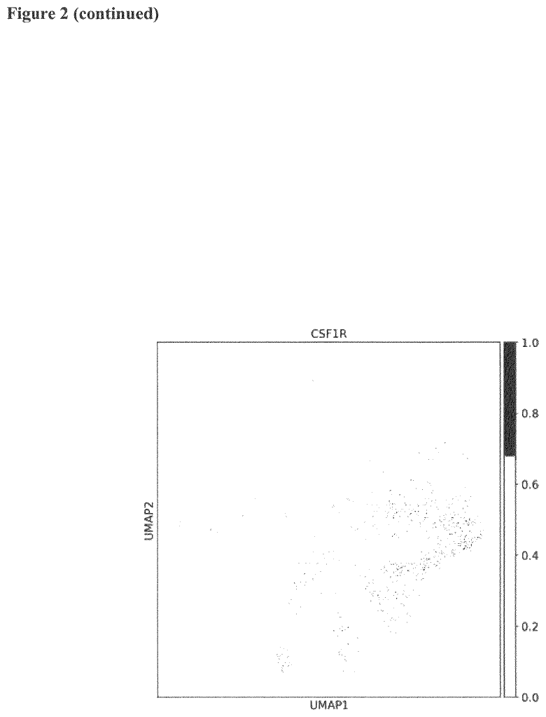

The prior art has recently shown anti-tumor efficacy of small molecule CSF1R inhibitors (Edwards et al., Blood (2019) 133 (6): 588-599). However, CSF1R expression has mostly been described on paracrine support cells and only to a lesser extent on AML blasts. To further examine these findings, single-cell RNA Sequencing (scRNA Seq) of a published AML dataset (Van Galen et al. Cell (2019); 176(6):1265-1281.e24) has been used. The analysis revealed broad expression of CSF1R on malignant AML cells of different molecular AML subtypes, very similar to common AML-associated antigens such as CD33, and CD123 (IL3RA) (FIG. 2). Importantly, in contrast to the prior art publication, it was possible to clearly demonstrate expression of CSF1R on malignant AML blasts using scRNA Sequencing.

The analyses surprisingly revealed CSF1R as potential marker for AML.

### 5.1.2 Analysis of CSFIR Expression in Patient Samples of AML Blasts and in AML Cell Lines

To verify the results obtained from sequencing analysis which identified CSF1R as potential AML marker, CSF1R expression on myeloid blasts of human AML patients as well as on AML cell lines was determined using FACS analysis.

### 5.1.2.1 Cell Line Culture

Human AML cell lines PL-21, THP-1, MV4-11, OCI-AML3, MOLM-13, U937 and SU-DHL-4 were purchased from ATCC (USA). All cell lines were cultured in RPMI containing 20% FBS, 2 mM L-Glutamine, 100 U/ml penicillin and 100 μg/ml streptomycin. Cells were grown at 37° C. in a humidified incubator with 5% CO₂. Short tandem repeat (STR) profiling was used to verify their origins. Cells were regularly tested for mycoplasma contamination using polymerase chain reaction (PCR). Cultures were maintained by addition or replacement of the respective medium after the cells have been centrifuged for 5 min, at 400 g at room temperature. All cell lines were lentivirally transduced with a pCDH-EF1a-eFly-eGFP plasmid. After transduction, enhanced green-fluorescent protein (eGFP) positive cells were single cell sorted using a BD FACSAria™ III Cell Sorter and expression of firefly luciferase (fLuc) was verified using Bio-Glo™ Luciferase Assay System. Cells were frozen in medium containing 90% FCS and 10% DMSO and stored at −80° C. or in liquid nitrogen for long-term storage.

### 5.1.2.2 AML Blast Isolation and Culture

Primary AML blasts were obtained from the bone marrow (BM) or peripheral blood (PB) of patients suffering from acute myeloid leukemia (AML) after written informed consent in accordance with the Declaration of Helsinki and approval by the Institutional Review Board of the Ludwig-Maximilians Universitat (Munich, Germany). Bone marrow aspirates from said patients are enriched for AML blasts either through density centrifugation or lysis of red blood cells using osmotic gradient solutions and frozen in the liquid nitrogen as described. Prior to T cell-based assay, bone marrow aspirates are thawed and T cells are depleted using a CD3 positive selection kit (StemCell Technologies).

Primary AML samples were either cultured in IMDM basal medium supplemented with 15% BIT 9500 serum substitute and beta-Mercaptoethanol (104 M), 100 ng/ml SCF, 50 ng/ml FLT3-Ligand, 20 ng/ml G-CSF, 20 ng/ml IL-3, 1 μM UM729 and 500 nM SR1 as described in Pabst et al., Nature Methods (2014), 11: 436-442 for FACS analysis or alternatively in alpha-MEM-supplemented with 12.5% horse serum, 1% penicillin/streptomycin, 1% L-glutamine, G-CSF, IL-3, TPO and 2-Mercaptoethanol on irradiated MS-5 (murine bone marrow stromal cells) for co-culture experiments as described in Gosliga et al., Experimental Hematology (2007), 35(10):1538-1549.

### 5.1.2.3 FACS Analysis

Flow cytometric analysis was carried out using a BD LSRFortessa™ II. Flow cytometric data was analyzed using FlowJo V10.3 software. All staining steps were conducted on ice, as rapid internalization of the CSF1R-receptor has been demonstrated. Cells were centrifuged at 200-400 g for 5 min at 4° C. in a pre-cooled centrifuge. For staining of primary AML blasts and AML cell lines a maximum of 10⁶ cells were counted and transferred to a U bottom 96 well plate. Cells were washed twice with ice cold phosphate-based saline (PBS) containing 2% FBS. Cells were incubated for 15 min on ice with 5 μl of human TrueStain FcX™ (Biolegend, USA) to prevent unspecific binding of antibodies. CSF1R was stained on ice for 30 minutes in the dark using an anti-human CSF1R antibody conjugated to PerCP-Cy5.5 (Biolegend, Clone 9-4D2-1E4) or an unconjugated anti-human m-CSF-R/CD115 Antibody (R&D, Clone 61701), followed by secondary staining with Alexa Fluor© 647 rat anti-mouse IgG (H+L) antibody (Jackson ImmunoResearch, USA). Positive staining was validated using isotype controls (PerCP/Cy5.5 Rat IgG1, k, Biolegend, Clone: RTK2071; Mouse IgG1 Isotype Control, R&D Systems, Clone 11711). Dead cells were excluded after staining with a fixable viability dye (eFluor™ 780, eBioscience, USA).

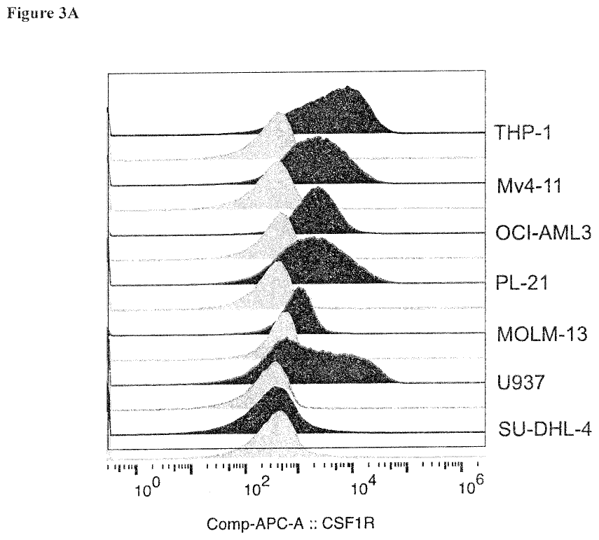

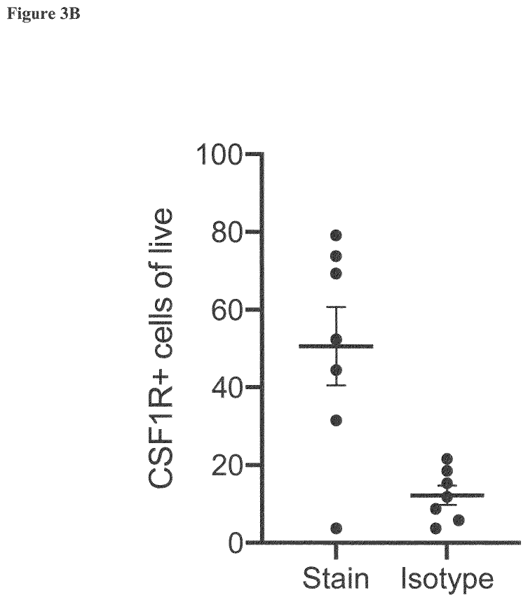

As shown in FIGS. 3A, staining revealed homogenous expression of CSF1R on AML cell lines THP-1, MV4-11, OCI-AML-3 and PL-21. To verify these results, two more AML cell lines (MOLM-13, U937) were stained for CSF1R, which showed positive staining as well (FIG. 3A). SU-DHL-4 cells, a Non-Hodgkin B cell lymphoma cell line with published negativity for CSF1R (Lamprecht et al., Nat Med. (2010), 16(5):571-9) was used as a negative control. In summary, relevant expression of CSF1R across six different AML cell lines has been demonstrated. Next, expression of CSF1R on primary human AML blasts was verified. Frozen bone marrow (BM) samples of AML patients were thawed, cultured for 24 hours in a cytokine rich medium as described in Example 5.1.2.2 and stained for CSF1R expression. Gating for AML blasts was carried out by using the conventional SSC-CD45 gating strategy. As shown in FIG. 3B, staining of the cultured primary AML blasts revealed high expression of CSF1R.

### 5.2 Example 2: In Vitro Studies of CSF1R as a Therapeutic Target

The following example demonstrates CSF1R as targeting antigen for therapy, e.g. by modified T cells.

### 5.2.1 Design of CSF1R Specific Chimeric Antigen Receptors (CAR)

Example 1 revealed that AML blasts could readily be identified based on their CSF1R expression. To assess whether CSF1R could also serve as a target for anti-tumor therapy, a second-generation CAR T cell was developed to specifically recognize CSF1R. The construct was designed as follows: human CD8alpha signal peptide—anti-CSF1R VH—(G₄S)₄-Linker-anti-CSF1R VL—myc-tag—murine CD8 hinge—murine CD28 transmembrane domain—murine CD28 intracellular domain—murine CD3zeta domain.

The exemplary CAR used in the present examples has the amino acid sequence of SEQ ID NO:37 as encoded by the nucleic acid of SEQ ID NO:38. More specifically, the experimentally tested CAR comprises humanized scFv of SEQ ID NO:1 and comprises murine sequences of the CD8 hinge (SEQ ID NO:5), the CD28 transmembrane domain (SEQ ID NO:19), the CD28 co-stimulatory domain (SEQ ID NO:31), and the T cell activating domain of CD3zeta (SEQ ID NO:29). As demonstrated herein the CAR comprising the murine sequences was fully functional in human cells. The functionality of murine sequences in human cells is recognized in the art, but is considered as less optimal than the corresponding human sequences. Accordingly, a fully human CAR construct, e.g. having the amino acid sequence of SEQ ID NO:25 or SEQ ID NO:27, will necessarily exhibit at least the same or improved activity leading to comparable or improved results.

The anti-CSF1R single chain variable fragment (scFv) was designed based on the sequence of the heavy and light chain variable domains of the anti-CSF1R antibody clone 2F11-e7 reported in EP-B1 2 510 010. A myc tag was included to readily detect CAR expression. The CD19 CAR is constructed in a similar fashion as the anti-CSF1R CAR. Anti-CD19 CAR T cells were designed based on the anti-CD19-CAR-FMC63-28Z CAR T cells disclosed in WO 2015/187528.

To compare efficacy of anti-CSF1R CAR T cells to established therapies, to the cells were compared to anti-CD33 CAR T cells. Thus, second generation anti-CD33 CAR T cells were generated, harboring the same functional domains as the anti-CSF1R CAR T cells. More precisely, design of the anti-CD33 CAR T cells were as follows: CD8alpha signal peptide—anti-CD33scFv—c-myc tag—CD8 hinge—CD28 transmembrane—CD28 intracellular domain—human CD3zeta domain. The anti-CD33 scFv was designed based on the anti-CD33 antibody gemtuzumab reported in U.S. Pat. No. 5,773,001.

T cells were isolated, cultured and transduced with either anti-CSF1R-CAR or anti-CD33-CAR as described in Example 5.2.3.

### 5.2.2 Virus Production

For virus production, retroviral pMP71 (Schambach et al., Mol Ther. (2000); 2(5):435-45) vectors carrying the sequence of the relevant receptor were stably expressed in packaging cell lines 293Vec-Galv and 293Vec-RD114 by routine methods known in the art. Producer cell lines 293Vec-RD114-CAR-CSF1R, 293Vec-RD114-CAR-CD19 and 293Vec-RD114-CAR-CD33 were established.

### 5.2.3 T Cell Culture and T Cell Transduction

For T cell transduction, human peripheral blood mononuclear cells (PBMC) were isolated from healthy donors using density gradient centrifugation. After isolation of the PBMC fraction, cells were washed twice with PBS. Subsequently, T cells were isolated using anti-CD3 microbeads (Miltenyi Biotec, Germany). Isolated T cells were counted, adjusted to a cell concentration of 10⁶/ml and stimulated for 48 hours using Human T-Activator CD3/CD28 Dynabeads® (Life Technologies, Darmstadt, Germany) in complete human T cell medium containing 2.5% human Serum, 2 mM L-Glutamine, 100 U/ml penicillin, 100 μg/ml streptomycin, 1% non-essential amino acids, 1% sodium pyruvate and supplemented with recombinant human IL-2 (Peprotech, Hamburg, Germany) and IL-15 (Peprotech, Hamburg, Germany). T cell transduction was carried out by retroviral transduction. Retroviral particles were generated from producer cell lines stably expressing the desired constructs, as previously described (Example 5.2.2). Virus supernatant was added to retronektin-coated 24 well plates (12.5 μg/ml; TaKaRa Biotech, Japan) and centrifuged for 1.5 hours at 3000 g at 37° C. Following centrifugation, supernatant was removed and 10⁶ pre-stimulated T cells were added to the virus-coated plates. 24-48 hours later, T cells were removed from the plate and successful transduction was verified using flow cytometry. CAR expression was detected using fluorochrome-coupled anti-c-myc antibody (FITC, clone SH1-26E7.1.6, Miltenyi Biotec, Germany). The described experimental procedure for T cell culture and transduction is identical for all experiments provided herein.

### 5.2.4 Tumor Cell Line Culture

Human AML cell lines (THP-1, Mv4-11, OCI-AML, PL-21, U937, MOLM-13) were lentivirally transduced to express eGFP and fLuc and were cultured as described in Example 5.1.2.1.

### 5.2.5 AML Blast Isolation and Culture

For AML blast co-cultures, patient AML samples were thawed 3 days prior to starting the co-culture and cultured as described in Example 5.1.2.2.

### 5.2.6 Co-Culture of T Cells and Target Cells

For human co-culture experiments, 50.000 human AML cells were plated in a flat bottom 96 well plate. Tumor cells were co-cultured with transduced T cells or untransduced control T cells at the indicated effector to target cell ratio (E:T ratio) for 24 hours. All cells were resuspended in human T cell medium, not containing IL-2 or IL-15. CSF1R negative SU-DHL-4 cells were used as a negative control for CAR T cell-mediated killing. After 24 hours, T-cell mediated killing of AML cells were either determined using Bio-Glo™ Luciferase Assay System (Promega Corporation, USA) or flow cytometry. Flow cytometric-based determination of tumor cell death was quantified using Count Bright™ Absolute Counting Beads (Life Technologies, Darmstadt, Germany) after gating on GFP-positive tumor cells.

For co-cultures using primary human AML blasts, AML blasts were cultured as described in 5.2.5. On day 0, AML blasts were co-cultured either with allogenic T cells obtained from healthy donors or autologous T cells, isolated from PBMCs of AML patients following blast depletion. Autologous T cells were transduced as described above (5.2.3). Transduced CAR T cells or control T cells were co-cultured at the indicated effector to target cells ratios (E:T ratios). 48 hours later, lysis of AML blasts was determined by flow cytometry. T cells and AML blasts were grouped based on the expression of the T cell lineage marker CD2 and the myeloid marker CD33, highly expressed on AML blasts.

### 5.2.7 Assessment of T Cell Activation and T Cells Proliferation

Activation of T cells was determined by quantification of interferon gamma (IFN-γ) release following co-culture of T cells and tumor cells as described above. IFN-γ levels in supernatants of co-culture experiments were measured using human IFN-γ ELISA Kit (BD Bioscience, Germany). Measurements were carried out according to manufactures' protocol.

The following FACS antibodies were used to determine T cell proliferation (Example 5.2.6) and specific lysis (Example 5.2.7) in response to co-culture with human AML cells: anti-human CD2 (clone RPA-2.10, Biolegend, USA), anti-human CD3 (clone UCHT1, HIT3a, Biolegend, USA), anti-human CD4 (clone OKT4 Biolegend, USA), anti-human CD8 (clone SKI, HIT8a Biolegend, USA) and anti-human CD33 (clone P67.6, Biolegend, USA). Dead cells were identified with a fixable viability dye in all experiments (eFluor™ 780, eBioscience, USA). Proliferation was measured using Count Bright™ Absolute Counting Beads and gating for T cells was carried out using a panel of specific antibodies outlined above.

The following FACS antibodies were used to determine specific lysis in response to co-culture with primary AML blasts: anti-human CD3 (clone UCHT1, Biolegend, USA), anti-human CD4 (clone OKT4 Biolegend, USA), anti-human CD8 (clone SKI Biolegend, USA), anti-human CD33 (clone P67.6, Biolegend, USA; WM53, Invitrogen/eBioscience). Samples were analyzed using Beckman Coulter CytoFLEX.

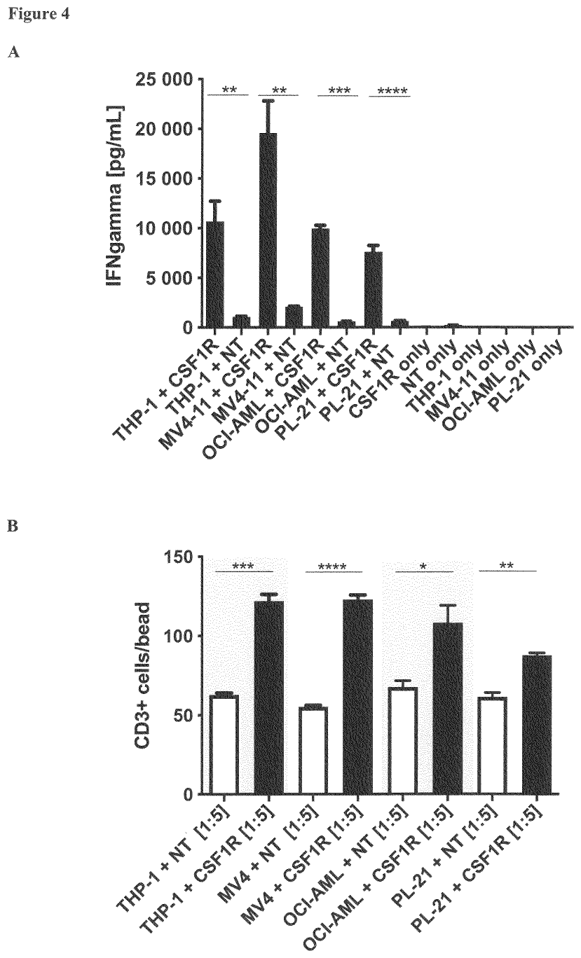

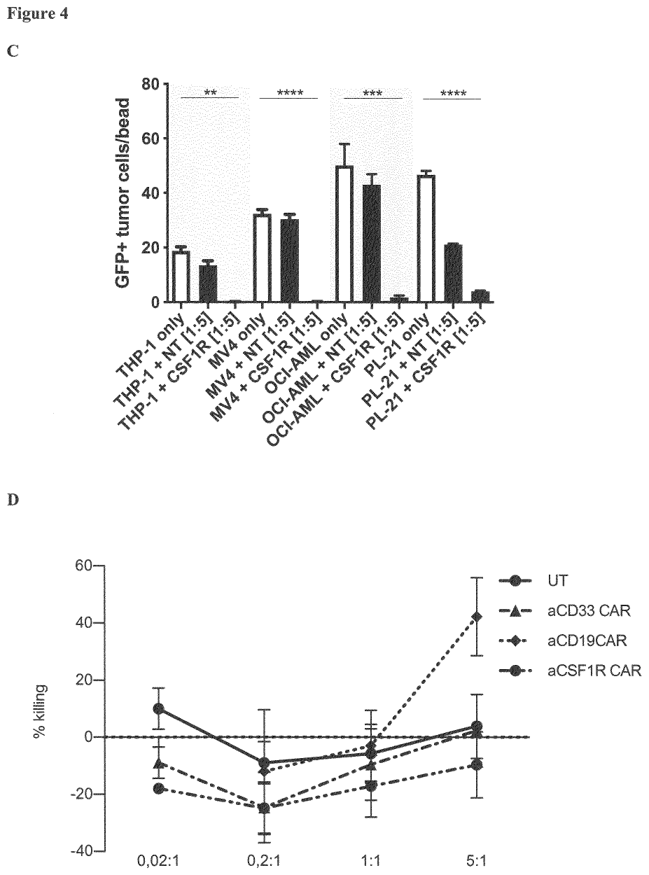

As shown in FIG. 4A, anti-CSF1R-CAR T cells showed significant activation as determined by IFN-γ release in the presence of human AML tumor cells as compared to cultures with non-transduced T-cells, T cells alone, and/or AML-cell lines alone. Furthermore, quantification of T cell counts by flow cytometry revealed significantly increased proliferation of anti-CSF1R CAR T cells in the presence of human AML cells as compared to non-transduced control cells (FIG. 4B). These results indicate that T cells are specifically activated by AML cells when expressing anti-CSF1R-CAR and that target recognition leads to sustained proliferation of anti-CSF1R CAR T cells.

### 5.2.8 CAR T Cell-Induced Target Cell Lysis

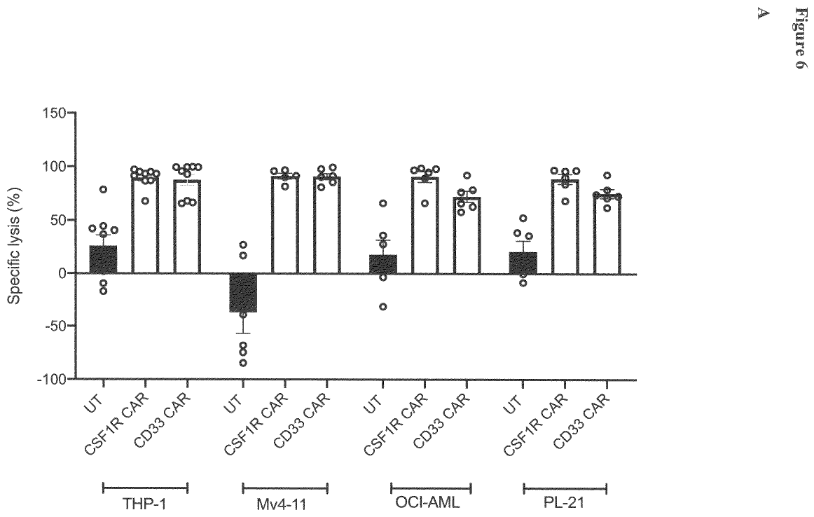

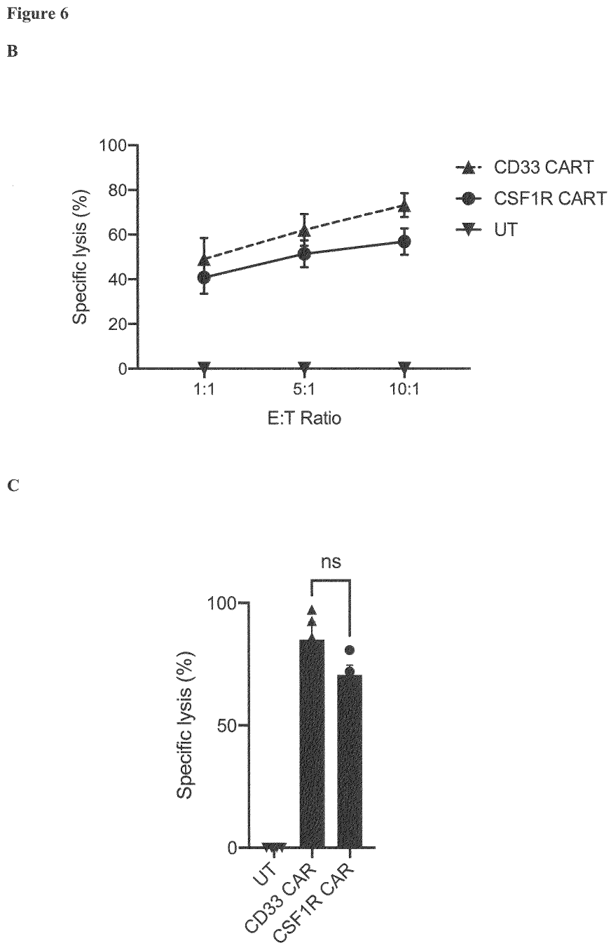

To verify that anti-CSF1R CAR T cells are able to lyse AML cell lines in vitro, co-culture experiments were carried out as described above. All experiments were carried out with fLuc-eGFP-expressing AML cells. Tumor cell lysis was determined either by flow cytometry or luminescence measurements following cell lysis in the presence of the fLuc substrate Luciferin as described. As shown in FIG. 4C, co-culturing of anti-CSF1R CAR T cells and AML cells revealed significant reduction in the number of detected GFP-positive tumor cells when compared to non-transduced T cells for all cell lines analyzed. Similarly, luminescence-based readout showed near 100% specific lysis of anti-CSF1R CAR T cells after co-culturing with human AML cells. In comparison to anti-CD33 CAR T cells, anti-CSF1R CAR T cells do not show significant differences, illustrating the potential for the use of anti-CSF1R CAR T cells for the treatment of AML (FIG. 6). Furthermore, it has been demonstrated that, despite the cell lines' heterogeneous expression of CSF1R (FIG. 3A), co-culture with the CAR T cell results in uniform CAR T cell activation as determined by cytokine release, target cell recognition and lysis.

Therapeutic effectivity of anti-CSF1R-CAR T cells was also demonstrated by determining T cell-specific lysis of primary AML blasts. Primary blasts were obtained from AML patients as described above and were co-cultured with allogenic transduced T cells expressing anti-CSF1R-CAR, anti-CD33-CAR T cells or non-transduced control T cells. As shown in FIGS. 6B and C, anti-CSF1R-CAR T cells transduced into allogenic (6B) or autologous T cells (6C) were able to specifically lyse primary AML Blasts and no significant difference to anti-CD33-mediated killing was determined.

### 5.2.9 Target Specificity of Anti-CSF1R-CAR

Finally, target specificity of anti-CSF1R-CAR T cells was examined by investigating non-specific T cell-induced tumor cell lysis. Transduced T cells expressing either anti-CSF1R-CAR or anti-CD19-CAR were co-cultured with CSF1R-negative, CD19-positive non-Hodgkin lymphoma cells SU-DHL-4. Again, as described above, cells expressed GFP and fLuc. SU-DHL-4 were cocultured with transduced T cells at the indicated E:T ratios for 48 hours. After 48 hours, cell lysis was determined using luminescence readout as illustrated above. As shown in FIG. 4D, anti-CD19-CAR T cells demonstrated efficient killing ofnon-Hodgkin lymphoma cells while no specific killing was induced by anti-CSF1R-CAR T cell or untransduced T cells. These results demonstrate a low off target rate and illustrate the specificity of our anti-CSF1R CAR T cells for its target.

### 5.3 Example 3: Comparison of CAR T Cells Specific for CD33 and CSF1R

The following example demonstrates the therapeutic effect of anti-CSF1R-CAR T cells as compared to anti-CD33-CAR T cells.

### 5.3.1 CAR Design and T Cell Culture

Anti-CSF1R-CAR was generated as described in Example 5.2.1. To compare the efficacy of the newly developed anti-CSF1R CAR T cells, the cells were compared to anti-CD33 CAR T cells. Generation of anti-CD33 CAR T cells has been previously described in Example 5.2.1.

### 5.3.2 AML Cell Line Culture and AML Blast Culture

AML cell lines were cultured as described above in Example 5.1.2.1.

### 5.3.3 Co-Culture of T Cells and Target Cells

Co-culturing of T cells and target cells were carried out as described in Example 5.2.5.

### 5.3.4 T Cell-Induced Target Cell Lysis

The therapeutic effect of anti-CSF1R-CAR T cells when compared to anti-CD33-CAR T cells was first investigated in vitro using established AML cell lines. Anti-CSF1R-CAR T cells and anti-CD33-CAR T cells were co-cultured with AML cell lines THP-1, MV4-11, OCI-AML or PL-21 as described above. T cell-induced lysis of AML cells was detected using Bio-Glo™ Luciferase Assay System (Promega Corporation, USA). As demonstrated in FIG. 6A, both anti-CSF1R-CAR T cells and anti-CD33-CAR T cells showed strong AML cell lysis when compared to untransduced control T cells. CSF1R-CAR T cells were able to efficiently lyse AML tumor cell lines similar to anti-CD33 CAR T cells highlighting the role for anti-CSF1R CAR T cells in the treatment of AML.

Therapeutic efficacy of anti-CSF1R-CAR T cells when compared to anti-CD33-CAR T cells was additionally investigated using AML primary blasts. Primary AML blasts were isolated and cultured as described in Example 5.1.2.2, and co-cultured with anti-CSF1R-CAR T cells or anti-CD33-CAR T cells for 48 h as described in Example 5.2.5. T cell-induced lysis was detected by using FACS analysis. As shown in FIG. 6B, co-culture revealed strong activation of both anti-CSF1R-CAR T cells and anti-CD33-CAR T cells as determined by specific lysis of AML blasts when compared to untransduced control T cells. No significant differences were observed between anti-CSF1R-CAR T cells and anti-CD33-CAR T cells.

Therapeutic effectivity of anti-CSF1R-CAR T cells as compared to anti-CD33-CAR T cells was additionally investigated in an in vitro cell model using autologous blasts and T cells from AML patients. Primary AML blasts were isolated and cultured as described in Example 5.1.2.2. T cells were isolated from the same patient and recombinantly engineered to express either the anti-CSF1R-CAR or the anti-CD33-CAR as described in Example 5.2.3. Autologous blasts and T cells were co-cultured for 48 h as described in Example 5.2.5, and T cell-induced lysis of primary AML blasts was detected by using FACS analysis as described in Example 5.1.2.3. Consistent with the results presented in FIGS. 6A and B, both anti-CSF1R-CAR T cells and anti-CD33-CAR T cells showed strong lysis of AML blasts when compared to untransduced control T cells (FIG. 6C). Thus, in summary, the ability of anti-CSF1R CAR T cells to lyse human AML cell lines and primary human AML blasts has been demonstrated, using both allogenic and autologous T cells.

### 5.4 Example 4: In Vivo Assays Demonstrating CSF1R as Therapeutic Target

The following example demonstrates CSF1R as targeting antigen as evaluated in in vivo models.

### 5.4.1 AML and T Cell Culture

Tumor cells and T cells were cultured as previously described in Examples 5.1.2.1 and 5.2.3.

### 5.4.2 AML Mouse Model

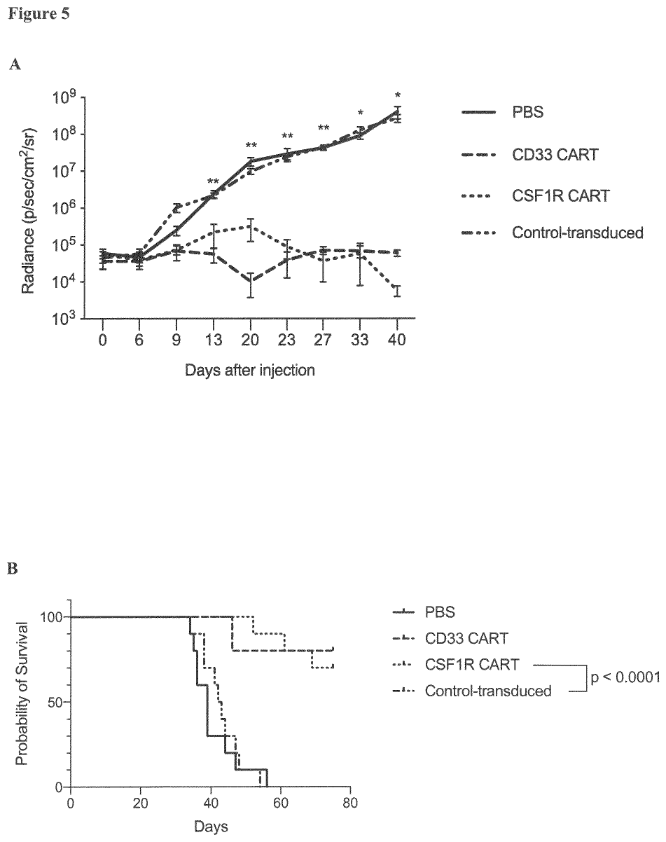

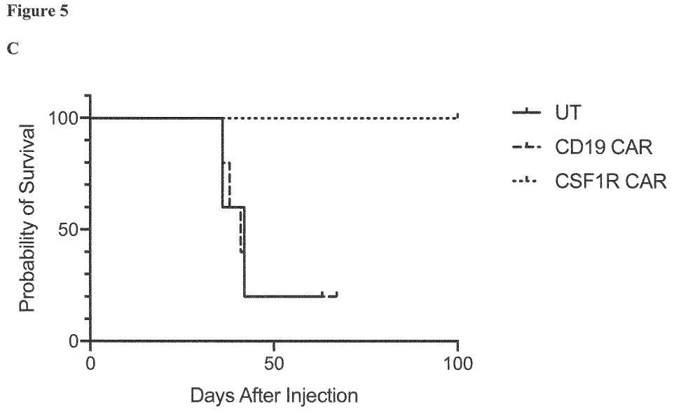

In vivo therapeutic efficacy of anti-CSF1R-CAR T cells was explored in cell line-derived xenograft (CDX) mouse models and a patient-derived xenograft model (PDX). For the CDX models, commercially available human AML cell lines MV4-11 (FIG. 5A) or THP-1 (FIG. 5B) served as xenograft for implantation into immunodeficient mice. 10⁶ MV4-11 or THP-1 cells expressing eGFP and fLuc were injected intravenously (i.v.) into immunodeficient NOD.Cg-Prkdc^{scid} Il2rg^{tm1WjI}/SzJ (NSG, stock number 005557) mice. Mice were purchased from Charles River (Sulzfeld, Germany) or bred within the local animal facility (Zentrale Versuchstierhaltung, Innenstadt, Munich, Germany). All conducted animal experiments were approved by the local regulatory agency (Regierung von Oberbayern). Tumor growth was monitored using the In vivo Imaging System Plattform Lumina X5 (IVIS, PerkinElmer, USA) after intraperitoneal (i.p.) injection of substrate (Xenolight D-Luciferin potassium salt, Perkin Elmer, USA) into each mouse according to manufacturer's instructions. Afterwards, mice were i.v. treated with PBS or 10⁶ T cells expressing anti-CSF1R CAR, anti-CD33 CAR or a control construct harboring the same CD28 co-stimulatory and CD3ζ signaling domain. For PDX models (FIG. 5C), primary AML samples lentivirally transduced to express luciferase were thawed and injected into NSG mice as described in Vick et al. (PLoS One (2015) 20; 10(3):e0120925). For PDX models, mice were treated with either untransduced T cells, anti-CSF1R CAR T cells or anti-CD19 CAR T cells as a negative control. Transduction efficiencies for each experiment were around 40-60%.

As shown in FIG. 5A, in CDX models, treatment with anti-CSF1R-CAR T cells results in a decrease in luminescence signal due to induction of a strong anti-tumor immune response when compared to mice treated with either PBS or control-transduced T cells. Anti-tumor activity of the administered T cells also correlated with increase in survival of recipient mice (FIG. 5B). Similarly, as shown in FIG. 5C, in a PDX model (FAB: M4; Cytogenetics: t(6;11)(q27;q23) KMT2A/AFDN (=MLL-AF6)), anti-CSF1R CAR T cells induced a strong anti-tumor response, leading to complete tumor clearance in all treated mice. In comparison, mice treated either with untransduced T cells or anti-CD19 CAR T cells died of disease progression. Mice treated with anti-CSF1R CAR T cells stayed tumor free over a follow-up period of up to 100 days.

### 5.5 Example 5: Treatment of AML Using CSF1R CAR T Cells

Anti-CD33 CAR T cells are highly effective but often present with serious adverse effects such as severe hematotoxic and neurotoxic side effects. Having proven the potential of anti-CSF1R CAR T cells in vitro and in vivo, potential side effects of the newly developed anti-CSF1R-CAR T cells were determined. As the most common side-effects of CAR T cells therapies in hematological malignancies are on-target off-tumor toxicities and the development of neurotoxicities, it was primarily focused on these two major adverse effects.

### 5.5.1 Search of Public Databases

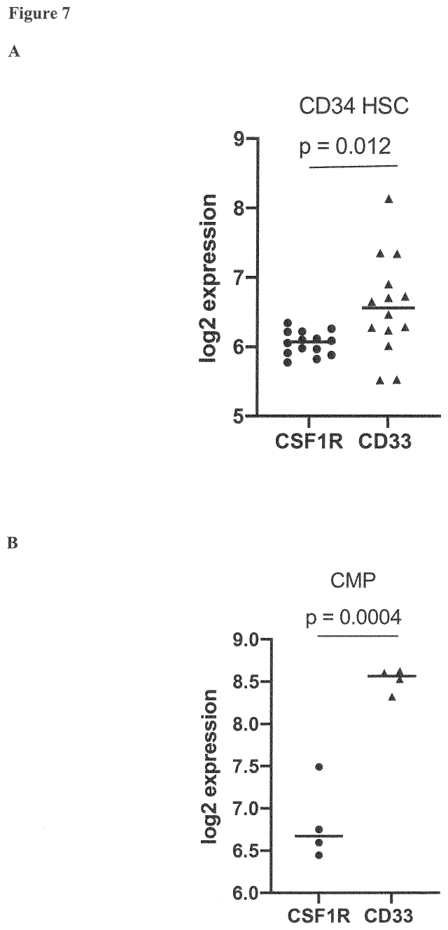

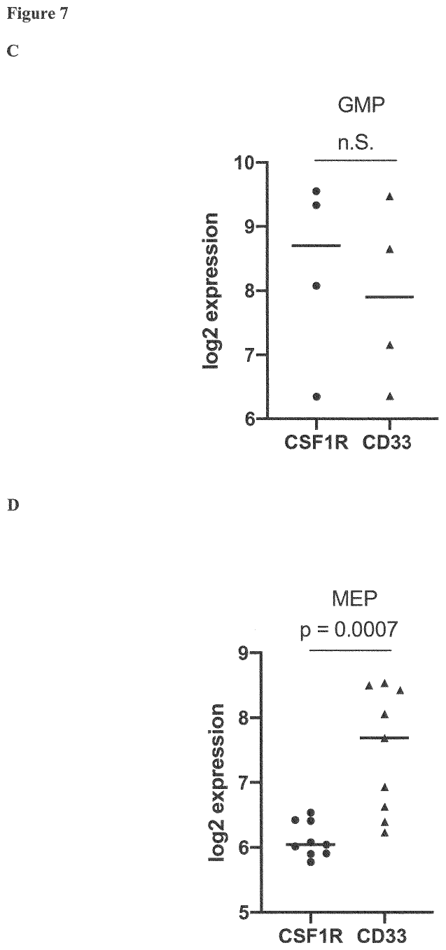

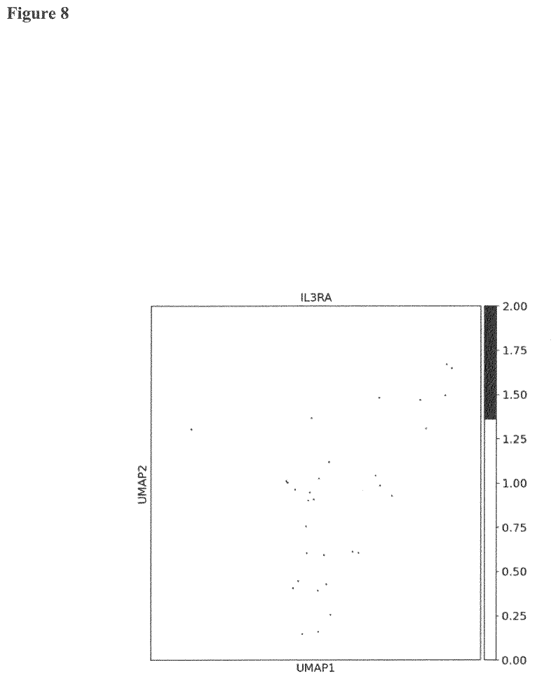

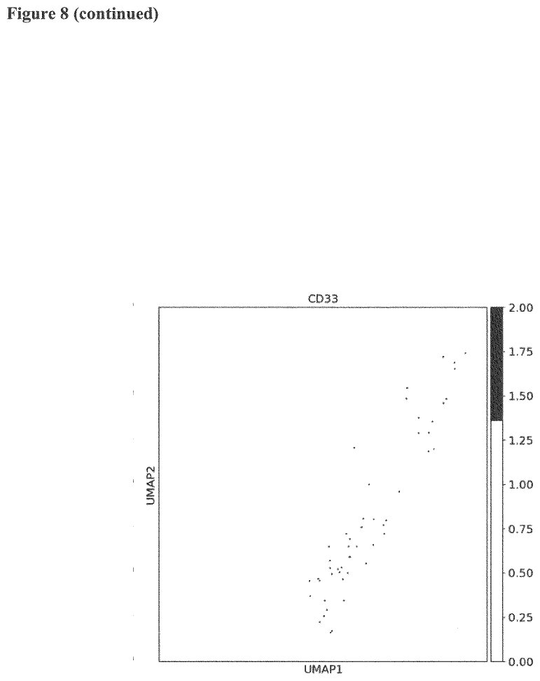

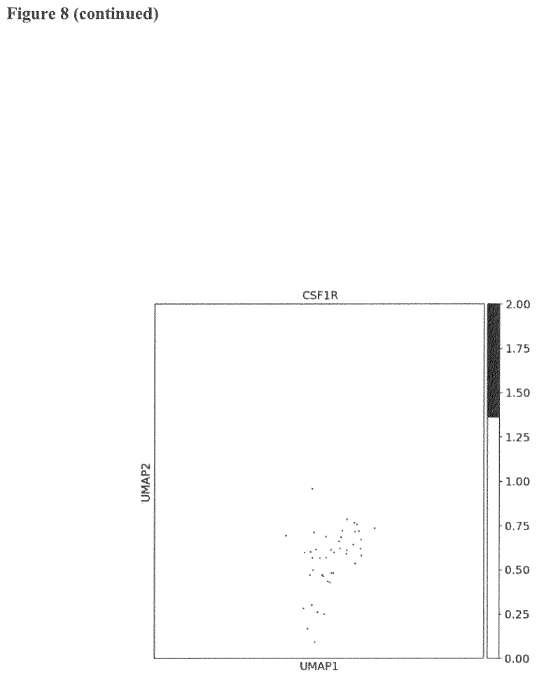

To assess potential off-tumor reaction of anti-CSF1R CAR T cells, the expression pattern of CSF1R on hematopoietic stem cells (HSC), hematopoietic progenitor cells (HPC) and mature immune cells using either bulk sequencing data or single cell sequencing data were analyzed. Thus, expression of CSF1R and CD33 was analyzed on CD34-positive hematopoietic stem cells (HSC), common myeloid progenitor cells (CMP), granulocyte/monocyte progenitor cells (GMP) and megakaryocyte/erythroid progenitor cells (MEP) using BloodSpot database. BloodSpot is a public, gene-centric database of mRNA expression of haematopoietic cells using bulk RNA Sequencing. As shown in FIG. 7 A-D, BloodSpot analysis revealed equal expression of CSF1R and CD33 on GMP cells. Remarkably, expression of CSF1R was found to be significantly lower on HSC, CMP and MEP cells when compared to CD33 expression. These results indicate CSF1R to be a more specific marker antigen for AML when compared to CD33. Furthermore, single cell RNA sequencing was used to validate the hypothesis. As illustrated in FIG. 8, scRNA Seq revealed significantly lower expression on HSC and HSPCs than the two major AML target antigens CD33 and CD123. The reduced expression of CSF1R on HSCPs hold the promise that CSF1R-directed therapies will spare human hematopoietic stem cells and thus be less hematotoxic.

### 5.5.2 Cell Culture Hematopoietic Stem Cells

Cord blood (CB)- or bone marrow (BM)-derived human CD34+ stem cells were obtained from Stemcell Technologies. All cells were collected after informed consent in accordance with the Declaration of Helsinki. CB CD34+ cells were thawed in a pre-warmed water bath at 37°. Directly after thawing, cells were expanded using StemSpan II Medium (Stemcell Technologies, Vancouver, Canada), supplemented with serum-free nutrient supply and UM729 small molecule inhibitor. For HSC assays and FACS analysis, cells were expanded a total of 7 days, medium was changed after 3 days.

### 5.5.3 FA CS Expression Analysis

To confirm that CSF1R is a more specific and improved marker for AML as compared to CD33, the expression of CSF1R and CD33 by CD34+ and CD38-negative HSC and by CD34-positive, CD38-positive HPC was determined by FACS. Stem cells were purchased and cultivated as described in Example 5.5.2. FACS analysis was carried out as described in 5.1.2.3.

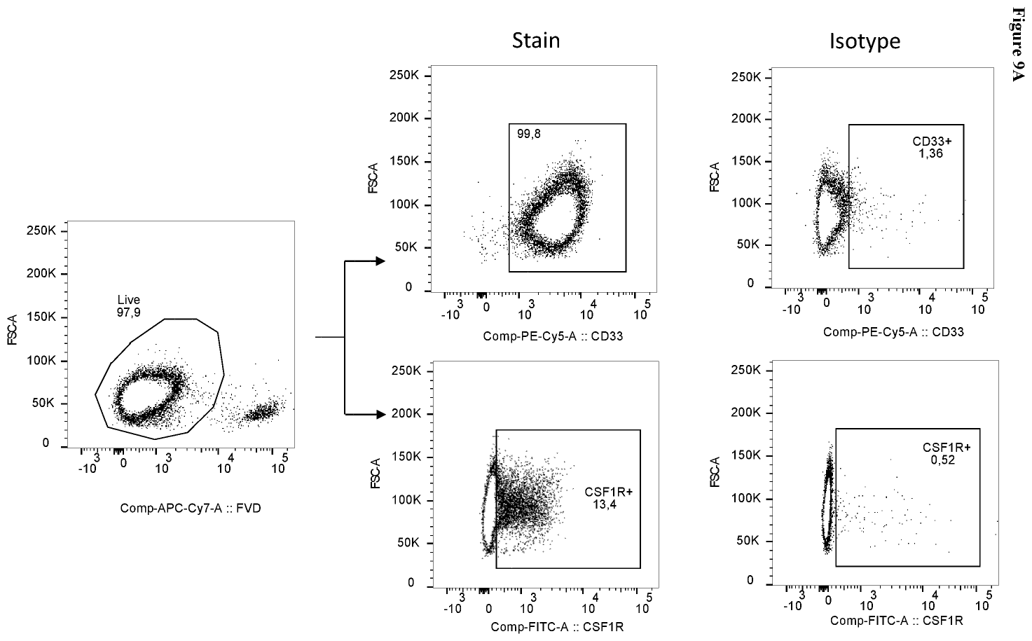

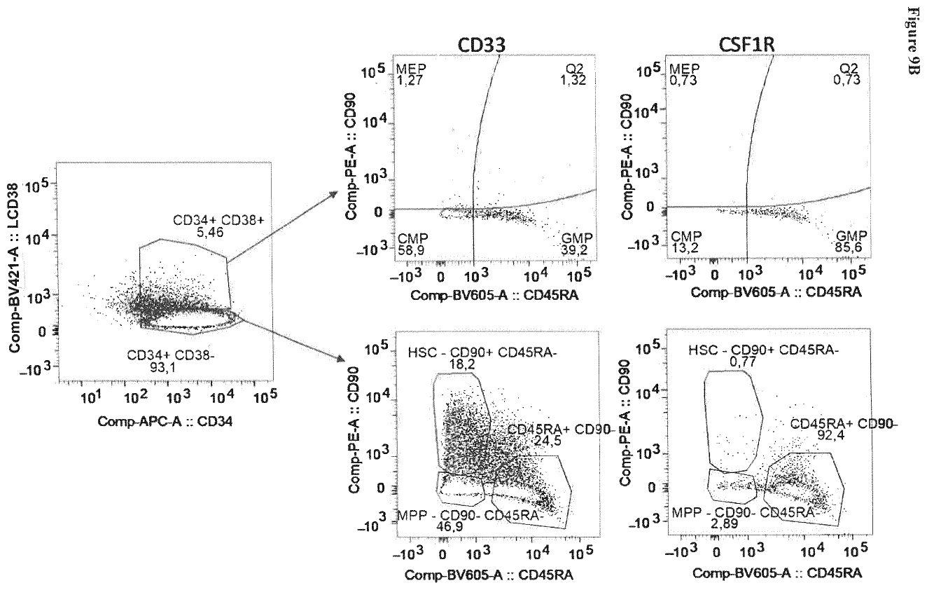

The following FACS antibodies were used for expression analysis of HSCs (FIG. 9): anti-human CD33 (clone WM53, Biolegend, USA), anti-human CD34 (clone 561, Biolegend, USA), anti-human CD38 (clone HB-7, Biolegend, USA), anti-human CD45 (clone HI30, Biolegend, USA), anti-human CD45RA (clone HI100, Biolegend, USA), anti-human CD90 (clone 5E10, Biolegend, USA) anti-human CD115 (clone 9-4d2-1e4, Biolegend, USA). Samples were analyzed using BD LSRFortessa™ II. Dead cells were excluded after staining with a fixable viability dye (eFluor™ 780, eBioscience, USA).

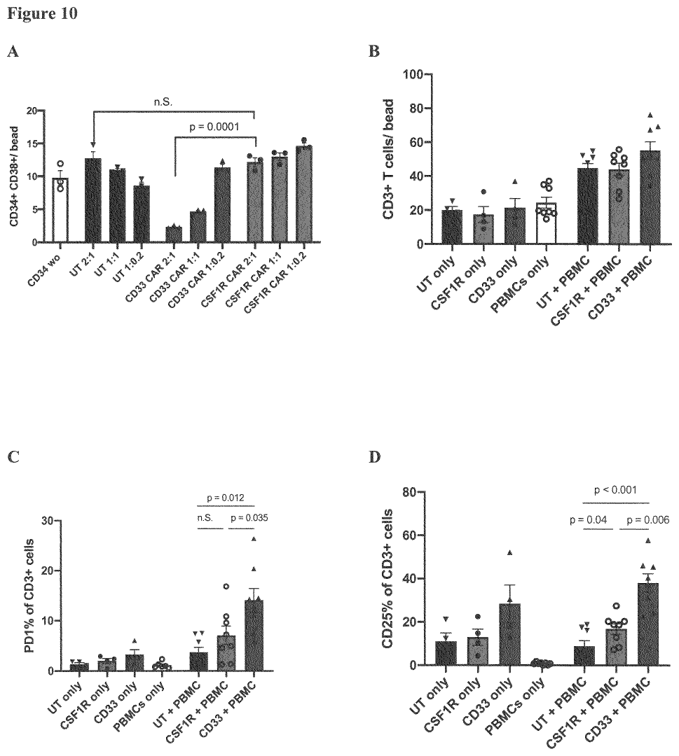

The following FACS antibodies were used for co-culture experiments with CAR T cells and human HSC as described in Example 5.6.3 (FIG. 10): anti-human CD3 (clone HIT3a, Biolegend, USA) anti-human CD33 (clone WM53, Biolegend, USA), anti-human CD34 (clone 561, Biolegend, USA), anti-human CD38 (clone HB-7, Biolegend, USA), anti-human CD45RA (clone HI100, Biolegend, USA), anti-human CD90 (clone 5E10, Biolegend, USA) anti-human CD115 (clone 9-4d2-1e4, Biolegend, USA). Samples were analyzed using BD LSRFortessa™ II. Dead cells were excluded after staining with a fixable viability dye (eFluor™ 780, eBioscience, USA).

As shown in FIGS. 9A and B, CSF1R was only expressed on a small subset of cells (13.4% of live cells), while CD33 was very broadly expressed (99.8% of live cells). When taking a more detailed look into CSF1R and CD33 expressing subsets, it was found that CSF1R was only expressed in a small subset of HSPC. In line with the RNA analysis (Example 5.5.1), CSF1R was mostly expressed on CD34+CD38+ GMPs and only expressed on CD45RA+CD90-HSCs. In comparison, CD33 was homogenously expressed across different HSC subsets as well as strongly expressed on CMP and GMP. Thus, targeting CSF1R in AML can potentially spare the earliest progenitors of human stem cells, which carry out essential functions to sustain human hematopoiesis. For this reason, anti-CSF1R CAR T cells, compared to e.g. anti-CD33 CAR T cells, have the potential to minimize suppression of human hematopoiesis.

### 5.6 Example 6: Target Specificity of Anti-CSF1R-CAR T Cells

The following example demonstrates the target specificity of anti-CSF1R-CAR T cells as compared to anti-CD33-CAR T cells.

### 5.6.1 T Cell Culture

Anti-CSF1R-CAR and anti-CD33-CAR T cells were generated as described in Example 5.2.3.

### 5.6.2 Cell Culture

Human CD34+BM- or CB-derived hematopoietic stem cells were obtained as described in Example 5.5.2. PBMC were isolated from healthy donors using density centrifugation (see Example 5.2.3). Healthy human bone marrow samples were obtained from patients undergoing hip replacement surgery after written informed consent in accordance with the Declaration of Helsinki and approval by the Institutional Review Board of the Ludwig-Maximilians Universitat (Munich, Germany). Long-term co-cultures of CAR T cells and healthy bone marrow samples were conducted in a similar fashion as co-cultures with primary AML blasts and CAR T cells (see Example 5.1.2.2)

### 5.6.3 Co-Culture of T Cells and Target Cells

For co-culture of T cells and HSPC, anti-CD33, anti-CSF1R CAR T cells or untransduced T cells were mixed with human BM-derived CD34+ cells to a final volume of 200 l per well in a flat bottom 96 well plate in an effector:target cell ratio as indicated in the respective FIG. 10A. All cells were cultured in IMDM containing 2% FCS and 0.5% penicillin streptomycin. After 48 hours, target cell lysis was determined using FACS (see Example 5.5.3).

For co-culture of T cells and PBMCs, 50.000 CAR T cells or untransduced T cells were mixed with donor matched PBMCs in human T cell medium (see Example 5.2.3) in an effector:target cell ratio of 1:2 and cultured in 96 well flat bottom plates. Cells were co-cultured for 48 h prior to FACS analysis.

The following FACS antibodies were used for co-culture experiments of CAR T cells and human PBMC (Example 5.6.3, FIG. 10 B-D): anti-human CD3 (clone HIT3a, Biolegend, USA), anti-human CD11b (clone IcRF44, Biolegend, USA), anti-human CD11c (clone 3.9, Biolegend, USA), anti-human CD14 (clone 561, Biolegend, USA) anti-human CD25 (clone BC96, Biolegend, USA), anti-human CD14 (clone M5E2, Biolegend, USA), anti-human CD19 (clone HIB19 Biolegend, USA), anti-human CD33 (clone WM53, Biolegend, USA), anti-human CD56 (clone HCD56, Biolegend, USA), anti-human CD115 (clone 9-4d2-1e4, Biolegend, USA). anti-human CD297 (PD-1, EH12.2H7, Biolegend, USA) Samples were analyzed using BD LSRFortessa™ II. Dead cells were excluded after staining with a fixable viability dye (eFluor™ 780, eBioscience, USA).

For co-culture of T cells and bone marrow cells, wells of a 96 well plate were precoated with a feeder layer of irradiated MS-5 stromal cells as described in Example 5.1.2.2. The medium was aspirated and 300.000 bone marrow cells were mixed with CAR T cells or untransduced T cells to a final volume of 200 μl per well in an effector:target cell ratio of 1:5 and 1:10 (see Example 5.2.5). Before plating, cells were resuspended in a cytokine rich medium (see Example 5.1.2.2). Cells were co-cultured for 3 or 6 days in cytokine medium prior to FACS analysis. FACS staining, antibodies and analysis was carried out as described in Example 5.2.6.

### 5.6.4 FA CS Analysis

Target specificity of anti-CSF1R CAR T cells when compared to anti-CD33 CAR T cells was assessed by determining T cell-mediated killing of HSPC. T cells were isolated and genetically modified to express either anti-CSF1R-CAR and anti-CD33-CAR as described in Example 5.2.3. HSPC and transduced or untransduced T cells were co-cultured for 48 h as described in Example 5.6.3, and T cell-mediated killing was measured by FACS analysis. To quantify the cell numbers, Count Bright™ Absolute Counting Beads were used as described in Example 5.5.2. As shown in FIG. 10A, anti-CSF1R CAR T cells show lower killing of HSPC when compared to anti-CD33 CAR T cells.

Furthermore, target specificity of anti-CSF1R CAR T cells as compared to anti-CD33 CAR T cells was investigated by determining activation and exhaustion after co-culture with donor-matched PBMCs from healthy subjects. T cells were isolated and recombinantly modified to express either anti-CSF1R-CAR or anti-CD33-CAR as described in Example 5.2.3. PBMCs and transduced or untransduced T cells were co-cultured for 48 h as described in Example 5.6.3, and activation and exhaustion of T cells was detected by quantification of CD25+, PD1+ and CD3+ cells per bead using FACS analysis. As shown in FIG. 10 B-D, anti-CSF1R CAR T cells exhibit significantly fewer signs of activation and exhaustion than anti-CD33 CAR T cells. As T cell activation is accompanied by excessive secretion of pro-inflammatory cytokines, which can lead to serious adverse effects, such as cytokine release syndrome (CRS) or immune effector cell-associated neurotoxicity syndrome (ICANS), treatment with anti-CSF1R CAR T cells could potentially be associated with reduced incidence rates of both CRS and ICANS and thus would be a more safe therapeutic option than anti-CD33 CAR T cells.

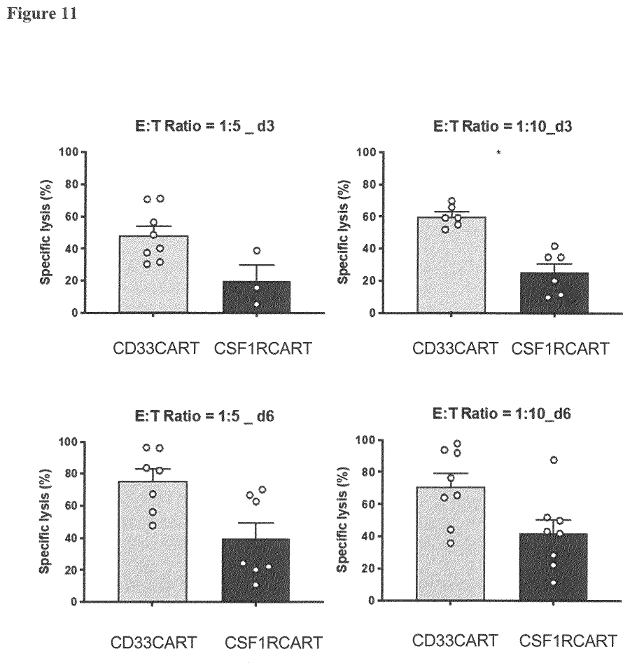

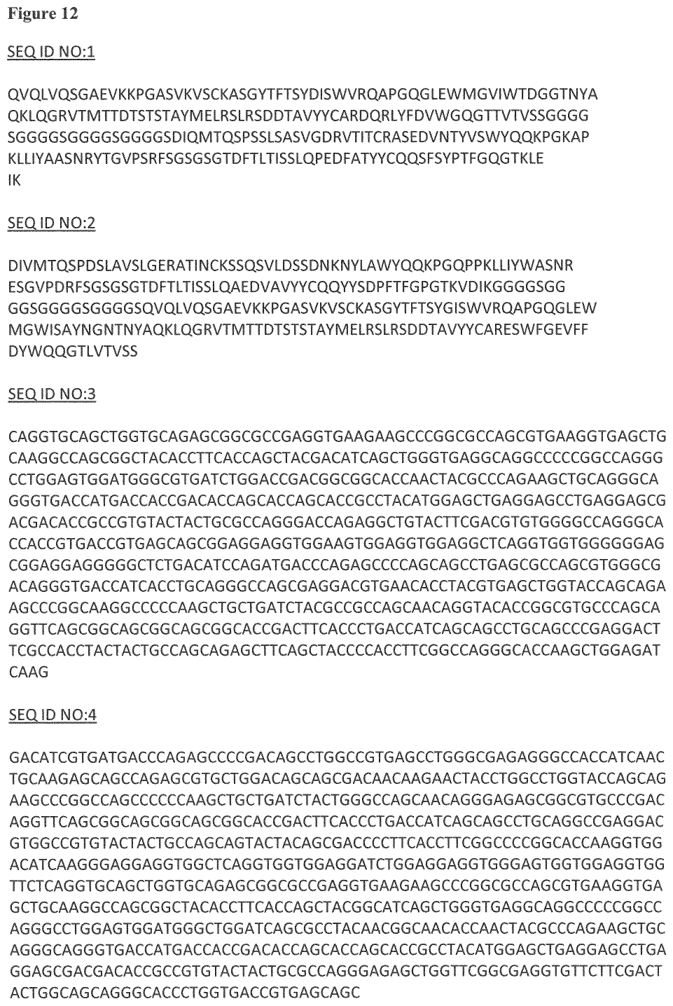

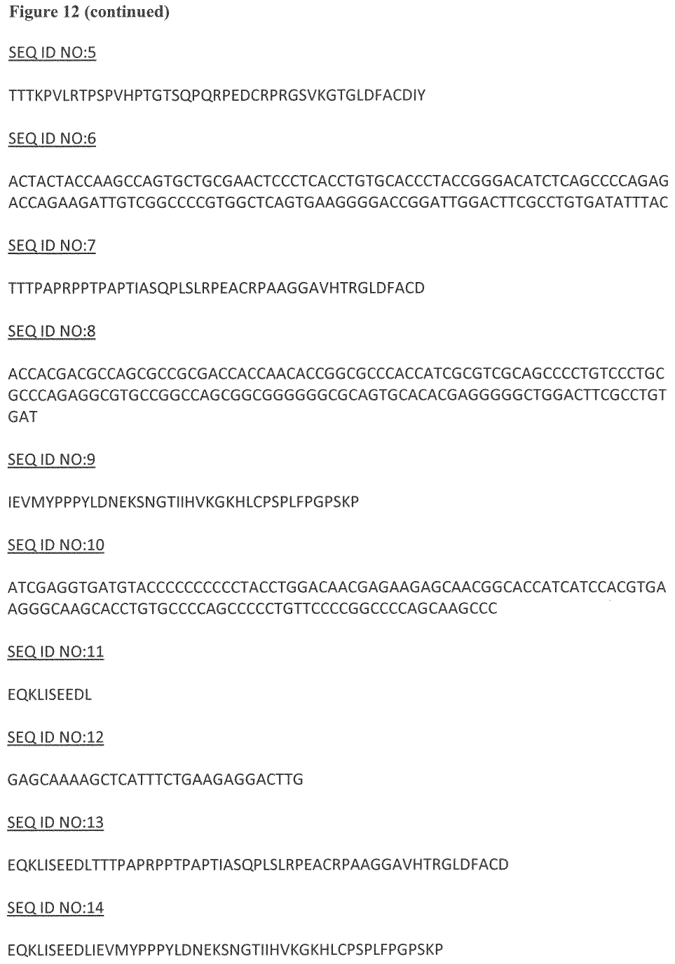

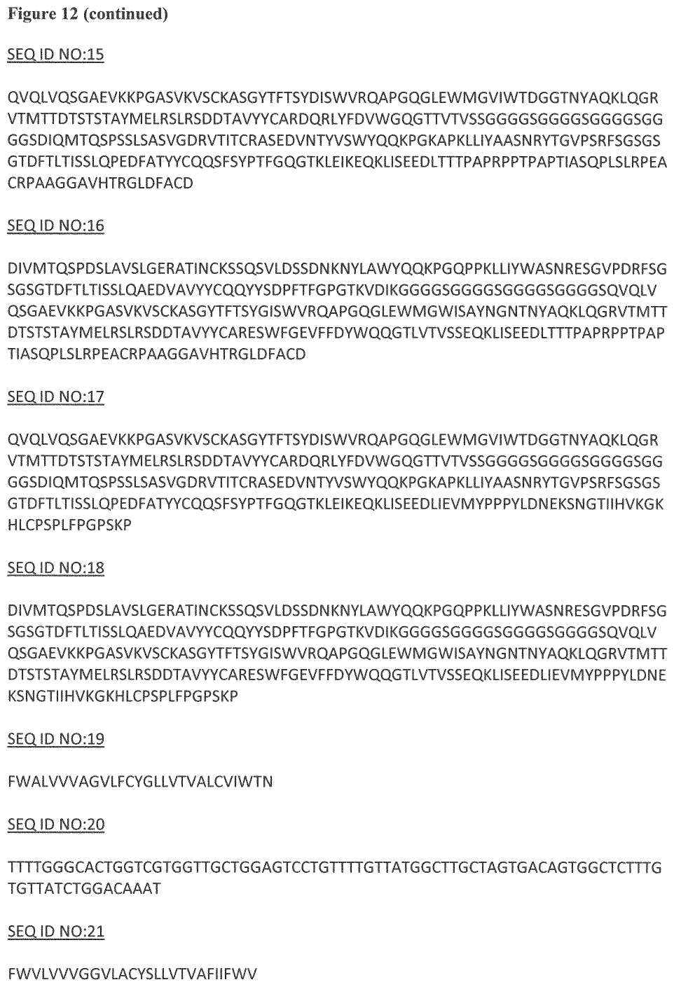

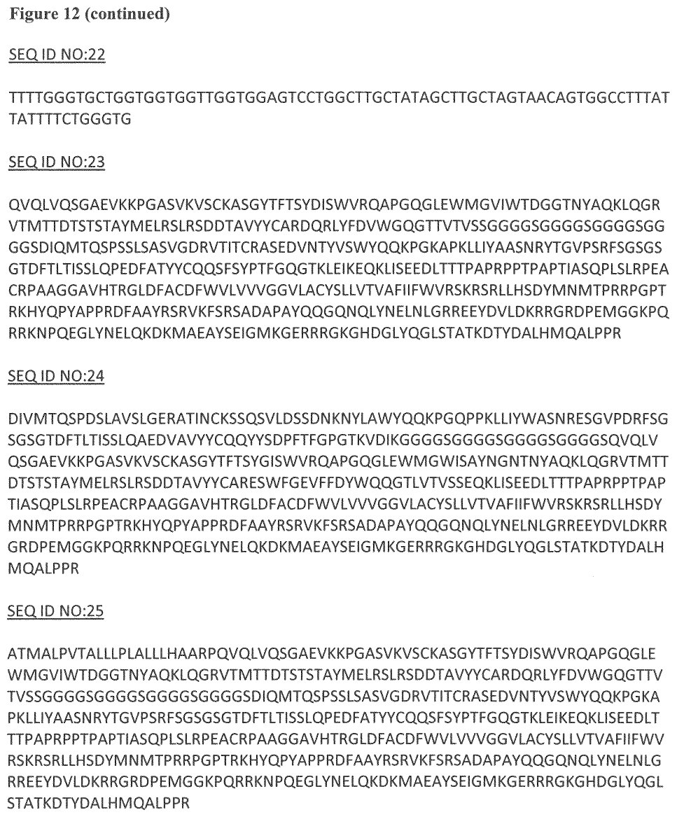

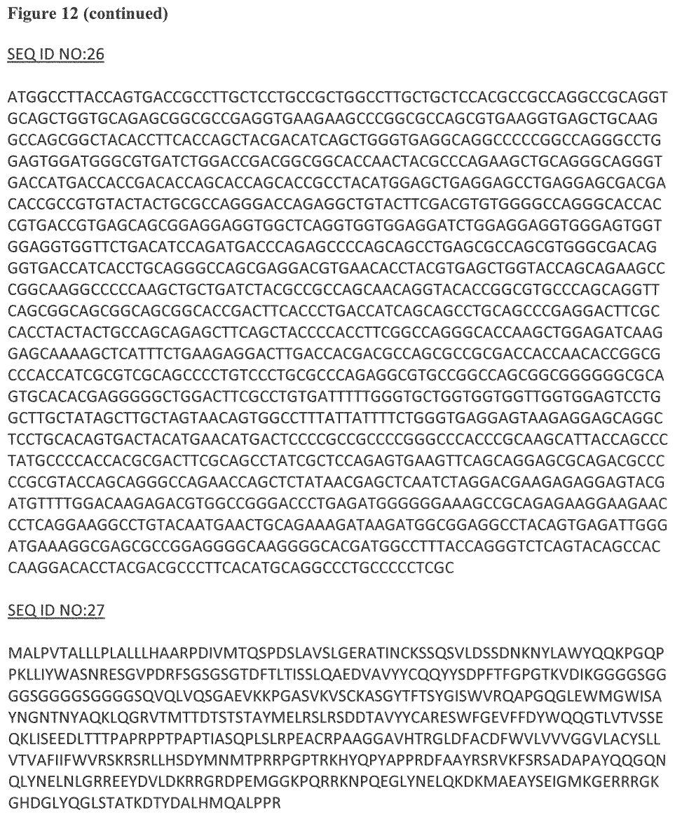

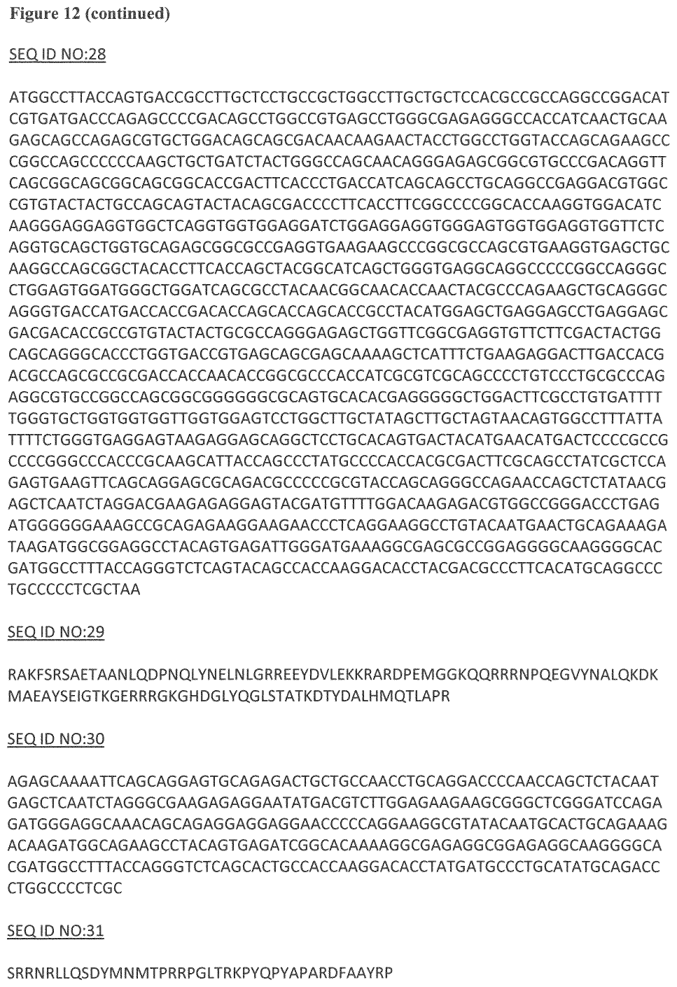

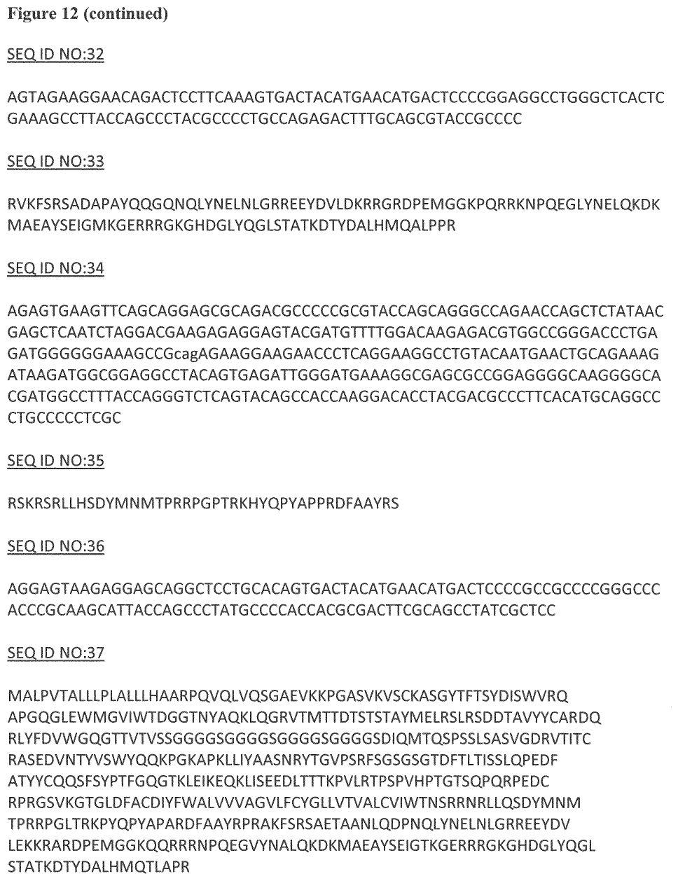

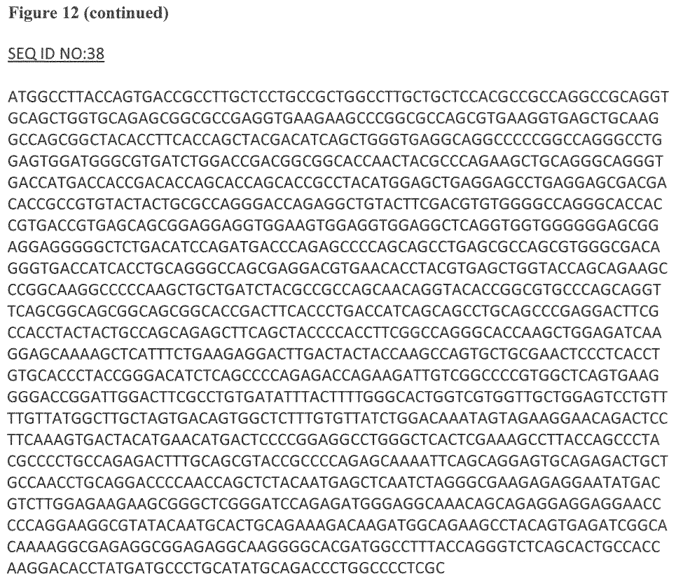

Target specificity of anti-CSF1R CAR T cells as compared to anti-CD33 CAR T cells was also investigated by determining specific T cell-induced lysis of healthy bone marrow cells. T cells were isolated and genetically modified to express either anti-CSF1R-CAR or anti-CD33-CAR as described in Example 5.2.3. PBMCs and transduced or untransduced T cells were co-cultured for 72 h as described in section 5.6.3 and above. As shown in FIG. 11, anti-CD33-CAR T cells lyse healthy bone marrow cells to a greater extent than anti-CSF1R-CAR T cells. These results suggest CSF1R to be a more specific target antigen than CD33 for the treatment of AML and that treatment with anti-CSF1R CAR T cells do not lead suppress the bone marrow function as strongly as anti-CD33 CAR T cells.

### 5.7 Example 7 CSF1R Expression in Samples of AML

The following example further confirms the results of Example 1, demonstrating CSF1R as suitable target antigen for treating AML.

### 5.7.1 Additional Search of Public Databases

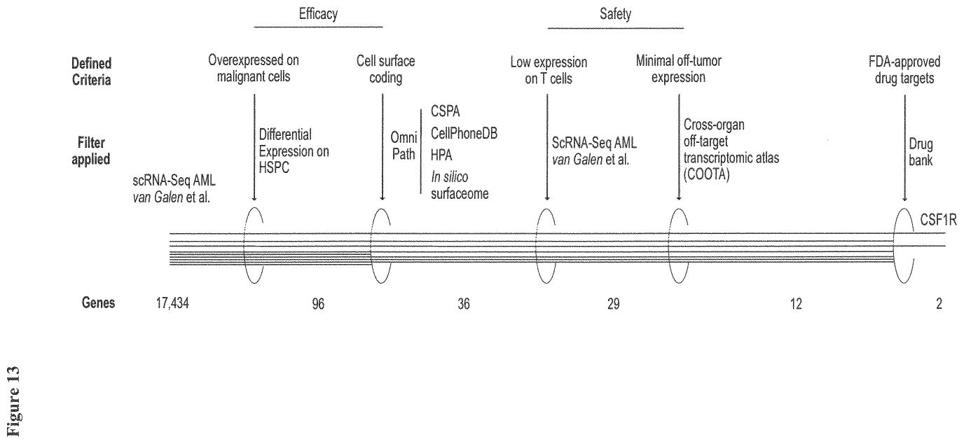

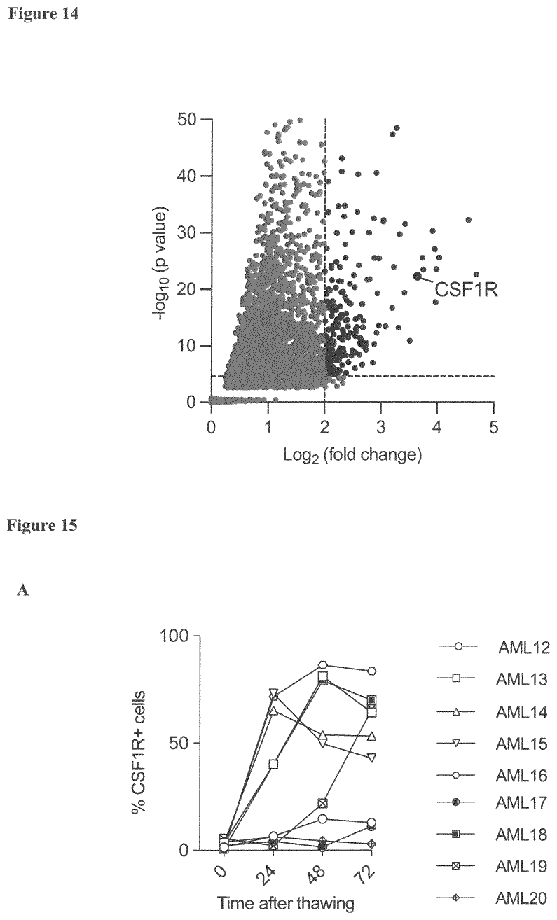

In addition to the described search for suitable target antigens for treating AML using the public databases GEPIA and bloodspot.eu as described in Example 5.1.1, which leverage public bulk RNA-sequencing data, comprehensive, single-cell RNA-sequencing (scRNA-seq)-based target screening analysis was conducted (FIG. 13). Single-cell sequencing strategies, in comparison to conventional bulk sequencing analysis, are able to predict expression pattern at a much higher resolution as cell-type specific expression patterns are analyzed (Zheng et al., Nat Commun. (2017); 8:14049. These methods have so far not been used for de novo target prediction. Using complex harmonization procedures of 12 different single cell datasets, an unbiased screening algorithm was built (FIG. 13). Single cell datasets were obtained from Stewart et al., Science. (2019); 365(6460):1461-6, Travaglini et al., Nature (2020); 587(7835):619-25, Habib et al., Nat Methods. (2017); 14(10):955-8, Han et al., Nature (2020); 581(7808):303-9, James et al., Nat Immunol. (2020); 21(3):343-53, Kim et al., Nat Commun. (2020); 11(1):2285, MacParland et al., Nat Commun. (2018); 9(1):4383, Madissoon et al., Genome Biol. (2019); 21(1):1, Ramachandran et al., Resolving the fibrotic niche of human liver cirrhosis at single-cell level. Nature. (2019); 575(7783):512-8, Reyfman et al., Am J Respir Crit Care Med. (2019); 199(12):1517-36, van Galen et al. Cell. (2019); 176(6):1265-81 e24. The algorithm used a multi-step approach to identify possible target antigens: First differential gene expression analysis between malignant and healthy hematopoietic stem and progenitor cells (HSPC) was performed. Genes which were significantly overexpressed on malignant HSPC, compared to their healthy counterpart (which would allow for selective lysis of malignant cells) were then filtered for surface expression, as only antigens which are expressed on the cell surface would be suitable for CAR therapy. Next, genes which were highly expressed on T cells were excluded from the analysis, as high T cell expression would limit the in vivo proliferation of CAR T cells and can lead to fratricide during the production of the CAR products. Finally, to minimize on-target off-tumor expression of the newly identified target antigens, targets, which were highly expressed on healthy tissues of nine different healthy organs were excluded. To add another safety level to the analysis, targets of FDA-approved drugs were specially considered, as these antigens have already proven to be safe in clinical trials. Using rigorous cut-offs for each level of the multistep algorithm, CSF1R was identified as only one of two possible target antigens for CAR therapy in AML (FIG. 13 and FIG. 14).

These comprehensive analyses using both bulk and scRNA-seq analysis unambiguously identify CSF1R as a promising candidate for immunotherapy in AML and confirm the results presented in Example 5.1.

### 5.7.2 Additional Analysis of CSF1R Expression in Patient Samples of AML Blasts and in AML Cell Lines

AML blast isolation, culture and FACS analysis were conducted as described in Examples 5.1.2.2 and 5.1.2.3. Specifically, primary AML samples were cultured on irradiated MS-5 (murine bone marrow stromal cells) for co-culture experiments as previously described in Example 5.1.2.2 and 5.1.2.3 (Benmebarek et al., Leukemia. (2021), van Gosliga et al., Exp Hematol. (2007); 35(10):1538-49, and Herrmann et al., Blood. (2018); 132(23):2484-94). For FACS analysis, CSF1R was stained after incubation with biotinylated recombinant CSF-1 protein (Sino Biological, China) followed by secondary staining with Streptavidin APC (BioLegend, USA).

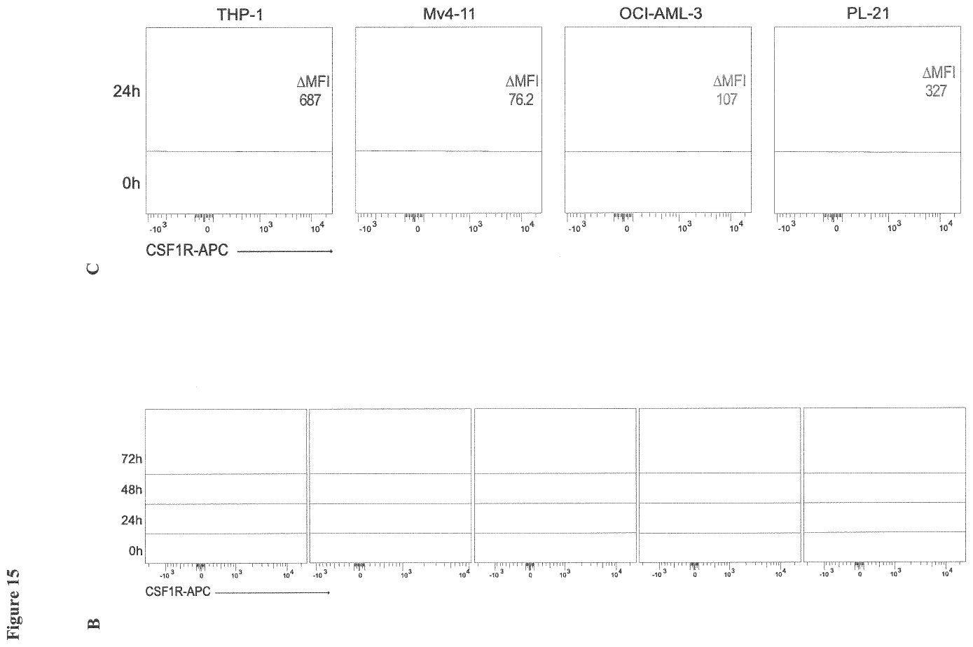

As the data contradict current literature, the expression of CSF1R on primary AML cells and AML cell lines following thawing of these cells was analyzed. Primary AML samples are usually obtained from bone marrow aspirates, frozen and stored in the liquid nitrogen at the respective institution for long term preservation. No CSF1R expression was observed directly after thawing of the primary AML blasts (FIG. 15A, B, time point 0), but was highly detectable after at least 24 hours of culture (FIG. 15A, B). To prove, that these changes in expression are due to artifacts caused by the freezing and thawing procedures, CSF1R+ AML cell lines as additional models were used (FIG. 15C). In line with the previous results, CSF1R was not expressed on AML cell lines directly after thawing, but detectable after 24 hours of culture (FIG. 15C).

These analyses demonstrate that CSF1R is indeed highly expressed on primary AML blasts and that until now, true frequency of CSF1R expression on primary samples was underestimated, most likely due to artifacts caused by freeze-thaw cycles of primary AML cells and AML cell lines, highlighting the innovative nature of the herein described results.

### 5.8 Example 8 In Vivo Assays Demonstrating CSF1R as Therapeutic Target

The following example confirms the results of Example 4 which demonstrates CSF1R as a therapeutic target.

### 5.8.1 AML Mouse Model

The experiment was conducted as described in Experiment 5.4.2. Specifically, OCI-AML3 expressing eGFP and fLuc were injected as previously described for CDX models with AML tumor cell lines THP-1 and Mv4-11. PDX models were used as previously described.

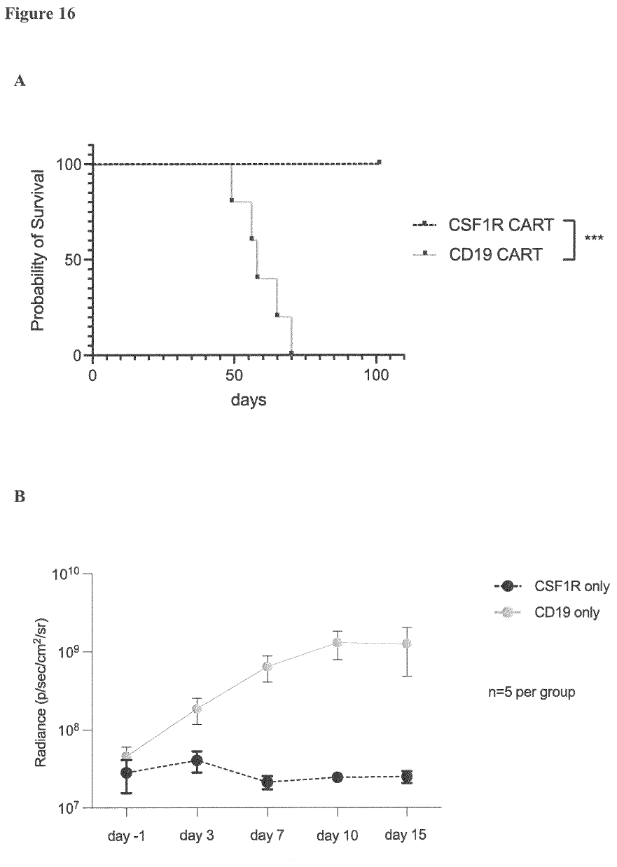

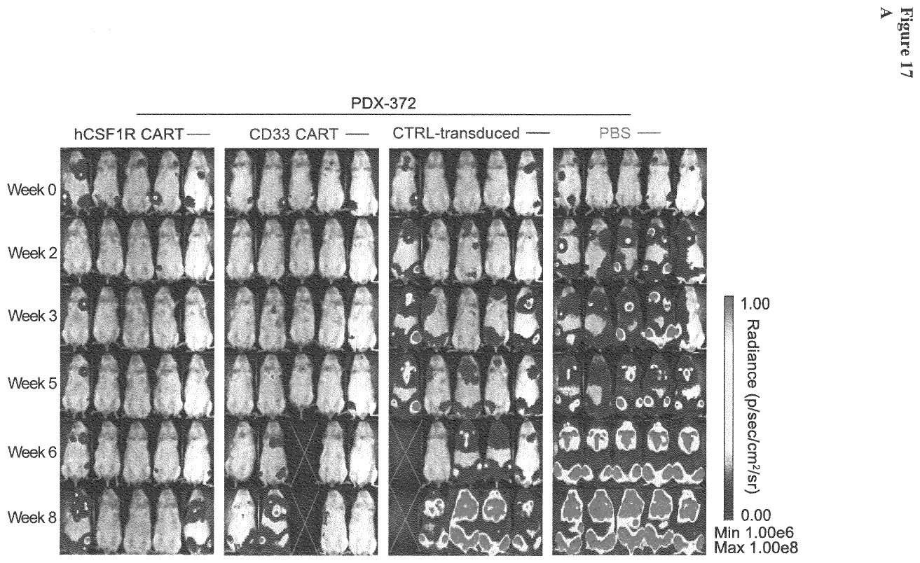

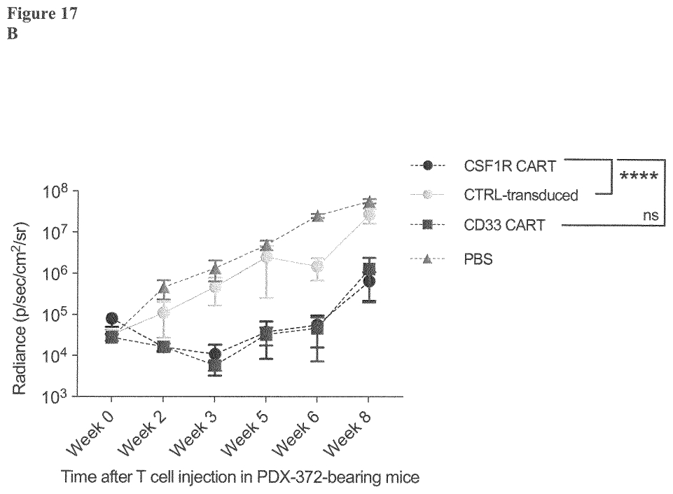

Due to the heterogeneity of AML as a disease, spanning a multitude of different cytogenetics aberrations, in vivo analysis of treatment efficacy should be carried out in several different CDX and PDX models. Thus, to proof functionality of anti-CSF1R-CAR T cells in another CDX model, OCI-AML3 tumor cells expressing eGFP and fLuc were intravenously injected into NSG mice and treated either with anti-CSF1R-CAR T cells or anti-CD19 CAR T cells as a negative control. Treatment with anti-CSF1R-CAR T cells resulted in increased survival of the recipient mice (FIG. 16A) and decreased luminescence signal (FIG. 16B). In addition, anti-CSF1R-CAR T cells were further tested in another PDX AML model with differing disease-associated cytogenetic characteristics (FAB: M1; Cytogenetics: aberrant complex) from the previously used model. Similar to the previous results, treatment with anti-CSF1R-CAR T cells resulted in a decrease in luminescence signal due to strong anti-tumor effect of anti-CSF1R-CAR T cells (FIG. 17A, B), demonstrating the strong anti-tumor efficacy of anti-CSF1R-CAR T cells.

These results further highlight the potential of anti-CSF1R-CAR T cells for treating AML with differing cytogenetic properties and take into consideration the heterogeneity of AML as a disease.

1. A method of treating cancer characterized by the expression of colony stimulating factor 1 receptor (CSF1R), the method comprising administering a CSF1R targeting agent or a lymphocyte recombinantly expressing a chimeric antigen T cell receptor (CAR),
wherein said CAR comprises an extracellular domain that specifically binds CSF1R, a transmembrane domain, and an intracellular T cell activating domain.

2. The method according to claim 1,
wherein said extracellular domain comprises an antigen binding region that is a human or humanized scFv specific for said CSF1R, and a spacer comprising a hinge region which connects said antigen binding region to the transmembrane domain of said CAR, wherein said spacer
(i) does not comprise an antibody Fc region or portion thereof, and/or
(ii) does not have binding activity for one or more Fc receptors.

3. A CAR comprising an extracellular domain that specifically binds CSF1R, a transmembrane domain, and an intracellular T cell activating domain, said extracellular domain comprising
(i) an antigen binding region that is a human or humanized scFv antigen binding region specific for CSF1R; and
(ii) a spacer comprising a human hinge region and a detectable tag allowing the detection and/or purification of said CAR or a cell expressing said CAR which spacer does not comprise an antibody Fc region or portion thereof and/or does not have binding activity for one or more Fc receptors;
wherein said intracellular T cell activating domain comprises the signaling domain of the CD3ζ chain and/or at least one costimulatory domain that is an intracellular domain of an endogenous T cell receptor.

4. The method according to claim 2, wherein said spacer does not have binding activity for one or more Fc receptors, which one or more Fc receptors is an FcγR or FcRn.

5. The method according to claim 2, wherein said hinge region is a CD8 hinge region.

6. The method according to claim 1, wherein said intracellular T cell activating domain comprises the signaling domain of the CD3ζ chain and/or at least one costimulatory domain that is an intracellular domain of an endogenous T cell receptor.

7. The method according to claim 1, wherein said CAR comprises or consists of
(a) the amino acid sequence of SEQ ID NO:23 or SEQ ID NO:24;
(b) an amino acid sequence that is at least 85% identical to SEQ ID NO: 23 or SEQ ID NO:24 and characterized by specifically binding to CSF1R, by having a c-myc tag, and by having T cell activating activity when expressed by a lymphocyte and on binding to CSF1R; or
(c) a fragment of the amino acid sequence of (a) or (b), wherein the fragment is characterized by specifically binding to CSF1R, by having a c-myc tag, and by having T cell activating activity when expressed by a lymphocyte and on binding to CSF1R.

8. The method according to claim 1, wherein said cancer is a hematological cancer.

9. The method according to claim 8, wherein said hematological cancer is acute myeloid leukemia (AML).

10. The method according to claim 1, wherein said specific binding is the specific binding of a lymphocyte recombinantly expressing said CAR to CSF1R.

11. A polynucleotide encoding the CAR according to claim 3.

12. A vector comprising the polynucleotide of claim 11.

13. A host cell comprising the vector according to claim 12, wherein said host cell is a T cell, NK cell, or innate lymphoid cell.

14. A method for producing a lymphocyte recombinantly expressing a chimeric antigen T cell receptor (CAR), the method comprising
(a) introducing into the lymphocyte a polynucleotide encoding the CAR, or an expression vector comprising said polynucleotide;
(b) culturing the lymphocyte recombinantly engineered according to (a) under conditions allowing the expression of the CAR; and
(c) recovering the engineered lymphocyte;
wherein the CAR comprises an extracellular domain that specifically binds CSF1R, a transmembrane domain, and an intracellular T cell activating domain, said extracellular domain comprising
(i) an antigen binding region that is a human or humanized scFv antigen binding region specific for CSF1R; and
(ii) a spacer comprising a human hinge region and a detectable tag allowing the detection and/or purification of said CAR or a cell expressing said CAR which spacer does not comprise an antibody Fc region or portion thereof and/or does not have binding activity for one or more Fc receptors:
wherein said intracellular T cell activating domain comprises the signaling domain of the CD3ζ chain and/or at least one costimulatory domain that is an intracellular domain of an endogenous T cell receptor.

15. A pharmaceutical composition comprising the cell according to claim 13.

16. The method according to claim 6, wherein said costimulatory domain comprises an intracellular domain of at least CD28 and/or CD137(4-1BB).

17. The method according to claim 14, further comprising expanding said lymphocyte in step (b) and/or subsequent to step (c) by exposure to one or more of an anti-CD3 antibody; an anti-CD28 antibody; and one or more cytokines that is at least interleukin-2 (TL-2) or interleukin-15 (IL-15).

18. The CAR according to claim 3, wherein said hinge region is a CD8 hinge region

19. The CAR according to claim 3, wherein said costimulatory domain comprises an intracellular domain of at least CD28 and/or CD137(4-1BB).

20. The CAR according to claim 3, comprising or consisting of:
(a) the amino acid sequence of SEQ ID NO:23 or SEQ ID NO:24;
(b) an amino acid sequence that is at least 85% identical to SEQ ID NO: 23 or SEQ ID NO:24, and characterized by specifically binding to CSF1R, by having a c-myc tag and by having T cell activating activity when expressed by a lymphocyte and on binding to CSF1R; or
(c) a fragment of the amino acid sequence of (a) or (b), wherein the fragment is characterized by specifically binding to CSF1R, by having a c-myc tag and by having T cell activating activity when expressed by a lymphocyte and on binding to CSF1R.


SEQUENCE LISTING (38 sequences)

SEQ ID NO: 1 - protein, 242 aa, synthetic construct
  REGION 1..242: note = artificially synthesized sequence
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSGGGGSGGGGSGGGGSGGGGSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIK

SEQ ID NO: 2 - protein, 253 aa, synthetic construct
  REGION 1..253: note = artificially synthesized sequence
DIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKGGGGSGGGGSGGGGSGGGGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWQQGTLVTVSS

SEQ ID NO: 3 - DNA, 726 bp, synthetic construct
  misc_feature 1..726: note = artificially synthesized sequence
  source 1..726: mol_type = other DNA
caggtgcagctggtgcagagcggcgccgaggtgaagaagcccggcgccagcgtgaaggtgagctgcaaggccagcggctacaccttcaccagctacgacatcagctgggtgaggcaggcccccggccagggcctggagtggatgggcgtgatctggaccgacggcggcaccaactacgcccagaagctgcagggcagggtgaccatgaccaccgacaccagcaccagcaccgcctacatggagctgaggagcctgaggagcgacgacaccgccgtgtactactgcgccagggaccagaggctgtacttcgacgtgtggggccagggcaccaccgtgaccgtgagcagcggaggaggtggaagtggaggtggaggctcaggtggtggggggagcggaggagggggctctgacatccagatgacccagagccccagcagcctgagcgccagcgtgggcgacagggtgaccatcacctgcagggccagcgaggacgtgaacacctacgtgagctggtaccagcagaagcccggcaaggcccccaagctgctgatctacgccgccagcaacaggtacaccggcgtgcccagcaggttcagcggcagcggcagcggcaccgacttcaccctgaccatcagcagcctgcagcccgaggacttcgccacctactactgccagcagagcttcagctaccccaccttcggccagggcaccaagctggagatcaag

SEQ ID NO: 4 - DNA, 759 bp, synthetic construct
  misc_feature 1..759: note = artificially synthesized sequence
  source 1..759: mol_type = other DNA
gacatcgtgatgacccagagccccgacagcctggccgtgagcctgggcgagagggccaccatcaactgcaagagcagccagagcgtgctggacagcagcgacaacaagaactacctggcctggtaccagcagaagcccggccagccccccaagctgctgatctactgggccagcaacagggagagcggcgtgcccgacaggttcagcggcagcggcagcggcaccgacttcaccctgaccatcagcagcctgcaggccgaggacgtggccgtgtactactgccagcagtactacagcgaccccttcaccttcggccccggcaccaaggtggacatcaagggaggaggtggctcaggtggtggaggatctggaggaggtgggagtggtggaggtggttctcaggtgcagctggtgcagagcggcgccgaggtgaagaagcccggcgccagcgtgaaggtgagctgcaaggccagcggctacaccttcaccagctacggcatcagctgggtgaggcaggcccccggccagggcctggagtggatgggctggatcagcgcctacaacggcaacaccaactacgcccagaagctgcagggcagggtgaccatgaccaccgacaccagcaccagcaccgcctacatggagctgaggagcctgaggagcgacgacaccgccgtgtactactgcgccagggagagctggttcggcgaggtgttcttcgactactggcagcagggcaccctggtgaccgtgagcagc

SEQ ID NO: 5 - protein, 45 aa, Mus musculus
TTTKPVLRTPSPVHPTGTSQPQRPEDCRPRGSVKGTGLDFACDIY

SEQ ID NO: 6 - DNA, 135 bp, Mus musculus
  source 1..135: mol_type = unassigned DNA
actactaccaagccagtgctgcgaactccctcacctgtgcaccctaccgggacatctcagccccagagaccagaagattgtcggccccgtggctcagtgaaggggaccggattggacttcgcctgtgatatttac

SEQ ID NO: 7 - protein, 45 aa, Homo sapiens
TTTPAPRPPTPAPTIASQPLSLRPEACRPAAGGAVHTRGLDFACD

SEQ ID NO: 8 - DNA, 135 bp, Homo sapiens
  source 1..135: mol_type = unassigned DNA
accacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgat

SEQ ID NO: 9 - protein, 39 aa, Homo sapiens
IEVMYPPPYLDNEKSNGTIIHVKGKHLCPSPLFPGPSKP

SEQ ID NO: 10 - DNA, 117 bp, Homo sapiens
  source 1..117: mol_type = unassigned DNA
atcgaggtgatgtacccccccccctacctggacaacgagaagagcaacggcaccatcatccacgtgaagggcaagcacctgtgccccagccccctgttccccggccccagcaagccc

SEQ ID NO: 11 - protein, 10 aa, synthetic construct
  REGION 1..10: note = artificially synthesized sequence
EQKLISEEDL

SEQ ID NO: 12 - DNA, 30 bp, synthetic construct
  misc_feature 1..30: note = artificially synthesized sequence
  source 1..30: mol_type = other DNA
gagcaaaagctcatttctgaagaggacttg

SEQ ID NO: 13 - protein, 55 aa, synthetic construct
  REGION 1..55: note = artificially synthesized sequence
EQKLISEEDLTTTPAPRPPTPAPTIASQPLSLRPEACRPAAGGAVHTRGLDFACD

SEQ ID NO: 14 - protein, 49 aa, synthetic construct
  REGION 1..49: note = artificially synthesized sequence
EQKLISEEDLIEVMYPPPYLDNEKSNGTIIHVKGKHLCPSPLFPGPSKP

SEQ ID NO: 15 - protein, 297 aa, synthetic construct
  REGION 1..297: note = artificially synthesized sequence
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSGGGGSGGGGSGGGGSGGGGSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIKEQKLISEEDLTTTPAPRPPTPAPTIASQPLSLRPEACRPAAGGAVHTRGLDFACD

SEQ ID NO: 16 - protein, 308 aa, synthetic construct
  REGION 1..308: note = artificially synthesized sequence
DIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKGGGGSGGGGSGGGGSGGGGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWQQGTLVTVSSEQKLISEEDLTTTPAPRPPTPAPTIASQPLSLRPEACRPAAGGAVHTRGLDFACD

SEQ ID NO: 17 - protein, 291 aa, synthetic construct
  REGION 1..291: note = artificially synthesized sequence
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSGGGGSGGGGSGGGGSGGGGSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIKEQKLISEEDLIEVMYPPPYLDNEKSNGTIIHVKGKHLCPSPLFPGPSKP

SEQ ID NO: 18 - protein, 302 aa, synthetic construct
  REGION 1..302: note = artificially synthesized sequence
DIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKGGGGSGGGGSGGGGSGGGGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWQQGTLVTVSSEQKLISEEDLIEVMYPPPYLDNEKSNGTIIHVKGKHLCPSPLFPGPSKP

SEQ ID NO: 19 - protein, 28 aa, Mus musculus
FWALVVVAGVLFCYGLLVTVALCVIWTN

SEQ ID NO: 20 - DNA, 84 bp, Mus musculus
  source 1..84: mol_type = unassigned DNA
ttttgggcactggtcgtggttgctggagtcctgttttgttatggcttgctagtgacagtggctctttgtgttatctggacaaat

SEQ ID NO: 21 - protein, 27 aa, Homo sapiens
FWVLVVVGGVLACYSLLVTVAFIIFWV

SEQ ID NO: 22 - DNA, 81 bp, Homo sapiens
  source 1..81: mol_type = unassigned DNA
ttttgggtgctggtggtggttggtggagtcctggcttgctatagcttgctagtaacagtggcctttattattttctgggtg

SEQ ID NO: 23 - protein, 478 aa, synthetic construct
  REGION 1..478: note = artificially synthesized sequence
QVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSGGGGSGGGGSGGGGSGGGGSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIKEQKLISEEDLTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 24 - protein, 489 aa, synthetic construct
  REGION 1..489: note = artificially synthesized sequence
DIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKGGGGSGGGGSGGGGSGGGGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWQQGTLVTVSSEQKLISEEDLTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 25 - protein, 501 aa, synthetic construct
  REGION 1..501: note = artificially synthesized sequence
ATMALPVTALLLPLALLLHAARPQVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSGGGGSGGGGSGGGGSGGGGSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIKEQKLISEEDLTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 26 - DNA, 1497 bp, synthetic construct
  misc_feature 1..1497: note = artificially synthesized sequence
  source 1..1497: mol_type = other DNA
atggccttaccagtgaccgccttgctcctgccgctggccttgctgctccacgccgccaggccgcaggtgcagctggtgcagagcggcgccgaggtgaagaagcccggcgccagcgtgaaggtgagctgcaaggccagcggctacaccttcaccagctacgacatcagctgggtgaggcaggcccccggccagggcctggagtggatgggcgtgatctggaccgacggcggcaccaactacgcccagaagctgcagggcagggtgaccatgaccaccgacaccagcaccagcaccgcctacatggagctgaggagcctgaggagcgacgacaccgccgtgtactactgcgccagggaccagaggctgtacttcgacgtgtggggccagggcaccaccgtgaccgtgagcagcggaggaggtggctcaggtggtggaggatctggaggaggtgggagtggtggaggtggttctgacatccagatgacccagagccccagcagcctgagcgccagcgtgggcgacagggtgaccatcacctgcagggccagcgaggacgtgaacacctacgtgagctggtaccagcagaagcccggcaaggcccccaagctgctgatctacgccgccagcaacaggtacaccggcgtgcccagcaggttcagcggcagcggcagcggcaccgacttcaccctgaccatcagcagcctgcagcccgaggacttcgccacctactactgccagcagagcttcagctaccccaccttcggccagggcaccaagctggagatcaaggagcaaaagctcatttctgaagaggacttgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 27 - protein, 510 aa, synthetic construct
  REGION 1..510: note = artificially synthesized sequence
MALPVTALLLPLALLLHAARPDIVMTQSPDSLAVSLGERATINCKSSQSVLDSSDNKNYLAWYQQKPGQPPKLLIYWASNRESGVPDRFSGSGSGTDFTLTISSLQAEDVAVYYCQQYYSDPFTFGPGTKVDIKGGGGSGGGGSGGGGSGGGGSQVQLVQSGAEVKKPGASVKVSCKASGYTFTSYGISWVRQAPGQGLEWMGWISAYNGNTNYAQKLQGRVTMTTDTSTSTAYMELRSLRSDDTAVYYCARESWFGEVFFDYWQQGTLVTVSSEQKLISEEDLTTTPAPRPPTPAPTIASQPLSLRPEACRPAAGGAVHTRGLDFACDFWVLVVVGGVLACYSLLVTVAFIIFWVRSKRSRLLHSDYMNMTPRRPGPTRKHYQPYAPPRDFAAYRSRVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 28 - DNA, 1530 bp, synthetic construct
  misc_feature 1..1530: note = artificially synthesized sequence
  source 1..1530: mol_type = other DNA
atggccttaccagtgaccgccttgctcctgccgctggccttgctgctccacgccgccaggccggacatcgtgatgacccagagccccgacagcctggccgtgagcctgggcgagagggccaccatcaactgcaagagcagccagagcgtgctggacagcagcgacaacaagaactacctggcctggtaccagcagaagcccggccagccccccaagctgctgatctactgggccagcaacagggagagcggcgtgcccgacaggttcagcggcagcggcagcggcaccgacttcaccctgaccatcagcagcctgcaggccgaggacgtggccgtgtactactgccagcagtactacagcgaccccttcaccttcggccccggcaccaaggtggacatcaagggaggaggtggctcaggtggtggaggatctggaggaggtgggagtggtggaggtggttctcaggtgcagctggtgcagagcggcgccgaggtgaagaagcccggcgccagcgtgaaggtgagctgcaaggccagcggctacaccttcaccagctacggcatcagctgggtgaggcaggcccccggccagggcctggagtggatgggctggatcagcgcctacaacggcaacaccaactacgcccagaagctgcagggcagggtgaccatgaccaccgacaccagcaccagcaccgcctacatggagctgaggagcctgaggagcgacgacaccgccgtgtactactgcgccagggagagctggttcggcgaggtgttcttcgactactggcagcagggcaccctggtgaccgtgagcagcgagcaaaagctcatttctgaagaggacttgaccacgacgccagcgccgcgaccaccaacaccggcgcccaccatcgcgtcgcagcccctgtccctgcgcccagaggcgtgccggccagcggcggggggcgcagtgcacacgagggggctggacttcgcctgtgatttttgggtgctggtggtggttggtggagtcctggcttgctatagcttgctagtaacagtggcctttattattttctgggtgaggagtaagaggagcaggctcctgcacagtgactacatgaacatgactccccgccgccccgggcccacccgcaagcattaccagccctatgccccaccacgcgacttcgcagcctatcgctccagagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 29 - protein, 113 aa, Mus musculus
RAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 30 - DNA, 339 bp, Mus musculus
  source 1..339: mol_type = unassigned DNA
agagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgc

SEQ ID NO: 31 - protein, 40 aa, Mus musculus
SRRNRLLQSDYMNMTPRRPGLTRKPYQPYAPARDFAAYRP

SEQ ID NO: 32 - DNA, 120 bp, Mus musculus
  source 1..120: mol_type = unassigned DNA
agtagaaggaacagactccttcaaagtgactacatgaacatgactccccggaggcctgggctcactcgaaagccttaccagccctacgcccctgccagagactttgcagcgtaccgcccc

SEQ ID NO: 33 - protein, 113 aa, Homo sapiens
RVKFSRSADAPAYQQGQNQLYNELNLGRREEYDVLDKRRGRDPEMGGKPQRRKNPQEGLYNELQKDKMAEAYSEIGMKGERRRGKGHDGLYQGLSTATKDTYDALHMQALPPR

SEQ ID NO: 34 - DNA, 339 bp, Homo sapiens
  source 1..339: mol_type = unassigned DNA
agagtgaagttcagcaggagcgcagacgcccccgcgtaccagcagggccagaaccagctctataacgagctcaatctaggacgaagagaggagtacgatgttttggacaagagacgtggccgggaccctgagatggggggaaagccgcagagaaggaagaaccctcaggaaggcctgtacaatgaactgcagaaagataagatggcggaggcctacagtgagattgggatgaaaggcgagcgccggaggggcaaggggcacgatggcctttaccagggtctcagtacagccaccaaggacacctacgacgcccttcacatgcaggccctgccccctcgc

SEQ ID NO: 35 - protein, 41 aa, Homo sapiens
RSKRSRLLHSDYMNMTPRRPGPTRKHYQPYAPPRDFAAYRS

SEQ ID NO: 36 - DNA, 123 bp, Homo sapiens
  source 1..123: mol_type = unassigned DNA
aggagtaagaggagcaggctcctgcacagtgactacatgaacatgactccccgccgccccgggcccacccgcaagcattaccagccctatgccccaccacgcgacttcgcagcctatcgctcc

SEQ ID NO: 37 - protein, 499 aa, synthetic construct
  REGION 1..499: note = artificially synthesized sequence
MALPVTALLLPLALLLHAARPQVQLVQSGAEVKKPGASVKVSCKASGYTFTSYDISWVRQAPGQGLEWMGVIWTDGGTNYAQKLQGRVTMTTDTSTSTAYMELRSLRSDDTAVYYCARDQRLYFDVWGQGTTVTVSSGGGGSGGGGSGGGGSGGGGSDIQMTQSPSSLSASVGDRVTITCRASEDVNTYVSWYQQKPGKAPKLLIYAASNRYTGVPSRFSGSGSGTDFTLTISSLQPEDFATYYCQQSFSYPTFGQGTKLEIKEQKLISEEDLTTTKPVLRTPSPVHPTGTSQPQRPEDCRPRGSVKGTGLDFACDIYFWALVVVAGVLFCYGLLVTVALCVIWTNSRRNRLLQSDYMNMTPRRPGLTRKPYQPYAPARDFAAYRPRAKFSRSAETAANLQDPNQLYNELNLGRREEYDVLEKKRARDPEMGGKQQRRRNPQEGVYNALQKDKMAEAYSEIGTKGERRRGKGHDGLYQGLSTATKDTYDALHMQTLAPR

SEQ ID NO: 38 - DNA, 1497 bp, synthetic construct
  misc_feature 1..1497: note = artificially synthesized sequence
  source 1..1497: mol_type = other DNA
atggccttaccagtgaccgccttgctcctgccgctggccttgctgctccacgccgccaggccgcaggtgcagctggtgcagagcggcgccgaggtgaagaagcccggcgccagcgtgaaggtgagctgcaaggccagcggctacaccttcaccagctacgacatcagctgggtgaggcaggcccccggccagggcctggagtggatgggcgtgatctggaccgacggcggcaccaactacgcccagaagctgcagggcagggtgaccatgaccaccgacaccagcaccagcaccgcctacatggagctgaggagcctgaggagcgacgacaccgccgtgtactactgcgccagggaccagaggctgtacttcgacgtgtggggccagggcaccaccgtgaccgtgagcagcggaggaggtggaagtggaggtggaggctcaggtggtggggggagcggaggagggggctctgacatccagatgacccagagccccagcagcctgagcgccagcgtgggcgacagggtgaccatcacctgcagggccagcgaggacgtgaacacctacgtgagctggtaccagcagaagcccggcaaggcccccaagctgctgatctacgccgccagcaacaggtacaccggcgtgcccagcaggttcagcggcagcggcagcggcaccgacttcaccctgaccatcagcagcctgcagcccgaggacttcgccacctactactgccagcagagcttcagctaccccaccttcggccagggcaccaagctggagatcaaggagcaaaagctcatttctgaagaggacttgactactaccaagccagtgctgcgaactccctcacctgtgcaccctaccgggacatctcagccccagagaccagaagattgtcggccccgtggctcagtgaaggggaccggattggacttcgcctgtgatatttacttttgggcactggtcgtggttgctggagtcctgttttgttatggcttgctagtgacagtggctctttgtgttatctggacaaatagtagaaggaacagactccttcaaagtgactacatgaacatgactccccggaggcctgggctcactcgaaagccttaccagccctacgcccctgccagagactttgcagcgtaccgccccagagcaaaattcagcaggagtgcagagactgctgccaacctgcaggaccccaaccagctctacaatgagctcaatctagggcgaagagaggaatatgacgtcttggagaagaagcgggctcgggatccagagatgggaggcaaacagcagaggaggaggaacccccaggaaggcgtatacaatgcactgcagaaagacaagatggcagaagcctacagtgagatcggcacaaaaggcgagaggcggagaggcaaggggcacgatggcctttaccagggtctcagcactgccaccaaggacacctatgatgccctgcatatgcagaccctggcccctcgc